SEQUENCE LISTING (11 sequences)

SEQ ID NO: 1 - DNA, 20 bp, Artificial Sequence
  CpG oligonucleotide
tccatgacgttcctgacgtt

SEQ ID NO: 2 - DNA, 18 bp, Artificial Sequence
  CpG oligonucleotide
tctcccagcgtgcgccat

SEQ ID NO: 3 - DNA, 30 bp, Artificial Sequence
  CpG oligonucleotide
accgatgacgtcgccggtgacggcaccacg

SEQ ID NO: 4 - DNA, 24 bp, Artificial Sequence
  CpG oligonucleotide
tcgtcgttttgtcgttttgtcgtt

SEQ ID NO: 5 - DNA, 20 bp, Artificial Sequence
  CpG oligonucleotide
tccatgacgttcctgatgct

SEQ ID NO: 6 - DNA, 22 bp, Artificial Sequence
  CpG oligonucleotide
tcgacgttttcggcgcgcgccg

SEQ ID NO: 7 - DNA, 3411 bp, Artificial Sequence
  clade B F4 fusion
atggttatcgtgcagaacatccaggggcaaatggtacatcaggccatatcacctagaactttaaatgcatgggtaaaagtagtagaagagaaggctttcagcccagaagtaatacccatgttttcagcattatcagaaggagccaccccacaagatttaaacaccatgctaaacacagtggggggacatcaagcagccatgcaaatgttaaaagagaccatcaatgaggaagctgcagaatgggatagagtacatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccttttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttgcatatgggccccattagccctattgagactgtgtcagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaaatatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattccttaaaatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtgctagctatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgcaggcctatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattactaa

SEQ ID NO: 8 - protein, 1136 aa, Artificial Sequence
  clade B F4 fusion
MVIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLHMGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLAMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

SEQ ID NO: 9 - DNA, 3411 bp, Artificial Sequence
  clade B F4co
atggtcattgttcagaacatacagggccaaatggtccaccaggcaattagtccgcgaactcttaatgcatgggtgaaggtcgtggaggaaaaggcattctccccggaggtcattccgatgttttctgcgctatctgagggcgcaacgccgcaagaccttaataccatgcttaacacggtaggcgggcaccaagccgctatgcaaatgctaaaagagactataaacgaagaggccgccgaatgggatcgagtgcacccggtgcacgccggcccaattgcaccaggccagatgcgcgagccgcgcgggtctgatattgcaggaactacgtctacccttcaggagcagattgggtggatgactaacaatccaccaatcccggtcggagagatctataagaggtggatcatactgggactaaacaagatagtccgcatgtattctccgacttctatactggatatacgccaaggcccaaaggagccgttcagggactatgtcgaccgattctataagacccttcgcgcagagcaggcatcccaggaggtcaaaaattggatgacagaaactcttttggtgcagaatgcgaatccggattgtaaaacaattttaaaggctctaggaccggccgcaacgctagaagagatgatgacggcttgtcagggagtcggtggaccggggcataaagcccgcgtcttacacatgggcccgatatctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaaaaaggattcaacaaaatggcgaaagcttgtagattttagggaactaaacaagcgaacccaagacttttgggaagtccaactagggatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctgtatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacggggtatactacgacccctccaaggaccttatagccgagatccagaagcaggggcagggccaatggacgtaccagatatatcaagaaccgtttaagaatctgaagactgggaagtacgcgcgcatgcgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccacttgttaagctttggtaccagcttgaaaaggagccgatagtaggggcagagaccttctatgtcgatggcgccgcgaatcgcgaaacgaagctaggcaaggcgggatacgtgactaataggggccgccaaaaggtcgtaacccttacggataccaccaatcagaagactgaactacaagcgatttaccttgcacttcaggatagtggcctagaggtcaacatagtcacggactctcaatatgcgcttggcattattcaagcgcagccagatcaaagcgaaagcgagcttgtaaaccaaataatagaacagcttataaagaaagagaaggtatatctggcctgggtccccgctcacaagggaattggcggcaatgagcaagtggacaagctagtcagcgctgggattcgcaaggttcttgcgatggggggtaagtggtctaagtctagcgtagtcggctggccgacagtccgcgagcgcatgcgacgcgccgaaccagccgcagatggcgtgggggcagcgtctagggatctggagaagcacggggctataacttccagtaacacggcggcgacgaacgccgcatgcgcatggttagaagcccaagaagaggaagaagtagggtttccggtaactccccaggtgccgttaaggccgatgacctataaggcagcggtggatctttctcacttccttaaggagaaaggggggctggagggcttaattcacagccagaggcgacaggatattcttgatctgtggatttaccatacccaggggtactttccggactggcagaattacaccccggggccaggcgtgcgctatcccctgactttcgggtggtgctacaaactagtcccagtggaacccgacaaggtcgaagaggctaataagggcgagaacacttctcttcttcacccggtaagcctgcacgggatggatgacccagaacgagaggttctagaatggaggttcgactctcgacttgcgttccatcacgtagcacgcgagctgcatccagaatatttcaagaactgccgcccaatgggcgccagggccagtgtacttagtggcggagaactagatcgatgggaaaagatacgcctacgcccggggggcaagaagaagtacaagcttaagcacattgtgtgggcctctcgcgaacttgagcgattcgcagtgaatccaggcctgcttgagacgagtgaaggctgtaggcaaattctggggcagctacagccgagcctacagactggcagcgaggagcttcgtagtctttataataccgtcgcgactctctactgcgttcatcaacgaattgaaataaaggatactaaagaggcccttgataaaattgaggaggaacagaataagtcgaaaaagaaggcccagcaggccgccgccgacaccgggcacagcaaccaggtgtcccaaaactactaa

SEQ ID NO: 10 - protein, 1140 aa, Artificial Sequence
  10 clade C F4
MVIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIKQGPKEPFRDYVDRFFKTLRAEQATQEVKNWMTDTLLVQNANPDCKTILRALGPGATLEEMMTACQGVGGPGHKARVLHMGPISPIETVPVKLKPGMDGPKVKQWPLTEEKIKALTAICEEMEKEGKITKIGPENPYNTPVFAIKKKDSTKWRKLVDFRELNKRTQDFWEVQLGIPHPAGLKKKKSVTVLDVGDAYFSVPLDEGFRKYTAFTIPSINNETPGIRYQYNVLPQGWKGSPAIFQSSMTKILEPFRAQNPEIVIYQYMDDLYVGSDLEIGQHRAKIEELREHLLKWGFTTPDKKHQKEPPFLKMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKEPIAGAETFYVDGAANRETKIGKAGYVTDRGRQKVVSLTETTNQKTELQAIQLALQDSGSEVNIVTDSQYALGIIQAQPDKSESELVNQIIEQLIKKERVYLSWVPAHKGIGGNEQVDKLVSSGIRKVLAMGGKWSKSSIVGWPAIRERMRRTEPAAEGVGAASQDLDKHGALTSSNTATNNADCAWLEAQEEEEEVGFPVRPQVPLRPMTYKAAFDLSFFLKEKGGLEGLIYSKKRQDILDLWVYHTQGFFPDWQNYTPGPGVRYPLTFGWCYKLVPVDPREVEEANEGENNCLLHPMSQHGMEDEDREVLKWKFDSHLARRHMARELHPEYYKDCRPMGARASILRGGKLDKWEKIRLRPGGKKHYMLKHLVWASRELERFALNPGLLETSEGCKQIIKQLQPALQTGTEELRSLYNTVATLYCVHAKIEVRDTKEALDKIEEEQNKSQQKTQQAKAADGKVSQNYHHHHHH

SEQ ID NO: 11 - DNA, 3423 bp, Artificial Sequence
  11 clade C F4
atggttattgttcagaatctgcagggtcagatggttcatcaggcaatttctccgcgtaccctgaatgcatgggtgaaagtgattgaagaaaaagccttttctccggaagttattccgatgtttaccgcactgagcgaaggtgcaacaccgcaggatctgaataccatgctgaataccgttggtggtcatcaggcagcaatgcagatgctgaaagataccattaatgaagaggcagcagaatgggatcgtctgcatccggttcatgcaggtccgattgcaccgggtcagatgcgtgaaccgcgtggtagcgatattgcaggtacaaccagcaccctgcaagagcagattgcatggatgaccagcaatcctccgattccggttggtgatatttataaacgctggattattctgggcctgaataaaattgtgcgtatgtattctccggttagcattctggatattaaacagggtccgaaagaaccgtttcgtgattatgtggatcgcttttttaaaaccctgcgtgcagaacaggcaacccaagaggttaaaaattggatgaccgataccctgctggttcagaatgcaaatccggattgcaaaaccattctgcgtgcactgggtccgggtgcaacactggaagaaatgatgaccgcatgtcagggtgttggtggtccgggtcataaagcacgtgttctgcacatgggtccgattagcccgattgaaaccgttccggtgaaactgaaaccgggtatggatggtccgaaagttaaacagtggcctctgaccgaagaaaaaatcaaagccctgaccgcaatttgtgaagaaatggaaaaagaaggcaaaattaccaaaattggtccggaaaatccgtataacacaccggtgtttgccattaaaaaaaaagatagcaccaaatggcgtaaactggtggattttcgcgaactgaataaacgtacccaggatttttgggaagttcagctgggtattccgcatccggcaggtctgaaaaaaaaaaaatccgtgaccgttctggatgttggtgatgcctatttttctgttccgctggatgaaggttttcgtaaatataccgcctttaccattccgagcattaataatgaaacaccgggtattcgctatcagtataatgttctgccgcagggttggaaaggttctccggcaatttttcagagcagcatgaccaaaattctggaaccgtttcgcgcacagaatccggaaattgtgatttatcagtatatggatgatctgtatgttggtagcgatctggaaattggtcagcatcgtgccaaaattgaagaactgcgtgaacatctgctgaaatggggttttaccacaccggataaaaaacatcagaaagaaccgccgtttctgaaaatgggttatgaactgcatccggataaatggaccgttcagccgattcagctgccggaaaaagatagctggaccgtgaatgatattcagaaactggtgggcaaactgaattgggcaagccagatttatccgggtattaaagttcgtcagctgtgtaaactgctgcgtggtgcaaaagcactgaccgatattgttccgctgacagaagaagcagaactggaactggccgaaaatcgtgaaattctgaaagaaccggtgcatggtgtttattatgatccgagcaaagatctgattgccgaaattcagaaacagggtcatgatcagtggacctatcagatttatcaggaaccgtttaaaaatctgaaaaccggcaaatatgcaaaaatgcgtaccgcacataccaatgatgttaaacagctgaccgaagccgttcagaaaattgccatggaaagcattgtgatttggggtaaaacaccgaaatttcgtctgccgattcagaaagaaacctgggaaacatggtggaccgattattggcaggcaacctggattccggaatgggaatttgttaatacaccgccgctggttaaactgtggtatcagctggaaaaagaaccgattgcaggtgcagaaaccttttatgttgatggtgcagcaaatcgcgaaaccaaaattggcaaagccggttatgttaccgatcgtggtcgtcagaaagttgttagcctgaccgaaaccaccaatcagaaaaccgaactgcaggcaattcagctggccctgcaggatagcggtagcgaagttaatattgtgaccgatagccagtatgcactgggtattattcaggcacagccggataaaagcgaaagcgaactggtgaatcagattattgaacagctgattaaaaaagaacgcgtgtatctgagctgggttccggcacataaaggtattggtggcaatgaacaggttgataaactggttagcagcggtattcgtaaagttctggccatgggtggtaaatggtctaaaagcagcattgttggttggccggcaattcgtgaacgtatgcgtcgtaccgaaccggcagcagaaggtgttggcgcagcaagccaggatctggataaacatggtgcactgaccagcagcaataccgcaaccaataatgcagattgtgcatggctggaagcacaggaagaagaagaagaagttggttttccggttcgtccgcaggttccgctgcgtccgatgacctataaagcagcatttgatctgagcttttttctgaaagaaaaaggtggtctggaaggtctgatttatagcaaaaaacgccaggatattctggatctgtgggtttatcatacccagggtttttttccggattggcagaattacacaccgggtccgggtgtgcgttatccgctgacctttggttggtgttataaactggttccggttgatccgcgtgaagttgaagaagcaaacgaaggcgaaaataattgtctgctgcatccgatgagccagcatggtatggaagatgaagatcgcgaagtgctgaaatggaaatttgatagccatctggctcgtcgtcacatggcacgcgaactgcatccggaatattataaagattgccgtccgatgggtgcacgtgcaagcattctgcgtggtggtaaactggataaatgggaaaaaattcgtctgcgtccgggtggtaaaaaacattatatgctgaaacatctggtttgggcaagccgtgaactggaacgttttgcactgaatccgggtctgctggaaaccagcgaaggttgcaaacaaattattaaacagctgcagccggcactgcagaccggcaccgaagaactgcgcagcctgtataataccgttgcaaccctgtattgtgtgcatgcgaaaattgaagtgcgcgataccaaagaagcactggataaaattgaagaagaacagaataaaagccagcagaaaacccagcaggcaaaagcagcagatggtaaagtgagccagaattatcaccaccaccaccaccactaa

(19) United States
(12) Patent Application Publication
(10) Pub. No.: US 2014/0234399 A1
(43) Pub. Date: Aug. 21, 2014

(54) HIV VACCINE

(71) Applicant: GlaxoSmithKline Biologicals, s.a., Rixensart (BE)

(72) Inventors: Patricia BOURGUIGNON, Rixensart (BE); Marguerite Christine Koutsoukos, Rixensart (BE); Clarisse Lorin, Rixensart (BE); Lisa McNally, Brentford (GB); Gerald Hermann Voss, Rixensart (BE)

(73) Assignee: GlaxoSmithKline Biologicals, s.a., Rixensart (BE)

(21) Appl. No.: 14/132,267

(22) Filed: Dec. 18, 2013

Related U.S. Application Data
(63) Continuation of application No. 13/637,476.
(60) Provisional application No. 61/318,130, filed on Mar. 26, 2010.

(51) Int. Cl. A61K 39/21

(52) U.S. Cl. CPC A61K 39/21
USPC 424/450; 424/188.1

(57) ABSTRACT

The present invention relates to immunogenic compositions comprising HIV-1 antigens and uses thereof in the prevention and/or treatment of HIV-1. In particular, the invention relates to the use of HIV-1 antigens from one clade in the prevention and/or treatment of disease associated with HIV-1 infection from a heterologous HIV-1 clade.

### RELATED APPLICATIONS

This application is a continuation of U.S. application Ser. No. 13/637,476, filed 26 Sep. 2012, which is the US National Stage of International Application No. PCT/EP2011/054654, filed 25 Mar. 2011, which claims benefit of the filing date of U.S. Provisional Application No. 61/318,130, filed 26 Mar. 2010, which is incorporated herein by reference in its entirety.

### FIELD OF THE INVENTION

The present invention relates to immunogenic compositions comprising HIV-1 antigens and uses thereof in the prevention and/or treatment of HIV-1. In particular, the invention relates to the use of HIV-1 antigens from one clade in the prevention and/or treatment of disease associated with HIV-1 infection from a heterologous HIV-1 clade.

### BACKGROUND TO THE INVENTION

Human immunodeficiency virus type 1 (HIV-1) is the primary cause of the acquired immune deficiency syndrome (AIDS) which is regarded as one of the world's major health problems. With more than 32 million people infected worldwide, the development of a safe and effective vaccine against HIV-1 is a global health priority.

HIV-1 is an RNA virus of the family Retroviridiae. The HIV-1 genome encodes at least nine proteins which are divided into three classes: the major structural proteins Gag, Pol and Env, the regulatory proteins Tat and Rev, and the accessory proteins Vpu, Vpr, Vif and Nef.

HIV-1 can be divided into several different clades, for example A, B, C, D, E, F, G, H, J and K, which vary in prevalence throughout the world. For example, HIV-1 clade B is mostly found throughout North America and Europe, while HIV-1 clade C is largely responsible for the HIV-1 epidemic in South Africa, India and China. Recombinant forms of HIV-1 clades, known as circulating recombinant forms (CRFs) are also known to circulate. These recombinant forms are created when different clades combine within the cell of an infected person to create a new hybrid virus. Most hybrid forms are short-lived, however, those that continue to infect more than one person are known as CRFs. Examples include A/E, which is thought to have resulted from hybridization between subtype A and some other “parent” subtype E. A pure form of subtype E has yet to be found, however.

A virus isolated in Cyprus was originally placed in a new subtype I, before being reclassified as a recombinant form A/G/I. It is now thought that this virus represents an even more complex CRF comprised of subtypes A, G, H, K and unclassified regions.

Each clade comprises different strains of HIV-1 which have been grouped together on the basis of their genetic similarity. The genetic variation between HIV-1 strains from different clades is accordingly greater than the variation between different HIV-1 strains from the same clade.

The genetic diversity of HIV-1 renders extremely difficult the development of a vaccine that is safe and efficacious around the world, against strains from multiple HIV-1 clades. The need for a vaccine that addresses these needs still exists.

In the past two decades, efforts have been made to develop a prophylactic vaccine. To date, only three candidate HIV-1 vaccines have been tested in Phase IIb or III trials and all failed to prevent HIV-1 infection [Flynn N M, Forthal D N, Harro C D, Judson F N, Mayer K H, et al. (2005) Placebo-controlled phase 3 trial of a recombinant glycoprotein 120 vaccine to prevent HIV-1 infection. J Infect Dis 191: 654-665, Pitisuttithum P, Gilbert P, Gurwith M, Heyward W, Martin M, et al. (2006) Randomized, Double-Blind, Placebo-Controlled Efficacy Trial of a Bivalent Recombinant Glycoprotein 120 HIV-1 Vaccine among Injection Drug Users in Bangkok, Thailand. J Infect Dis 194: 1661-1671, Buchbinder S P, Mehrotra D V, Duerr A, Fitzgerald D W, Mogg R, et al. (2008) Efficacy assessment of a cell-mediated immunity HIV-1 vaccine (the Step Study): a double-blind, randomised, placebo-controlled, test-of-concept trial. Lancet 372: 1881-1893]. The development of a prophylactic vaccine which prevents infection leading to sterilizing immunity is a priority; however, a therapeutic or disease-modifying vaccine based on the induction of strong T-cell mediated immune responses is also desirable.

The role of CD8+ T-cell responses in controlling persistent virus infections has been well established [Barouch D H, Letvin N L (2001) CD8+ cytotoxic T lymphocyte responses to lentiviruses and herpesviruses. Curr Opin Immunol 13: 479-482]. In respect of HIV-1 infection, the appearance of virus-specific CD8+ T-cells is closely associated with the drop in viremia that occurs during primary HIV-1 infection [Koup R A, Safrit J T, Cao Y, Andrews C A, McLeod G, et al. (1994) Temporal association of cellular immune responses with the initial control of viremia in primary human immunodeficiency virus type 1 syndrome. J Virol 68: 4650-4655] and depletion of CD8+ T-cells causes a dramatic increase in viremia in simian immunodeficiency virus (SIV) [Schmitz J E, Kuroda M J, Santra S, Sasseville V G, Simon M A, et al. (1999) Control of viremia in simian immunodeficiency virus infection by CD8+ lymphocytes. Science 283: 857-860, Jin X, Bauer D E, Tuttleton S E, Lewin S, Gettie A, et al. (1999) Dramatic rise in plasma viremia after CD8(+) T cell depletion in simian immunodeficiency virus-infected macaques. J Exp Med 189: 991-998]. It has also been found that polyfunctional CD8+ T-cells are preferentially maintained in non-progressors who control HIV-1 infection without highly active anti-retroviral therapy (HAART) [Betts M R, Nason M C, West S M, De Rosa S C, Migueles S A, et al. (2006) HIV nonprogressors preferentially maintain highly functional HIV-specific CD8+ T cells. Blood 107: 4781-4789].

Virus-specific CD4+ T-cells are known to play a central role in the immune control of many viral infections, including HIV-1 [Day C L, Walker B D (2003) Progress in defining CD4 helper cell responses in chronic viral infections. J Exp Med 198: 1773-1777, Klenerman P, Hill A (2005) T cells and viral persistence: lessons from diverse infections. Nat Immunol 6: 873-879]. More specifically, CD4+ T-cells are required for the induction and maintenance of functional CD8+ T-cells [Bourgeois C, Veiga-Fernandes H, Joret A M, Rocha B, Tanchot C (2002) CD8 lethargy in the absence of CD4 help. Eur J Immunol 32: 2199-2207, Janssen E M, Lemmens E E, Wolfe T, Christen U, von Herrath M G, et al. (2003) CD4+ T cells are required for secondary expansion and memory in CD8+ T lymphocytes. Nature 421: 852-856, Shedlock D J, Shen H (2003) Requirement for CD4 T cell help in generating functional CD8 T cell memory. Science 300: 337-339, Sun J C, Bevan M J (2003) Defective CD8 T cell memory following acute infection without CD4 T cell help. Science 300: 339-342, Sun J C, Williams M A, Bevan M J (2004) CD4+ T cells are required for the maintenance, not programming, of memory CD8+ T cells after acute infection. Nat Immunol 5: 927-933, Yang T C, Millar J, Groves T, Zhou W, Grinshtein N, et al. (2007) On the role of CD4+ T cells in the CD8+ T-cell response elicited by recombinant adenovirus vaccines. Mol Ther 15: 997-1006]. The presence of polyfunctional and proliferation-competent HIV-1-specific CD4+ T-cells in HIV-1-infected patients is associated with long-term non-progression [Boaz M J, Waters A, Murad S, Easterbrook P J, Vyakarnam A (2002) Presence of HIV-1 Gag-specific IFN-gamma+IL-2+ and CD28+IL-2+ CD4 T cell responses is associated with nonprogression in HIV-1 infection. J Immunol 169: 6376-6385, Harari A, Petitpierre S, Vallelian F, Pantaleo G (2004) Skewed representation of functionally distinct populations of virus-specific CD4 T cells in HIV-1-infected subjects with progressive disease: changes after antiretroviral therapy. Blood 103: 966-972, Kannanganat S, Kapogiannis B G, Ibegbu C, Chemareddi L, Goepfert P, et al. (2007) Human immunodeficiency virus type 1 controllers but not noncontrollers maintain CD4 T cells coexpressing three cytokines. J Virol 81: 12071-12076, Potter S J, Lacabaratz C, Lambotte O, Perez-Patrigeon S, Vingert B, et al. (2007) Preserved central memory and activated effector memory CD4+ T-cell subsets in human immunodeficiency virus controllers: an ANRS EP36 study. J Virol 81: 13904-13915]. Further, the loss of HIV-1-specific CD8+ T-cell proliferation after acute HIV-1 infection can be restored by vaccine-induced HIV-1-specific CD4+ T-cells that produce IL-2 in vitro and in vivo [Lichterfeld M, Kaufmann D E, Yu X G, Mui S K, Addo M M, et al. (2004) Loss of HIV-1-specific CD8+ T cell proliferation after acute HIV-1 infection and restoration by vaccine-induced HIV-1-specific CD4+ T cells. J Exp Med 200: 701-712]. These findings suggest that an effective AIDS vaccine must also induce a strong CD4+ T-cell response.

Given the immense genetic diversity of HIV-1, an effective vaccine would advantageously induce CD4+ T-cell responses covering a broad spectrum of circulating HIV-1 strains. The need for such a vaccine still exists.

The present invention seeks to address these needs.

### SUMMARY OF THE INVENTION

The present invention provides compositions for the induction of an immune response for the prophylaxis and/or treatment of HIV. In particular, the present invention provides compositions containing HIV antigens for the induction of immune responses against more than one clade of HIV and/or clades heterologous to the HIV antigens used in the composition.

### BRIEF DESCRIPTION OF THE DRAWINGS

FIG. 1: Nucleotide sequence for F4

FIG. 2: Amino acid sequence for F4

FIG. 3: Nucleotide sequence for F4co

FIG. 4: p24 amino acid sequence alignment

FIG. 5: RT amino acid sequence alignment

FIG. 6: Nef amino acid sequence alignment

FIG. 7: p17 amino acid sequence alignment

FIG. 8A-C: Analysis of (A) cross-clade sequence identity and (B)-(C) epitope conservation

FIG. 9A-C: (A) p24-RT-Nef-p17 solubility assay, (B) Coomassie staining and (C) western blot of F4

FIG. 10A-B: (A) Coomassie stained gel and (B) western blot for F4co

FIG. 11A-B: F4co purification follow-up by (A) SDS-PAGE and (B) Western blot analysis

FIGS. 12A-B: (A) CB-stained SDS-gel and (B) the anti-F4 western blot

FIGS. 13A-D: Western blot against the individual antigens in F4

FIG. 14: SEC analysis of the three purification lots

FIG. 15: anti-E. coli western blot

FIGS. 16A-B: (A) SDS-gel and (B) Anti-F4 western blot for all fractions collected during the production of lot 3

FIG. 17: reactogenicity of F4/AS01_{B} and F4/WFI groups

FIG. 18: CD4+ T cells expressing IL-2 and at least another marker p

FIG. 19: CD4+ T-cell response to the F4/AS01_{B}-adjuvanted HIV-1 vaccine candidate: responder rates

FIG. 20: CD4+ T-cell response to the F4/AS01_{B}-adjuvanted HIV-1 vaccine candidate: percentage of responders per antigen

FIGS. 21A-B: Percentage of CD4+ T-cells expressing IL-2 and at least one other marker in response to the F4 fusion protein by dose (A) and over time (B)

FIG. 22: Cytokine co-expression profile of F4-specific CD4+CD40L+ T-cells in the 10 μg F4/AS01_{B} group at 2 weeks post-dose II

FIG. 23: Cytokine co-expression profile of F4-specific CD4+CD40L+ T-cells: pie charts for all time points in the 10 μg F4/AS01_{B} group

FIG. 24A-D: Cytokine co-expression profile of antigen-specific CD4+ T cells by antigen

FIG. 25 A-D: Cytokine co-expression profile of antigen-specific CD4+ T cells by antigen

FIG. 26: Cross-clade reactivity of CD4+ T-cell responses

FIG. 27: Cross-reactivity of CD4+ T-cell responses

FIG. 28A-B: Humoral immune response against the F4 fusion protein by dose (A) and over time (B)

FIG. 29A-D: Antibody response to Nef, RT, p17, p24 antigens by antigen

FIG. 30: Clade A specific CD4+ T cell responses

FIG. 31: Clade B specific CD4+ T cell responses

FIG. 32: Clade C specific CD4+ T cell responses

FIG. 33: Clade A specific CD8+ T cell responses

FIG. 34: Clade B specific CD8+ T cell responses

FIG. 35: Clade C specific CD8+ T cell responses

FIG. 36: Cross-clade T cell response summary

FIG. 37: Study design and subject disposition part 1

FIG. 38: Study design and subject disposition part 2

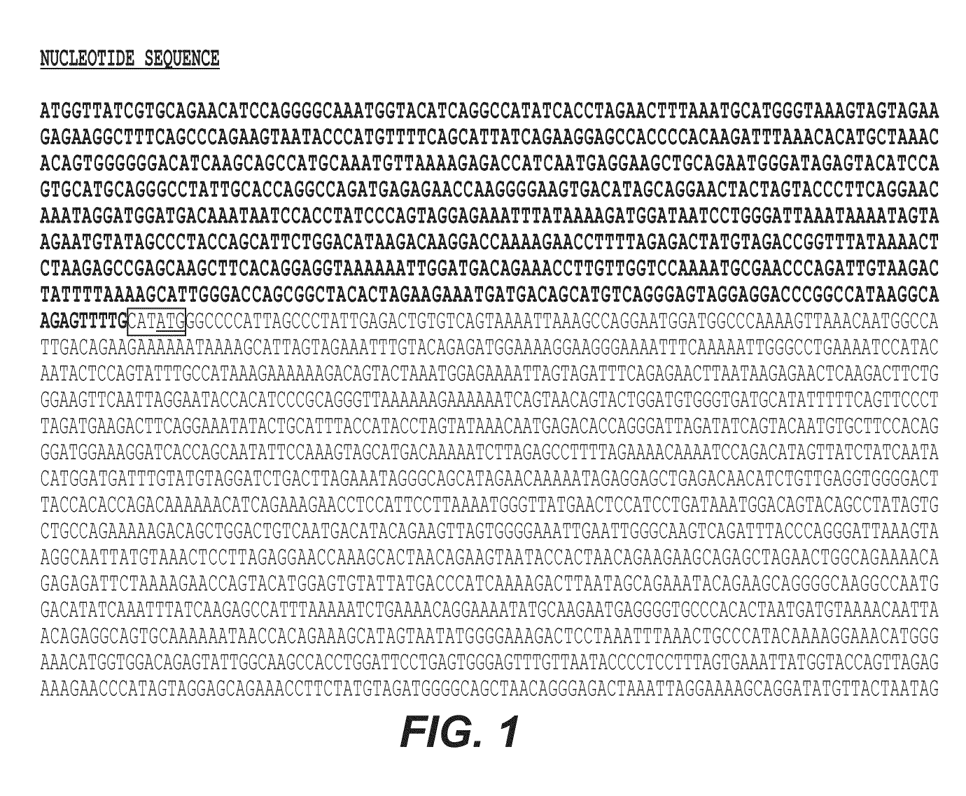

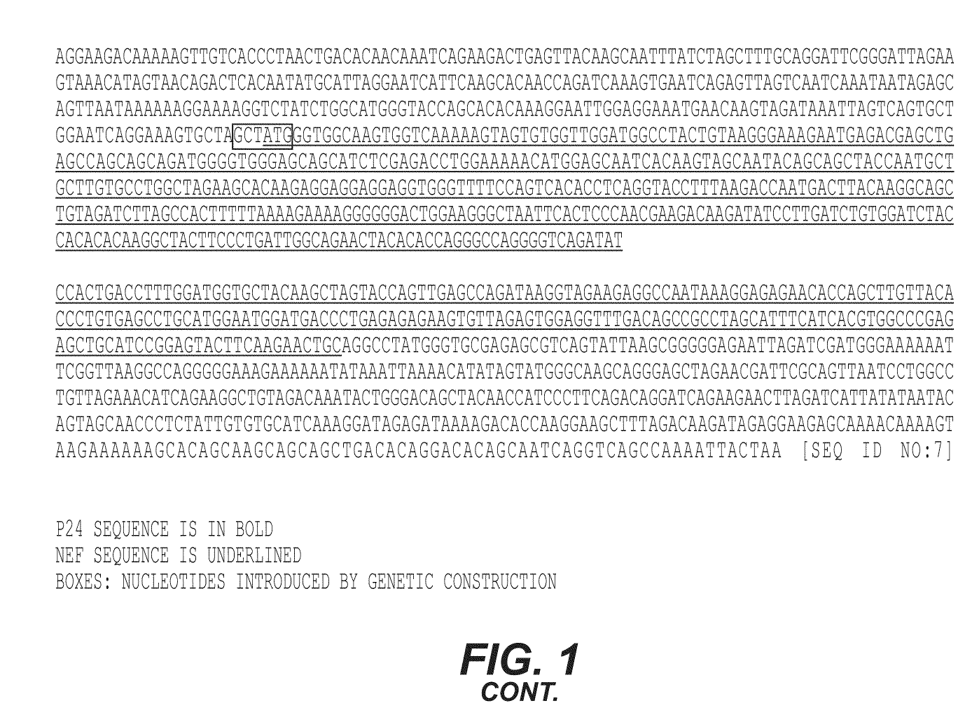

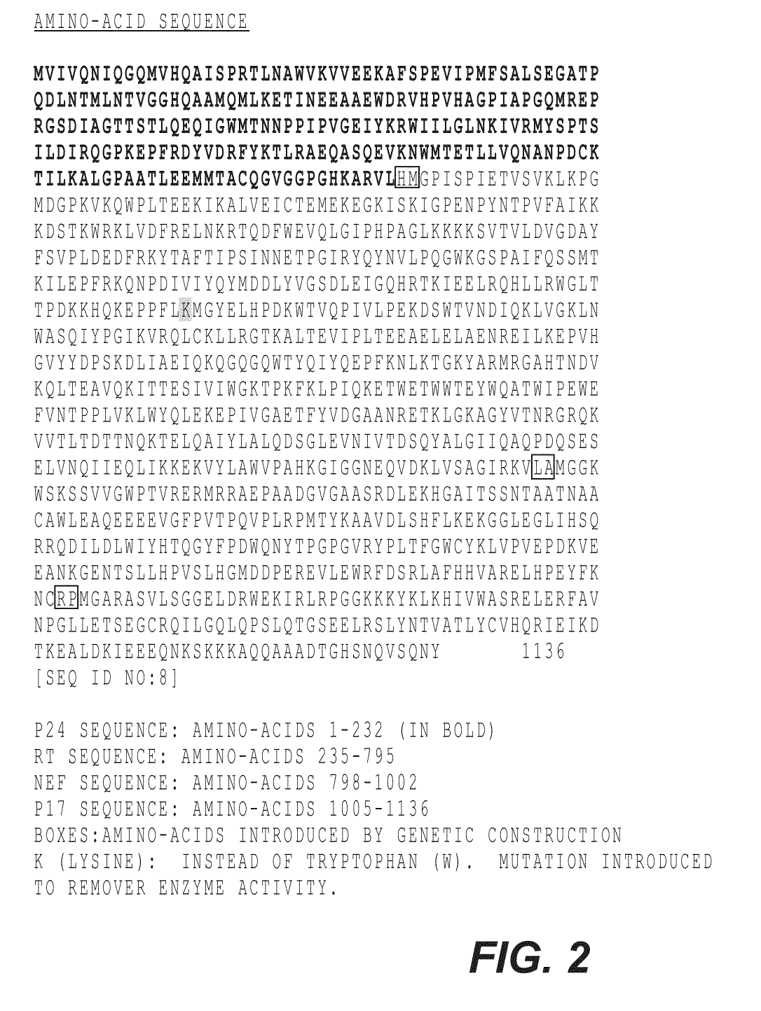

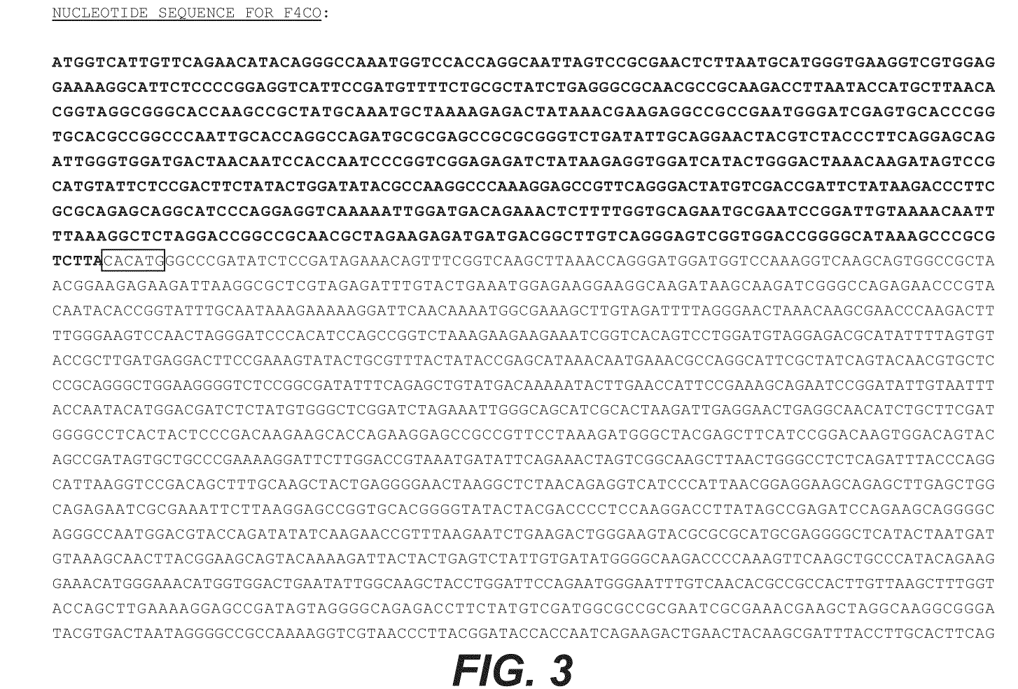

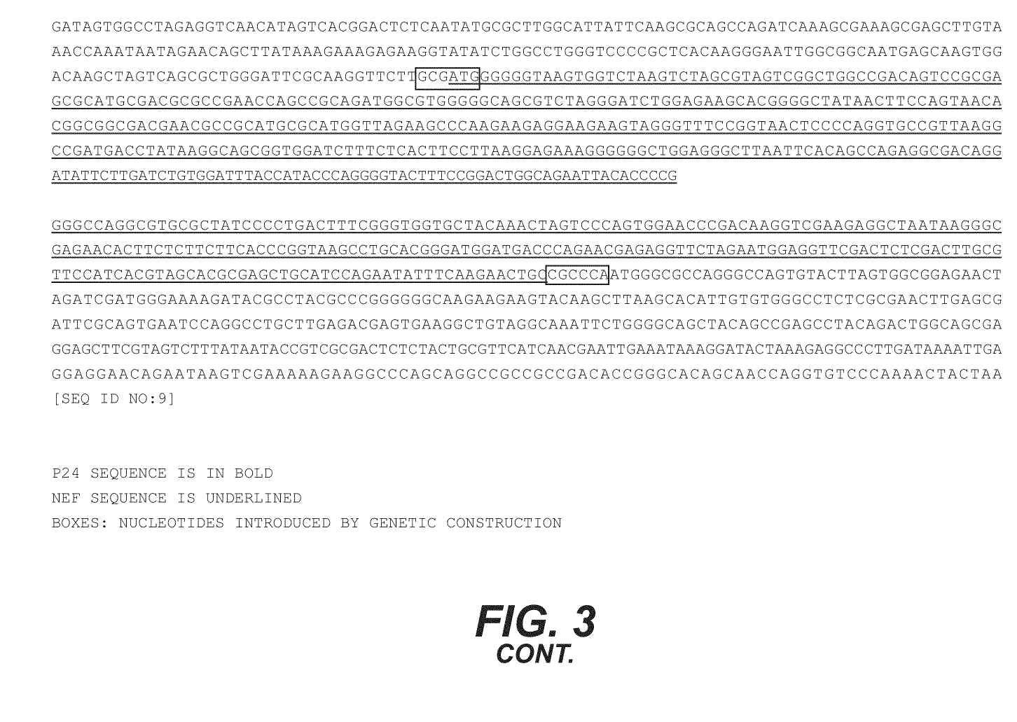

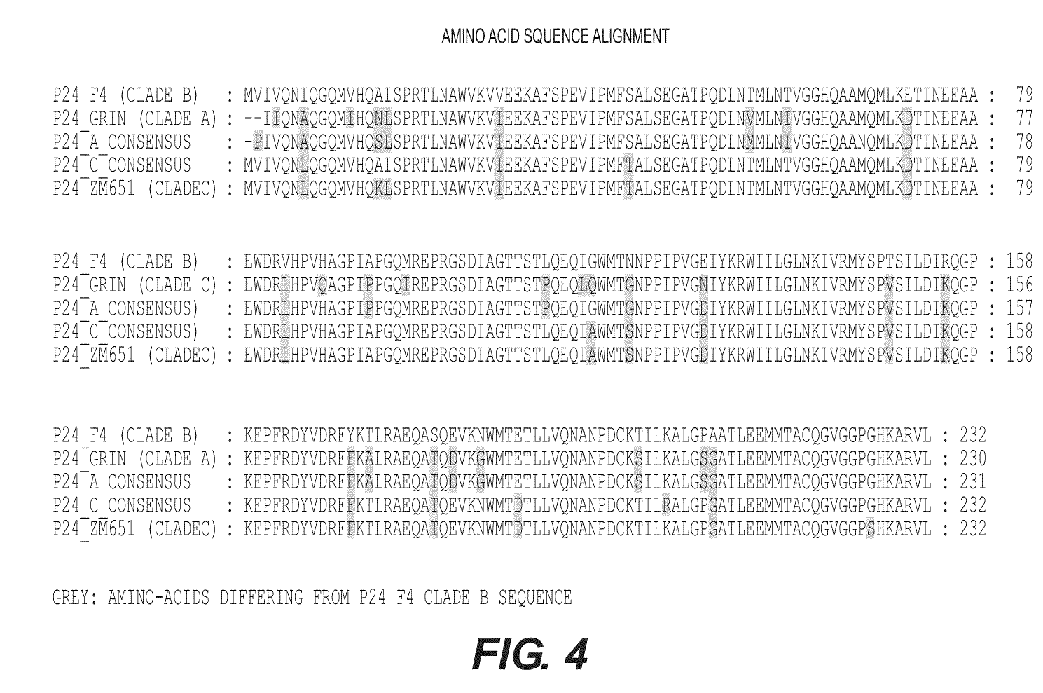

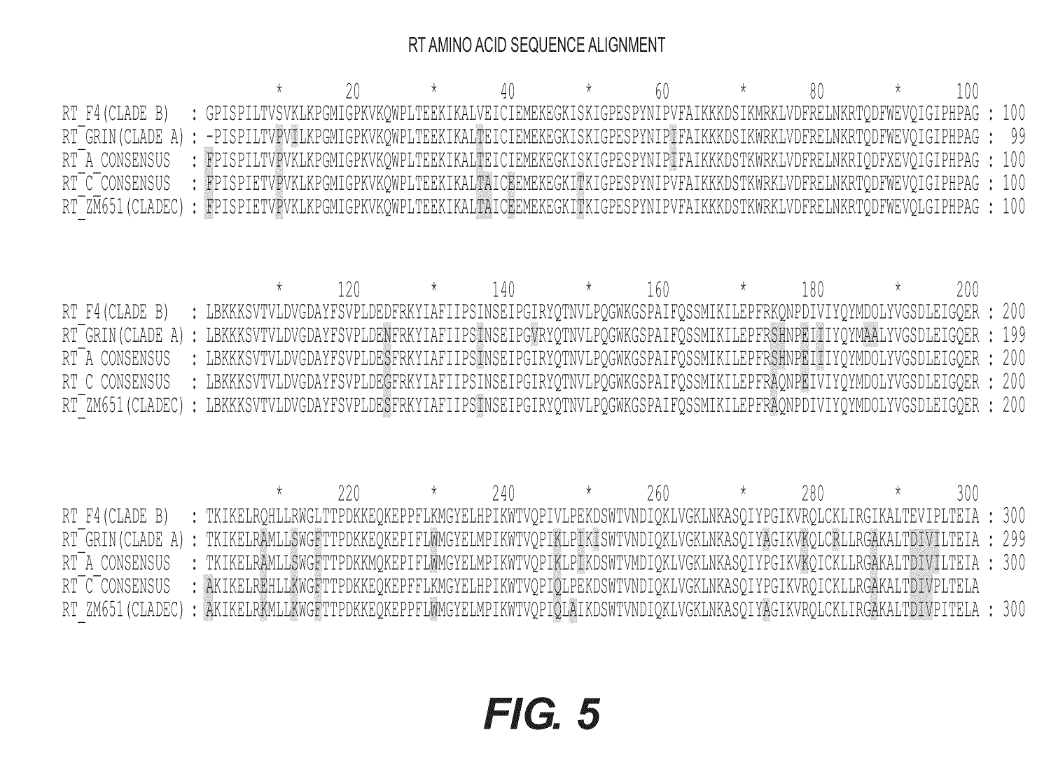

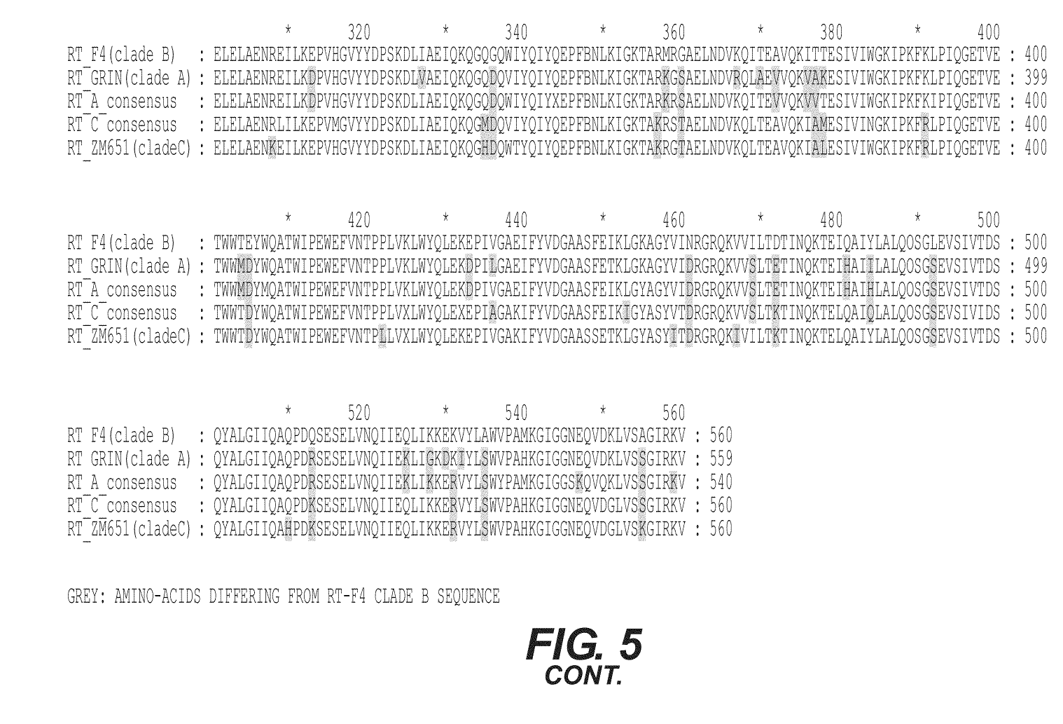

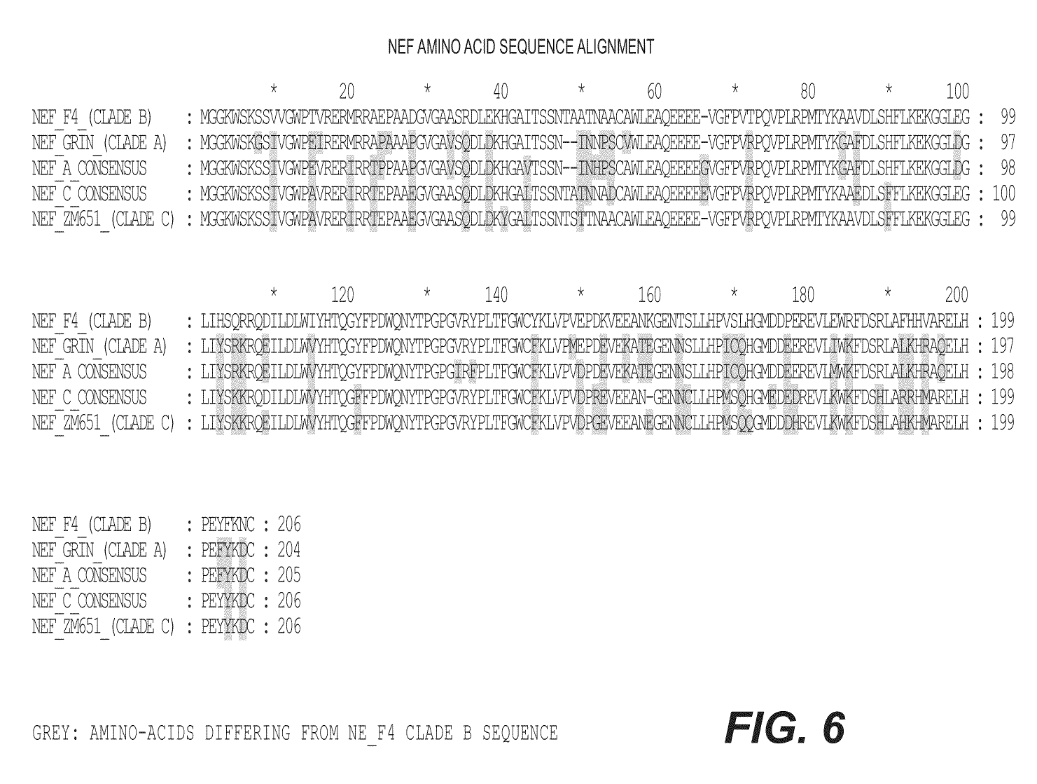

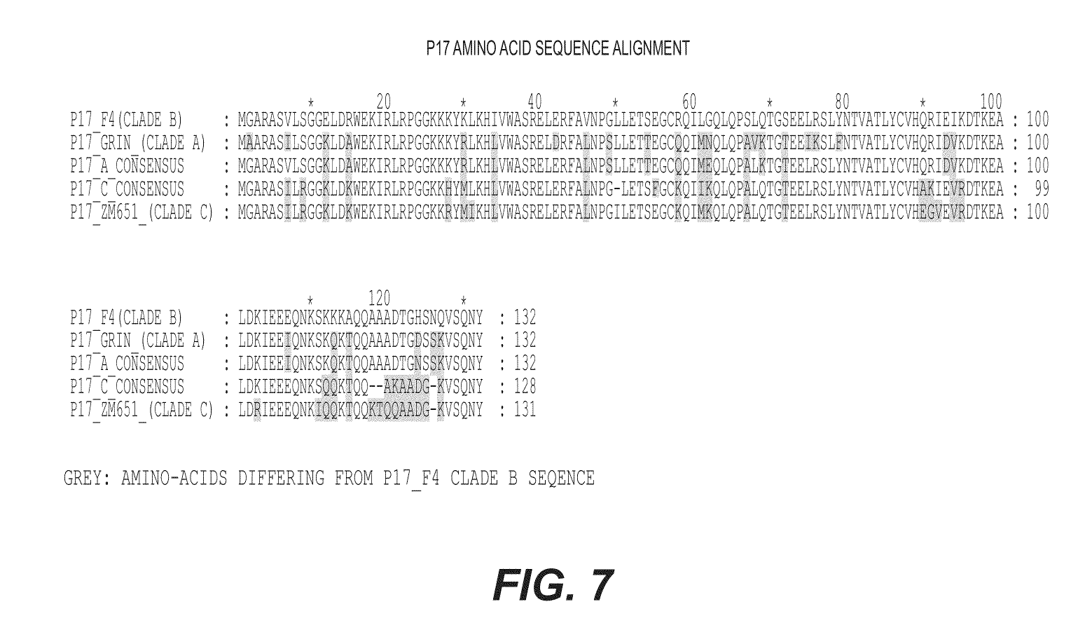

### DETAILED DESCRIPTION OF THE INVENTION

The present invention provides an immunogenic composition comprising
a. one or more polypeptides comprising one or more antigens selected from: Nef, Pol and/or Gag;
   wherein said one or more antigens are selected from one or more HIV-1 strains from one or more clades; and
b. an adjuvant that is a preferential inducer of a Th1 immune response,
   for use in the treatment or prevention of disease or infection by an HIV-1 strain from one or more clades different from the one or more HIV-1 clades in the immunogenic composition.

The inventors have found that an immunogenic composition comprising proteinaceous HIV-1 antigens from a strain of HIV-1 from one particular clade can induce strong CD4+ T cell responses to strains of HIV-1 from other, different HIV-1 clades. For example, HIV-1 antigens from a clade B strain of HIV-1 can induce cross-reactive immune responses to HIV-1 antigens from non-clade B strains of HIV, for example to HIV-1 antigens from clade A, clade C or recombinant clades such as clade CRF-01 (circulating recombinant form of clades A and E), among others, as well as to HIV-1 antigens from other, different clade B strains of HIV-1.

This cross-reactivity is in addition to the “homologous” immune response seen against HIV-1 antigens from the same strain of HIV-1. Accordingly, an immunogenic composition comprising HIV-1 antigens from an HIV-1 strain from clade B, for example, can be used to treat or prevent HIV-1 infection and disease caused by the same strain of HIV-1, a different strain of HIV-lfrom clade B or other, non-clade B strains of HIV, for example strains from clade A and/or clade C and/or clade CRF-01.

An immune response is generated to an antigen through the interaction of the antigen with the cells of the immune system. The resultant immune response may be broadly distinguished into two major streams: the innate immune response that is less diversified but independent in action, and the adaptive immune response with enormous diversity but strong dependency on innate immunity and hence limited autonomy.

Efficient host defense against invading pathogens is achieved through coordination of complex signalling networks that link the innate and adaptive immune systems. Protection against virus infections is predominantly mediated by adaptive immunity and by both humoral and cell-mediated immunity. The antiviral effect of humoral immunity is mediated through the generation of neutralizing antibodies capable of blocking virus entry/infection of the target cells. CD4+ and CD8+ T cells are the effector components of cell-mediated immunity, mediating their antiviral effect through the secretion of cytokines and the killing of virus-infected target cells.

A number of studies strongly support the protective role of HIV-1-specific T-cell responses in the control of virus replication and in the prevention of HIV-1-associated disease.

Upon interaction with antigen presented by antigen presenting cells such as dendritic cells (DCs), CD4+ T cells can differentiate into a variety of effector subsets, including classical Th1 cells and Th2 cells, Th17 cells, follicular helper T (Tfh) cells, and induced regulatory T (iTreg) cells. The differentiation decision is governed predominantly by the presence of cytokines and, to some extent, by the strength of the interaction between the antigen and the T cell antigen receptor.

Th1 cells are characterized by their production of IFN-γ and are involved in cellular immunity against intracellular microorganisms. IL-12, produced by innate immune cells, directs cells toward the Th1 cell differentiation program, as well as IFN-γ produced by both NK cells and T cells.

Th2 cells produce IL-4, IL-5, and IL-13 and are required for humoral immunity to control helminths and other extracellular pathogens. Th2 cell differentiation requires the action of GATA3 downstream of IL-4 and Stat6.

Th17 cells produce IL-17A, IL-17F, and IL-22 and play important roles in clearance of extracellular bacteria and fungi, especially at mucosal surfaces. Th17 cell differentiation requires retinoid related orphan receptor (ROR)gt, a transcription factor that is induced by TGF-β in combination with the proinflammatory cytokines IL-6, IL-21, and IL-23, all of which activate Stat3 phosphorylation.

Tfh cells are a subset of helper T cells that regulate the maturation of B cell responses. Differentiation of these cells requires the cytokine IL-21 and may be dependent on the transcription factor Bcl-6.

Tight regulation of effector T cell responses is required for effective control of infections and avoidance of autoimmune and immunopathological diseases.

As well as being known to play a central role in the immune control of many viral infections, virus-specific CD4+ T-cells are required for the induction and maintenance of functional CD8+ T-cells which, as discussed above, are known to play a role in controlling persistent viral infections, including infection by HIV-1.

The HIV-1 genome encodes a number of different proteins. Envelope proteins include gp120 and its precursor gp160, for example. Non-envelope proteins of HIV-1 include for example internal structural proteins such as the products of the gag and pol genes and other non-structural proteins such as Rev, Nef, Vif and Tat.

Since such CD4+ T-cell responses against the broadest possible spectrum of circulating HIV-1 strains are favourable, an HIV-1 vaccine desirably contains as many different CD4 epitopes as possible from various viral proteins. The viral antigens containing the highest number of conserved T-cell epitopes are Gag, Pol, and Nef.

The immunogenic compositions of the invention comprise one or more polypeptides comprising one or more of these antigens.

In an embodiment, the immunogenic composition of the invention comprises one or more polypeptides comprising Nef.

HIV-1 Nef is an early protein, i.e. it is expressed early in infection and in the absence of structural protein. The Nef gene encodes an early accessory HIV-1 protein which has been shown to possess several activities. For example, the Nef protein is known to cause the down regulation of CD4, the HIV-1 receptor, and MHC class 1 molecules from the cell surface, although the biological importance of these functions is debated. Additionally Nef interacts with the signal pathways of T cells and induces an active state, which in turn can promote more efficient gene expression. Some HIV-1 isolates have mutations in this region, which cause them not to encode functional protein and are severely compromised in their replication and pathogenesis in vivo. References to Nef are to full length Nef and to fragments, variants and derivatives of full length Nef. The term also includes polypeptides comprising Nef, including polypeptides comprising fragments, variants and derivatives of Nef.

In an embodiment, Nef is from an HIV-1 strain of clade A, B, C, D, E, F, G, H, J, K, or a circulating recombinant form of HIV-1 (CRF).

Conveniently, Nef is from an HIV-1 strain of clade B.

In an embodiment the immunogenic composition of the invention comprises one or more polypeptides comprising Pol.

The Pol gene encodes two proteins containing the two activities needed by the virus in early infection, the RT and the integrase protein needed for integration of viral DNA into cell DNA. The primary product of Pol is cleaved by the virion protease to yield the amino terminal RT peptide which contains activities necessary for DNA synthesis (RNA and DNA-dependent DNA polymerase activity as well as an RNase H function) and carboxy terminal integrase protein. RT is thus an example of a fragment of Pol. HIV-1 RT is a heterodimer of full-length RT (p66) and a cleavage product (p51) lacking the carboxy terminal RNase H domain, each of which are also examples of fragments of Pol.

References to Pol are to full length Pol and to fragments, variants and derivatives of full length Pol. The term also includes polypeptides comprising Pol, including polypeptides comprising fragments, variants and derivatives of Pol.

In an embodiment, Pol comprises the RT fragment. The RT fragment is an example of a fragment of Pol. References to RT are also to full length RT and to fragments, variants and derivatives of full length RT. The term also includes polypeptides comprising RT, including polypeptides comprising fragments, variants and derivatives of RT. In this manner, RT can comprise the p66 fragment, the p51 fragment and/or fragments, variants and derivatives of p66 and/or p51.

In an embodiment, Pol is from an HIV-1 strain of clade A, B, C, D, E, F, G, H, J, K, or a circulating recombinant form of HIV-1 (CRF)

Conveniently, Pol is from an HIV-1 strain of clade B.

In an embodiment the immunogenic composition of the invention comprises one or more polypeptides comprising Gag.

The Gag gene is translated as a precursor polyprotein that is cleaved by protease to yield products that include the matrix protein (p17), the capsid (p24), the nucleocapsid (p9), p6 and two space peptides, p2 and p1, all of which are examples of fragments of Gag.

The Gag gene gives rise to the 55-kilodalton (kD) Gag precursor protein, also called p55, which is expressed from the unspliced viral mRNA. During translation, the N terminus of p55 is myristoylated, triggering its association with the cytoplasmic aspect of cell membranes. The membrane-associated Gag polyprotein recruits two copies of the viral genomic RNA along with other viral and cellular proteins that triggers the budding of the viral particle from the surface of an infected cell. After budding, p55 is cleaved by the virally encoded protease (a product of the pol gene) during the process of viral maturation into four smaller proteins designated MA (matrix [p17]), CA (capsid [p24]), NC (nucleocapsid [p9]), and p6, all of which are examples of fragments of Gag.

The p17 (MA) polypeptide is from the N-terminal, myristoylated end of p55. Most MA molecules remain attached to the inner surface of the virion lipid bilayer, stabilizing the particle. A subset of MA is recruited inside the deeper layers of the virion where it becomes part of the complex which escorts the viral DNA to the nucleus. These MA molecules facilitate the nuclear transport of the viral genome because a karyophilic signal on MA is recognized by the cellular nuclear import machinery. This phenomenon allows HIV-1 to infect non-dividing cells.

The p24 (CA) protein forms the conical core of viral particles. Cyclophilin A has been demonstrated to interact with the p24 region of p55 leading to its incorporation into HIV-1 particles. The interaction between Gag and cyclophilin A is essential because the disruption of this interaction by cyclosporin A inhibits viral replication.

The NC region of Gag is responsible for specifically recognizing the so-called packaging signal of HIV-1. The packaging signal consists of four stem loop structures located near the 5′ end of the viral RNA, and is sufficient to mediate the incorporation of a heterologous RNA into HIV-1 virions. NC binds to the packaging signal through interactions mediated by two zinc-finger motifs. NC also facilitates reverse transcription.

The p6 polypeptide region mediates interactions between p55 Gag and the accessory protein Vpr, leading to the incorporation of Vpr into assembling virions. The p6 region also contains a so-called late domain which is required for the efficient release of budding virions from an infected cell.

In an embodiment, Gag is from an HIV-1 strain of clade A, B, C, D, E, F, G, H, J, K, or a circulating recombinant form of HIV-1 (CRF).

Conveniently, Gag is from an HIV-1 strain of clade B.

In an embodiment, Gag is p17. In such embodiment, p17 is from an HIV-1 strain of clade A, B, C, D, E, F, G, H, J, K, or a circulating recombinant form of HIV-1 (CRF). Conveniently, p17 is from an HIV-1 strain of clade B.

In an embodiment, Gag is p24. In such embodiment, p24 is from an HIV-1 strain of clade A, B, C, D, E, F, G, H, J, K, or a circulating recombinant form of HIV-1 (CRF). Conveniently, p24 is from an HIV-1 strain of clade B.

In an embodiment, Gag comprises both p17 and p24 either as separate protein antigen components or fused together.

Conveniently, p17 and p24 are fused together and are separated by a heterologous amino-acid sequence.

In the present invention, antigens described are full length antigens, for example, full length Nef, full length Pol, full length Gag. The invention also encompasses antigens that are not full length, including fragments or variants of the antigen, which may or may not correspond to full length. Suitably, fragments are immunogenic fragments and variants are immunogenic variants.

Typically, “fragments”, whether immunogenic or otherwise, contain a contiguous sequence of amino acids from the polypeptide comprising an HIV-1 antigen of which they are a fragment. Conveniently, the fragments contain at least 5 to 8 amino acids, at least 9 to 15 amino acids, at least 20, at least 50, or at least 100 contiguous amino acids from the polypeptide of which they are a fragment.

“Immunogenic fragments”, as used herein, will comprise at least one epitope of the antigen and display HIV-1 antigenicity. Such fragments are capable of inducing an immune response against the native antigen, either in isolation or when presented in a suitable construct, such as when fused to other HIV-1 epitopes or antigens, fused to a fusion partner which can be proteinaceous and/or immunogenic, or when presented on or in a carrier.

The term “variant”, as used herein, includes polypeptides that have been altered in a limited way compared to their non-variant counterparts. This includes point mutations which can change the properties of the polypeptide for example by improving expression in expression systems or removing undesirable activity including undesirable enzyme activity. However, the polypeptide variant comprising an HIV-1 antigen must remain sufficiently similar to the native polypeptide such that they retain the antigenic properties desirable in an immunogenic composition or vaccine and thus they remain capable of raising an immune response against the native antigen. Whether or not a particular variant raises such an immune response can be measured by a suitable immunological assay such as an ELISA (for antibody responses) or flow cytometry using suitable staining for cellular markers and cytokines (for cellular responses).

Conveniently, “variants” according to the present invention, comprise additions, deletions or substitutions of one or more amino acids. They encompass truncated antigens, where the C-terminus and/or the N-terminus of the antigen has been cleaved of one or more amino acids. Conveniently, “variants” include truncates or fragments wherein 1 to 5 amino acids, 6 to 10 amino acids, 11 to 15 amino acids, 16 to 20 amino acids, 21 to 25 amino acids or more than 25 amino acids are cleaved from the C-terminus and/or the N-terminus of the antigen

Variants of the invention can incorporate one or more deletions, additions or substitutions of one or more amino acids. Accordingly, a truncate of an antigen can additionally comprise deletions, additions or substitutions of one or more amino acids at a different part of the peptide.

Variants of the invention also comprise a polypeptide sequences that have 70, 80, 90, 95 or 98% identity with the polypeptide sequence of Nef, Pol and/or Gag.

In an embodiment of the invention, the immunogenic compositions comprise two polypeptides comprising one or more antigens, three polypeptides comprising one or more antigens, four polypeptides comprising one or more antigens or five or more polypeptides comprising one or more antigens.

Each of Nef, Pol and/or Gag can be present in the immunogenic composition more than once. For example, an immunogenic composition of the invention can comprise two or more polypeptides comprising Nef, two or more polypeptides comprising Pol and/or two or more polypeptides comprising Gag.

In a further embodiment, each of the one or more polypeptides can comprise one of Nef, Pol and/or Gag, two of Nef, Pol and/or Gag, three of Nef, Pol and/or Gag, four of Nef, Pol and/or Gag, and so forth. If more than one polypeptide is present in the composition, each polypeptide can comprise the same number and/or composition of antigens or each polypeptide can comprise a different number and/or composition of antigens. If there are three or more polypeptides in the composition, two or more polypeptides can comprise the same number and/or composition of antigens while the remaining polypeptide(s) can comprise a different number and/or composition of antigens.

It has been well documented that the polypeptide sequences for these antigens are well conserved across different strains, including across strains from different clades of HIV-1.

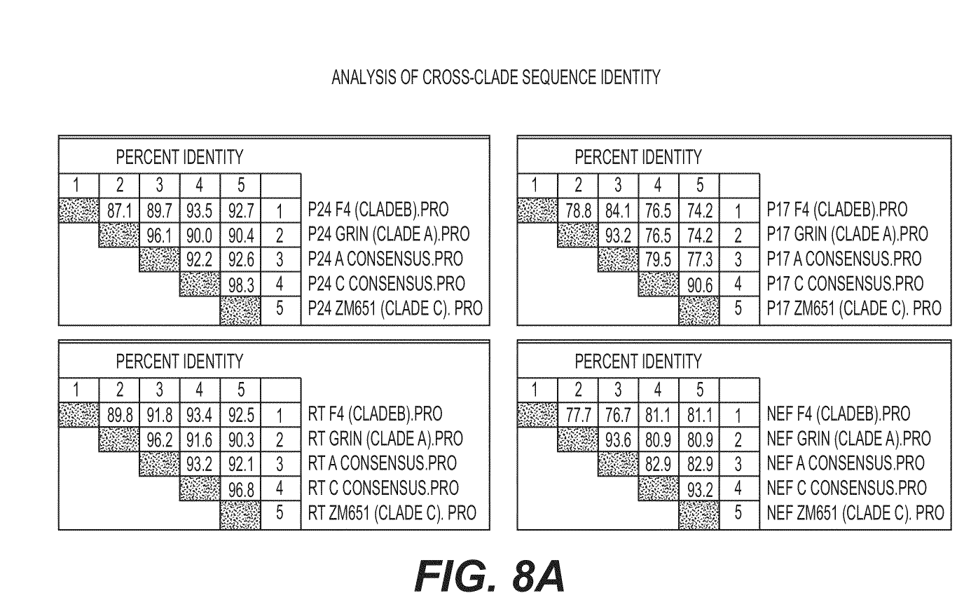

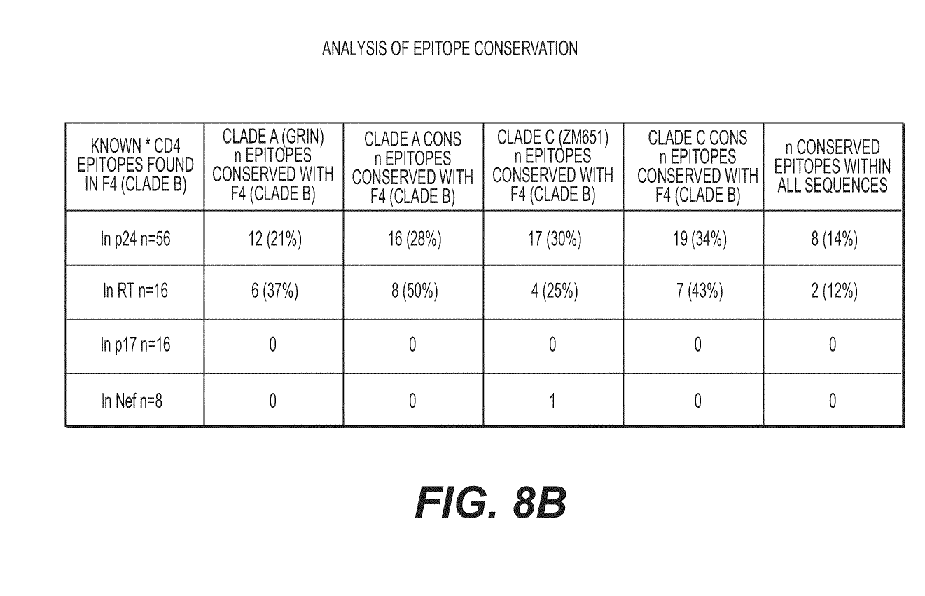

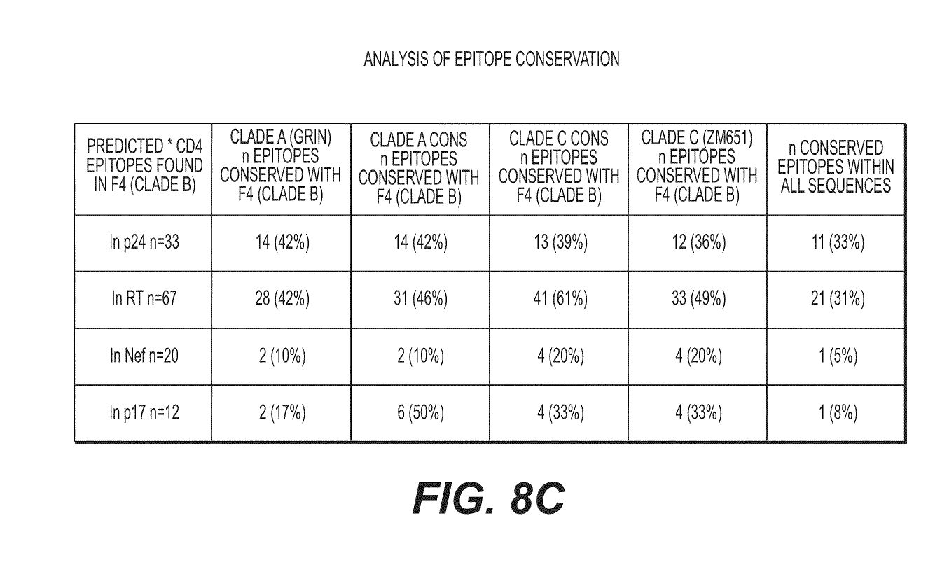

However, an analysis of CD4+ T cell epitope conservation for HIV-1 antigens across different clades shows that while the sequences for these antigens can be relatively well conserved across the different clades, CD4+ T-cell epitopes appear to be surprisingly less well conserved (see FIG. 8 for analysis).

The analysis looked at “known” CD4+ T cell epitopes and “predicted” CD4+ T cell epitopes. Epitopes were “predicted” using three softwares: SVMHC (www-bs.informatik.uni-tuebingen.de/SVMHC), ProPred (www.imtech.res.in/raghava/propred/) and a GSKbio program (Tepitope). To be selected as a potential “epitope”, a 9-mer peptide had to be predicted by the three programs as an epitope for a given HLA allele, among the 17 alleles that are the most represented in the Caucasian population. The number of predicted CD4+ T-cell epitopes conserved among the sequences was then calculated.

Despite the relatively small degree of observed epitope conservation among the clades studied, the inventors surprisingly found that there is a much higher degree of cross-reactivity than would be expected.

Cross-reactivity is herein taken to mean the ability of immune responses induced by an immunogenic composition of the invention to recognize strains of HIV-1 from clades that are not represented in the immunogenic composition. For example, an immunogenic composition of the invention comprising a polypeptide comprising an antigen from a strain of HIV-1 from clade B is considered cross-reactive if the HIV-specific immune response, such as HIV-specific CD4+ T cell response, induced by the composition reacted with different strains of HIV-1 not in the composition, for example, with a strain of HIV-1 from a clade other than clade B.

A reaction by the composition-induced immune response to strains of HIV-1 from different clades will depend on the type of immune response induced. For CD4 specific immune responses this can include the secretion of relevant cytokines. CD8 specific immune responses can include the induction of cytolytic activity and/or the secretion of relevant cytokines. Antibody specific responses can include the induction of neutralizing antibodies, for example.

In the present invention, it is preferred that the immune response induced by strains of HIV-1 from clades not represented in the immunogenic composition of the invention is a CD4 specific immune response.

Cross-reactive immune responses can be directed against immunogenic regions on the polypeptide, i.e. epitopes, that are conserved in sequence across different virus strains from different HIV-1 clades, or they can be directed against epitopes that tolerate a certain degree of sequence variation without abrogating immune recognition.

Cross-reactivity can be measured by means of the magnitude of a given immune response and/or by means of the percentage of responders.

When considering the magnitude of an immune response, this can be expressed in terms of the magnitude of a given immune response induced by polypeptides from the same clade or clades as the clade(s) in the immunogenic composition versus an immune response induced by polypeptides from a clade that is not represented in the immunogenic composition.

In order to analyse the magnitude of an immune response, the polypeptides comprising antigens in the immunogenic composition can be used in immunological assays, for example, in the form of proteins for the analysis of antibody responses or in the form of sets of overlapping synthetic peptides for the analysis of T cell responses. Examples of suitable immunological assays are well known to a person skilled in the art and are described in the Examples below.

The magnitude of an immune response can also be expressed as the titer (or concentration) of antigen-specific antibodies induced by the immunogenic composition as determined by an appropriate serological test. The magnitude of a T cell response can be expressed as the frequency (or number) of antigen-specific cells induced by the immunogenic composition among the total population of T cells, which can be monitored by cytokine production.

The magnitude of an immune response, for example a cross-reactive immune response, can be influenced by the number of wholly or partially conserved epitopes as well as the dominance of recognised epitopes.

For example, a high magnitude immune response can result from recognition of a large number of relatively weak conserved epitopes or the recognition of a few dominant epitopes.

Conveniently, the level of cross-reactivity observed is up to 10%, up to 15%, up to 20%, up to 25%, up to 30%, up to 35%, up to 40%, up to 45%, up to 50%, up to 55%, up to 60%, up to 65% up to 70%, up to 80%, up to 90% or up to 100% of antigen-specific cells induced by the immunogenic composition among the total population of T cells or titer (or concentration) of antigen-specific antibodies induced by the immunogenic composition.

When measuring cross-reactivity in terms of the percentage of responders to the strains of HIV-1 from different clades, the number or percentage of vaccinated individuals that show a positive response in an immunological assay after subsequent challenge can be measured. A responder can respond to one or more epitopes on an antigen. A responder can also respond to one or more polypeptides in an immunogenic composition of the invention and/or to one or more antigens in an immunogenic composition of the invention.

Immunological assays and serological tests that can be used to analyse the percentage of responders or the magnitude of an immune response are known in the art. Examples of such assays are known to a person skilled in the art and are described below in the Examples section.

Preferably, the level of cross-reactivity observed is up to 10%, up to 15%, up to 20%, up to 25%, up to 30%, up to 35%, up to 40%, up to 45%, up to 50%, up to 55%, up to 60%, up to 65% up to 70%, up to 80%, up to 90% or up to 100% of subjects in a sample are responders.

No matter which method of measurement is used, complete cross-reactivity, for example 100% cross-reactivity, against a strain of HIV-1 from a clade not represented in the immunogenic composition is not required in the present invention.

The immunogenic composition of the invention comprises an adjuvant that is a preferential inducer of a Th1 immune response.

Adjuvants are described in general in Vaccine Design—the Subunit and Adjuvant Approach, edited by Powell and Newman, Plenum Press, New York, 1995, incorporated herein by reference.

An adjuvant, an example of an immunostimulant, refers to the components in an immunogenic composition that enhance or potentiate a specific immune response (antibody and/or cell-mediated) to an antigen.

Adjuvants can induce immune responses of the Th1-type and Th-2 type response. Th1-type cytokines (e.g., IFN-γ, IL-2, and IL-12) tend to favour the induction of cell-mediated immune response to a given antigen, while Th-2 type cytokines (e.g., IL-4, IL-5, 11-6, IL-10) tend to favour the induction of humoral immune responses to the antigen.

In the present invention, the adjuvant is a preferential inducer of a Th1 immune response.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4+ T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p145-173, incorporated herein by reference). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of 11-4, IL-5, IL-6, IL-10. Suitable adjuvant systems which promote a predominantly Th1 response include: Monophosphoryl lipid A or a derivative thereof (or detoxified lipid A in general—see for instance WO2005107798), particularly 3-de-O-acylated monophosphoryl lipid A (3D-MPL) (for its preparation see GB 2220211 A); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen [Thoelen et al. Vaccine (1998) 16:708-14; EP 689454-B 1, each incorporated herein by reference].

An enhanced system involves the combination of a monophosphoryl lipid A and a saponin derivative, particularly the combination of QS21 and 3D-MPL as disclosed in WO 94/00153 incorporated herein by reference, or a less reactogenic composition where the QS21 is quenched with cholesterol as disclosed in WO 96/33739, incorporated herein by reference. A particularly potent adjuvant formulation involving QS21, 3D-MPL and tocopherol in an oil in water emulsion is described in WO 95/17210, incorporated herein by reference. In one embodiment the immunogenic composition additionally comprises a saponin, which can be QS21. The formulation can also comprise an oil in water emulsion and tocopherol (WO 95/17210, incorporated herein by reference).

In an embodiment of the invention, the adjuvant comprises one or more components selected from an immunologically active saponin fraction and/or a lipopolysaccharide and/or an immunostimulatory oligonucleotides.

In an embodiment, the adjuvant comprises an immunologically active saponin fraction and a lipopolysaccharide.

Conveniently, the immunologically active saponin fraction is QS21 and/or the lipopolysaccharide is a lipid A derivative. Suitably, the lipid A derivative is 3D-MPL.

Suitable adjuvants are combinations of 3D-MPL and QS21 (EP 0 671 948 B1, incorporated herein by reference), oil in water emulsions comprising 3D-MPL and QS21 (WO 95/17210, WO 98/56414, each incorporated herein by reference), or 3D-MPL formulated with other carriers (EP 0 689 454 B1, incorporated herein by reference).

3D-MPL is available from GlaxoSmithKline Biologicals North America and primarily promotes CD4+ T cell responses with an IFN-γ (Th1) phenotype. It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3D-MPL is used. Small particle 3D-MPL has a particle size such that it can be sterile-filtered through a 0.22 μm filter. Such preparations are described in WO 94/21292, incorporated herein by reference.

Another suitable adjuvant for use in the present invention is Quil A and its derivatives. Quil A is a saponin preparation isolated from the South American tree Quilaja Saponaria Molina and was first described as having adjuvant activity by Dalsgaard et al. in 1974 (“Saponin adjuvants”, Archiv. für die gesamte Virusforschung, Vol. 44, Springer Verlag, Berlin, p243-254, incorporated herein by reference). Purified fragments of Quil A have been isolated by HPLC which retain adjuvant activity without the toxicity associated with Quil A (EP 0 362 278, incorporated herein by reference), for example QS7 and QS21 (also known as QA7 and QA21). QS21 is a natural saponin derived from the bark of Quillaja saponaria Molina which induces CD8+ cytotoxic T cells (CTLs), Th1 cells and a predominant IgG2a antibody response and is a preferred saponin in the context of the present invention.

Particular formulations of QS21 have been described which are particularly suitable, these formulations further comprise a sterol (WO96/33739, incorporated herein by reference). The saponins forming part of the present invention can be separate in the form of micelles, mixed micelles (preferentially, but not exclusively with bile salts) or can be in the form of ISCOM matrices (EP 0 109 942 B1, incorporated herein by reference), liposomes or related colloidal structures such as worm-like or ring-like multimeric complexes or lipidic/layered structures and lamellae when formulated with cholesterol and lipid, or in the form of an oil in water emulsion (for example as in WO 95/17210, incorporated herein by reference). The saponins can be associated with a metallic salt, such as aluminium hydroxide or aluminium phosphate (WO 98/15287, incorporated herein by reference).

An enhanced system involves the combination of a monophosphoryl lipid A (or detoxified lipid A) and a saponin derivative, particularly the combination of QS21 and 3D-MPL as disclosed in WO 94/00153, incorporated herein by reference, or a less reactogenic composition where the QS21 is quenched with cholesterol as disclosed in WO 96/33739, incorporated herein by reference. A particularly potent adjuvant formulation involving tocopherol with or without QS21 and/or 3D-MPL in an oil in water emulsion is described in WO 95/17210, incorporated herein by reference.

In an embodiment, the adjuvant comprises a sterol, which can suitably be cholesterol. Suitable sterols, for instance cholesterol, act to reduce the reactogenicity of the composition while maintaining the adjuvant effect of the saponin.

In an embodiment of the invention, the adjuvant comprises a liposome carrier.

In an embodiment, the adjuvant comprises a saponin and a sterol with a ratio of saponin:sterol from 1:1 to 1:100 (w/w). Conveniently, the ratio of saponin:sterol is from 1:1 to 1:10 (w/w) or the ratio of saponin:sterol is from 1:1 to 1:5 (w/w).

In an embodiment, the adjuvant comprises a saponin and a lipopolysaccharide with a ratio of saponin:lipopolysaccharide of 1:1.

Conveniently, the adjuvant comprises a lipopolysaccharide and said lipopolysaccharide is present at an amount of 1-60 μg per dose. Suitably, the lipopolysaccharide is present at an amount of 50 μg per dose, 25 μg per dose, 10 μg per dose or 5 μg per dose.

Conveniently, the adjuvant comprises a saponin and said saponin is present at an amount of 1-60 μg per dose. Suitably, the saponin is present at an amount of 50 μg per dose, 25 μg per dose, 10 μg per dose or 5 μg per dose.

In an embodiment, the adjuvant comprises (per 0.5 mL dose) 0.025-2.5, 0.05-1.5, 0.075-0.75, 0.1-0.3, or 0.125-0.25 mg (e.g. 0.2-0.3, 0.1-0.15, 0.25 or 0.125 mg) sterol (for instance cholesterol).

In an embodiment, the adjuvant comprises (per 0.5 mL dose) 5-60, 10-50, or 20-30 μg (e.g. 5-15, 40-50, 10, 20, 30, 40 or 50 mg) lipid A derivative (for instance 3D-MPL).

In an embodiment, the adjuvant comprises (per 0.5 mL dose) 5-60, 10-50, or 20-30 μg (e.g. 5-15, 40-50, 10, 20, 30, 40 or 50 μg) saponin (for instance QS21).

In an embodiment of the invention, the adjuvant comprises an oil-in-water emulsion.

Conveniently, the oil-in-water emulsion comprises squalene and/or alpha tocopherol. Suitably, the oil-in-water emulsion is a metabolisable oil-in-water emulsion. In particular, the oil-in-water emulsion suitably comprises an emulsifier such as Tween 80.

The adjuvant can conveniently comprise a saponin and a lipopolysaccharide. In particular, the adjuvant can comprise a saponin and a lipopolysaccharide at a ratio of saponin:lipopolysaccharide in the range 1:10 to 10:1 (w/w).

The adjuvant can conveniently comprise a saponin and a sterol. In particular, the adjuvant can comprise a saponin and a sterol at a ratio of saponin:sterol in the range of 1:1 to 1:20 (w/w).

The adjuvant can conveniently comprise a saponin and a metabolisable oil. In particular, the adjuvant can comprise a saponin and a metabolisable oil at a ratio of metabolisable oil:saponin is in the range from 1:1 to 250:1 (w/w).

The adjuvant can conveniently comprise alpha tocopherol.

In an embodiment, the adjuvant comprises (per 0.5 mL dose) 0.5-15, 1-13, 2-11, 4-8, or 5-6 mg (e.g. 2-3, 5-6, or 10-11 mg) metabolisable oil (such as squalene).

In an embodiment, the adjuvant comprises (per 0.5 mL dose) 0.1-10, 0.3-8, 0.6-6, 0.9-5, 1-4, or 2-3 mg (e.g. 0.9-1.1, 2-3 or 4-5 mg) emulsifier (such as Tween 80).

In an embodiment, the adjuvant comprises (per 0.5 mL dose) 0.5-20, 1-15, 2-12, 4-10, 5-7 mg (e.g. 11-13, 5-6, or 2-3 mg) tocol (such as alpha tocopherol).

In an embodiment, the adjuvant comprises (per 0.5 mL dose) 5-60, 10-50, or 20-30 μg (e.g. 5-15, 40-50, 10, 20, 30, 40 or 50 mg) lipid A derivative (for instance 3D-MPL).

In an embodiment, the adjuvant comprises (per 0.5 mL dose) 0.025-2.5, 0.05-1.5, 0.075-0.75, 0.1-0.3, or 0.125-0.25 mg (e.g. 0.2-0.3, 0.1-0.15, 0.25 or 0.125 mg) sterol (for instance cholesterol).

In an embodiment, the adjuvant comprises (per 0.5 mL dose) 5-60, 10-50, or 20-30 μg (e.g. 5-15, 40-50, 10, 20, 30, 40 or 50 μg) saponin (for instance QS21).

In another embodiment, the adjuvant comprises a metal salt and a lipid A derivative.

Such adjuvant systems of interest include those based on aluminium salts in conjunction with the lipopolysaccharide 3-de-O-acylated monophosphoryl lipid A. The antigen and 3-de-O-acylated monophosphoryl lipid A can be co-adsorbed to the same metallic salt particles or can be adsorbed to distinct metallic salt particles.

Suitably, the adjuvant comprises (per 0.5 mL dose) 100-750, 200-500, or 300-400 mg Al, for instance as aluminium phosphate. In such embodiment, the adjuvant comprises (per 0.5 mL dose) 5-60, 10-50, or 20-30 mg (e.g. 5-15, 40-50, 10, 20, 30, 40 or 50 mg) lipid A derivative (for instance 3D-MPL).

In an embodiment of the invention, the adjuvant comprises an immunostimulatory oligonucleotide comprising a CpG motif.

Immunostimulatory oligonucleotides can be used in the immunogenic composition of the invention. The preferred oligonucleotides for use in adjuvants or immunogenic compositions of the present invention are CpG containing oligonucleotides, preferably containing two or more dinucleotide CpG motifs separated by at least three, more preferably at least six or more nucleotides. A CpG motif is a Cytosine nucleotide followed by a Guanine nucleotide. The CpG oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204, each incorporated herein by reference.

Examples of preferred oligonucleotides have the following sequences. The sequences preferably contain phosphorothioate modified internucleotide linkages.

OLIGO 1(SEQ ID NO: 1):
TCC ATG ACG TTC CTG ACG TT
OLIGO 2 (SEQ ID NO: 2):
TCT CCC AGC GTG CGC CAT
OLIGO 3(SEQ ID NO: 3):
ACC GAT GAC GTC GCC GGT GAC GGC ACC ACG
OLIGO 4 (SEQ ID NO: 4):
TCG TCG TTT TGT CGT TTT GTC GTT
OLIGO 5 (SEQ ID NO: 5):
TCC ATG ACG TTC CTG ATG CT
OLIGO 6 (SEQ ID NO: 6):
TCG ACG TTT TCG GCG CGC GCC G

Alternative CpG oligonucleotides can comprise the preferred sequences above in that they have inconsequential deletions or additions thereto.

The CpG oligonucleotides utilised in the present invention can be synthesized by any method known in the art (for example see EP 468520, incorporated herein by reference). Conveniently, such oligonucleotides can be synthesized utilising an automated synthesizer.

According to an aspect of the invention, the immunogenic composition of the invention is for use in the treatment or prevention of disease or infection by HIV-1 strains from one or more clades different from the one or more HIV-1 clades in the immunogenic composition.

Additionally, in an embodiment of the invention, the immunogenic composition of the invention is for use in the treatment or prevention of disease or infection by HIV-1 strains from one or more clades that are in the immunogenic composition.

Accordingly, the immunogenic composition is capable of inducing homologous immune responses to the clades represented therein as well as inducing cross-reactive immune responses to clades that are not covered by the strains of antigen present in the composition. The immunogenic compositions are thus capable of broadly treating or preventing disease or infection by multiple HIV-1 strains from multiple clades.

Conveniently, in all embodiments of the invention, the one or more HIV-1 clades in the immunogenic composition are selected from clade A, B, C, D, E, F, G, H, J, K, or a circulating recombinant form of HIV-1 (CRF). In particular, the one or more HIV-1 clades in the immunogenic composition is clade B.

Similarly conveniently, in all embodiments of the invention, the one or more clades different from the one or more HIV-1 clades in the immunogenic composition are selected from clade A, B, C, D, E, F, G, H, J, K, or a circulating recombinant form of HIV-1 (CRF). In particular, the one or more clades different from the one or more HIV-1 clades in the immunogenic composition are selected from clade A or C. Suitably, the one or more clades different from the one or more HIV-1 clades in the immunogenic composition are clade A and C.

These HIV-1 clades vary in prevalence around the world. Accordingly, an immunogenic composition of the invention need not be limited in its suitable territory of use and can be used to treat and prevent HIV-1 infection and disease around the world

In an embodiment, the immunogenic composition of the invention is for use in inducing a humoral immune response against HIV-1 strains from one or more clades different from the one or more HIV-1 clades in the immunogenic composition. Conveniently in such embodiment, humoral immune responses can also be induced against HIV-1 strains from one or more clades in the immunogenic composition.

In the present invention, humoral immune responses were characterized by strong antigen-specific antibody titers. Immune responses were detected by ELISA-based assays. The induction of strong humoral responses indicates the broad immunogenicity of the immunogenic compositions of the invention.

In an embodiment, the immunogenic composition of the invention is for use in conferring a long term non-progressor status on an individual infected with an HIV-1 strain from one or more clades different from the one or more HIV-1 clades in the immunogenic composition. Conveniently in such embodiment, a long term non-progressor status can also be conferred on an individual infected with an HIV-1 strain from one or more clades in the immunogenic composition.

Long term non-progressors (LTNPs) are HIV-1 infected patients who are capable of controlling HIV-1 infection, thus preventing the progression of disease. Such a status is often associated with the presence of polyfunctional and proliferation-competent HIV-1-specific CD4+ T-cells.

In an embodiment, the immunogenic composition of the invention is for use in inducing multiple-cytokine-producing antiviral CD4+ T cells against HIV-1 strains from one or more clades different from the one or more HIV-1 clades in the immunogenic composition. Conveniently in such embodiment, multiple-cytokine-producing antiviral CD4+ T cells can be induced against HIV-1 strains from one or more clades in the immunogenic composition.

Conveniently, the CD4+ T cells produce two or more of IL2, IFN-γ and TNFα. Such cells can be considered polyfunctional. In this respect, the CD4+ T cells produce IL2 and IFN-γ or the CD4+ T cells produce IL2 and TNFα or the CD4+ T cells produce IFNγ and TNFα. Suitably, the CD4+ T cells produce IL2, IFNγ and TNFα. In an embodiment, the CD4+ T cells express CD40L.

The induction of CD4+ T cells that exhibit a polyfunctional phenotype is indicative of the broad immunogenicity of the immunogenic compositions of the invention. The presence of such polyfunctional CD4+ T cells is associated with control of HIV-1 infection and viremia, for instance, with non-progression of infection and/or disease in long term non-progressors.

In an embodiment, the immunogenic composition of the invention is for use in preventing progressive CD4+ T cell decline in an individual infected with an HIV-1 strain from one or more clades different from the one or more HIV-1 clades in the immunogenic composition. Conveniently in such embodiment, progressive CD4+ T cell decline is also prevented in an individual infected with an HIV-1 strain from one or more clades in the immunogenic composition.

In an embodiment, the immunogenic composition of the invention is for use in reducing or eliminating viral reservoirs in an individual infected with an HIV-1 strain from one or more clades different from the one or more HIV-1 clades in the immunogenic composition. Conveniently in such embodiment, viral reservoirs can be reduced or eliminated in an individual infected with an HIV-1 strain from one or more clades in the immunogenic composition.

In an embodiment, the immunogenic composition of the invention is for use in eliciting high and long-lasting numbers of HIV-1-specific polyfunctional CD4+ T-cells in an individual infected with an HIV-1 strain from one or more clades different from the one or more HIV-1 clades in the immunogenic composition. Conveniently in such embodiment, high and long-lasting numbers of HIV-1-specific polyfunctional CD4+ T-cells can be elicited in an individual infected with an HIV-1 strain from one or more clades in the immunogenic composition.

In an embodiment, the immunogenic composition of the invention is for use in controlling or reducing viremia in an individual infected with an HIV-1 strain from one or more clades different from the one or more HIV-1 clades in the immunogenic composition. Conveniently in such embodiment, viremia can be controlled or reduced in an individual infected with an HIV-1 strain from one or more clades in the immunogenic composition.

In an embodiment, the immunogenic composition of the invention is for use in inducing long term memory of an antiviral immune response against HIV-1 strains from one or more clades different from the one or more HIV-1 clades in the immunogenic composition. Conveniently, long term memory of an antiviral immune response can be induced against HIV-1 strains from one or more clades in the immunogenic composition.

Suitably, the antiviral immune response comprises the induction of persistent antiviral CD4+ T cells. Such T cells are induced in response to the antigens from clades represented in the immunogenic composition as well as clades that are not represented in the immunogenic composition, and thus are cross-reactive immune responses according to the present invention.

Conveniently, the CD4+ T cells persist for at least 6 months. By “persist”, it is meant that the CD4+ T cells are capable of existing for an extended period of time in a phenotypic state equivalent to that when they are initially induced. For instance, if the CD4+ T cells release two or more specific cytokines when first induced, persisting CD4+ T cells will still exhibit the same cytokine profile after an extended period, for example after at least 6 months.

Suitably, the CD4+ T cells persist for 6 to 24 months or 9 to 18 months, for instance for 12 months.

Accordingly, the present invention also provides for methods for the treatment or prophylaxis of HIV infection comprising administering the immunogenic composition to a subject and inducing an immune response in the subject against an HIV-1 strain from one or more clades different from the one or more HIV-1 clades in the immunogenic composition. In other embodiments, the present invention provides methods for the use of the composition as described herein.

Fusion proteins comprising one or more of the antigens which can be present in the immunogenic composition of the invention have been disclosed in WO2006/013106, incorporated herein by reference. The antigens Pol, Nef, Gag and variants and fragments thereof have previously been selected for inclusion in a fusion protein for use in an immunogenic composition because they are considered to be relatively well conserved across different strains of HIV, and thus is more likely to cross-react with antigens from different strains of HIV, than less well conserved antigens. However, the incorporation of these antigens into fusion proteins may introduce unpredictable complications because the antigens therein do not correspond to native proteins. Accordingly, fusion proteins are not straightforward to produce and cannot be presumed to behave as the native protein would.

In an embodiment of the invention, two, three, four or more of the antigens in the immunogenic composition are fused to form a fusion protein.

Conveniently, Gag is fused to Pol or Pol is fused to Gag, Pol is fused to Nef or Nef is fused to Pol, and/or Nef is fused to Gag or Gag is fused to Nef.

Suitably, in a fusion protein of the invention, Gag is p17 and/or p24, and/or Pol is RT

In particular, it is convenient that the antigens in the immunogenic composition are fused to form a fusion protein comprising Nef, RT, p17 and p24 in any order. Suitably, the antigens are fused to form a fusion protein comprising p24-RT-Nef-p17. Such a fusion protein is known as F4 and is described in the Examples.

The antigens in a fusion protein can be fused directly to each other or by means of a linker. Such linker can comprise a heterologous amino acid sequence comprising one or more amino acids.

The antigens in the fusion can be from the same strain of HIV, can be from different strains within the same HIV-1 clade or can be from different strains from different HIV-1 clades.

In one embodiment, the antigens in the fusion protein are from HIV-1 strains from two, three or four different HIV-1 clades. Alternatively, all of the antigens in the fusion protein are from an HIV-1 strain or strains from the same HIV-1 clade.

The peptides according to the invention can be combined with other antigens. In particular, this can include HIV-1 env proteins or fragments or variants thereof. Preferred forms of env are gp120, gp140 and gp160. The env can be for example the envelope protein described in WO 00/07631 from an HIV-1 clade B envelope clone known as R2, or fragments or variants thereof. The env can also be the gp120 clone known as W61.D, or fragments or variants thereof.

Thus the invention further provides an immunogenic composition according to the invention further comprising an HIV-1 env protein or fragment or variant thereof. For the sake of clarity, the meaning of the terms “fragment” and “variant” used here are as defined above.

In an embodiment, immunogenic compositions of the invention that comprise a fusion protein further comprise one or more unfused polypeptides comprising an antigen.

In one embodiment, the antigen in the unfused polypeptide is from a strain of HIV-1 from the same clade as at least one of the antigens in the fusion protein.

Alternatively, the antigen in the unfused polypeptide is from a strain of HIV-1 different from the one or more clades in the fusion protein.

In an embodiment, the unfused polypeptide comprises Env. For instance, the unfused polypeptide comprises one or more of gp120, gp140 or gp160.

The HIV-1 envelope glycoprotein gp120 is the viral protein that is used for attachment to the host cell. The gp120 protein is the principal target of neutralizing antibodies, but unfortunately the most immunogenic regions of the proteins (V3 loop) are also the most variable parts of the protein. The gp120 protein also contains epitopes that are recognized by cytotoxic T lymphocytes (CTL). These effector cells are able to eliminate virus-infected cells, and therefore constitute a second major antiviral immune mechanism. In contrast to the target regions of neutralizing antibodies some CTL epitopes appear to be relatively conserved among different HIV-1 strains. For this reason gp120 and gp160 can be useful antigenic components in vaccines that aim at eliciting cell-mediated immune responses (particularly CTL).

In tone embodiment, in the immunogenic composition of the invention, one of the one or more antigens in the immunogenic composition is from an HIV-1 from any one of the clades selected from A, B, C, D, E, F, G, H, J, K, or a circulating recombinant form of HIV-1 (CRF). When more than one antigen is present in the immunogenic composition, then all antigens can be from the same clade.

In an embodiment, in the immunogenic composition of the invention, one of the one or more antigens in the immunogenic composition is from an HIV-1 strain from clade B.

For example in certain embodiments, when two antigens are present in the immunogenic composition, both antigens are from an HIV-1 strain from clade B, or when three antigens are present in the immunogenic composition, all three antigens are from an HIV-1 strain from clade B, or when four antigens are present in the immunogenic composition, all four antigens are from an HIV-1 strain from clade B, and so forth.

Alternatively, in the immunogenic composition of the invention, one of the one or more antigens in the immunogenic composition is from an HIV-1 strain from clade C.

For example in certain embodiments, when two antigens are present in the immunogenic composition, both antigens are from an HIV-1 strain from clade C, or when three antigens are present in the immunogenic composition, all three antigens are from an HIV-1 strain from clade C, or when four antigens are present in the immunogenic composition, all four antigens are from an HIV-1 strain from clade C, and so forth.

The immunogenic compositions, or vaccines, of the present invention will contain an immunoprotective or immunotherapeutic quantity of the polypeptide and can be prepared by conventional techniques.

In an embodiment, the total amount of each antigen in a single dose of the immunogenic composition is 0.5-25 μg, 2-20 μg, 5-15 μg, or around 10 μg.

Suitably, the total amount of fusion protein in a single dose of the immunogenic composition is 10 μg and/or the total amount of unfused polypeptide in a single dose of the immunogenic composition is 20 μg.

In an embodiment, the total amount of all antigens in a single dose of the immunogenic composition is 0.5-50 μg, 2-40 μg, 5-30 μg, 7-20 μg or around 30 μg, around 20 μg or around 10 μg.

The amount of protein in a dose of the immunogenic composition is selected as an amount which induces an immune response without significant, adverse side effects in typical recipients. Such amount will vary depending upon which specific immunogen is employed and the dosing or vaccination regimen that is selected. An optimal amount for a particular immunogenic composition can be ascertained by standard studies involving observation of relevant immune responses in subjects.

Administration of the pharmaceutical composition can take the form of one or of more than one individual dose, for example as repeat doses of the same polypeptide containing composition, or in a heterologous “prime-boost” vaccination regime.

In an embodiment, the immunogenic composition of the invention is initially administered to a subject as two or three doses, wherein the doses are separated by a period of two weeks to three months, preferably one month.

Conveniently, the composition is administered to a subject (for instance as a booster) every 6-24, or 9-18 months, for instance annually. For instance, the composition is administered to a subject (for instance as a booster) at six month or 1 year intervals.

Suitably in this respect, subsequent administrations of the composition to the subject boost the immune response of earlier administrations of the composition to the same subject.

In an embodiment, the immunogenic composition of the invention is used as part of a prime-boost regimen for use in the treatment or prevention of disease or infection by HIV-1 strains from one or more clades different from the one or more HIV-1 clades in the immunogenic composition.

Conveniently, the composition is the priming dose. Alternatively, the composition is the boosting dose.

Suitably, two or more priming and/or boosting doses are administered.

A heterologous prime-boost regime uses administration of different forms of immunogenic composition or vaccine in the prime and the boost, each of which can itself include two or more administrations. The priming composition and the boosting composition will have at least one antigen in common, although it is not necessarily an identical form of the antigen, it can be a different form of the same antigen.

Prime boost immunisations according to the invention can be homologous prime-boost regimes or heterologous prime-boost regimes. Homologous prime-boost regimes utilize the same composition for prime and boost, for instance the immunogenic composition of the invention.

Heterologous prime-boost regimes can be performed with a combination of protein and DNA-based formulations. Such a strategy is considered to be effective in inducing broad immune responses. Adjuvanted protein vaccines induce mainly antibodies and CD4+ T cell immune responses, while delivery of DNA as a plasmid or a recombinant vector induces strong CD8+ T cell responses. Thus, the combination of protein and DNA vaccination can provide for a wide variety of immune responses. This is particularly relevant in the context of HIV, since neutralizing antibodies, CD4+ T cells and CD8+ T cells are thought to be important for the immune defense against HIV-1.

In one embodiment the DNA is delivered in a viral vector. For example, the viral vector may be derived from an adenovirus. In a further embodiment, the viral vector may be as described in US20100055166, which is hereby incorporated by reference for its disclosure of viral vectors and prime-boost methods. In another embodiment, the viral vector may be derived from a measles virus. In a further embodiment, the viral vector may be a recombinant measles vector as described in WO2010/023260, which is hereby incorporated by reference for its disclosure of viral vectors and prime-boost methods.

Accordingly, the present invention also provides for methods for the treatment or prophylaxis of HIV infection comprising administering a first composition to a subject and subsequently administering a second composition to the subject, wherein after administration, the subject has a detectable immune response in the subject against an HIV-1 strain from one or more clades different from the one or more HIV-1 clades in the immunogenic composition. In alternative embodiments, the method can be a homologous prime-boost regime or a heterologous prime-boost regime.

In a further aspect of the invention, the immunogenic composition of the invention is a vaccine composition.

Vaccine preparation is generally described in New Trends and Developments in Vaccines, edited by Voller et al., University Park Press, Baltimore, Md., U.S.A. 1978, incorporated herein by reference.

In a further aspect of the invention, there is provided use of the immunogenic composition or the vaccine of the invention in the manufacture of a medicament for the treatment or prevention of disease or infection by HIV-1 strains as described in all instances above.

In a further aspect of the invention, there is provided a method of treating or preventing HIV-1 disease or infection according to in all instances described above comprising the step of administering to a subject the immunogenic composition or the vaccine of the invention.

The terms “comprising”, “comprise” and “comprises” herein are intended by the inventors to be optionally substitutable with the terms “consisting of”, “consist of” and “consists of”, respectively, in every instance.

Embodiments herein relating to “immunogenic compositions” of the invention are also applicable to embodiments relating to “vaccines” of the invention, and vice versa.

In order that this invention may be better understood, the following examples are set forth. These examples are for purposes of illustration only, and are not to be construed as limiting the scope of the invention in any manner.

### Examples

The following examples and data illustrate the invention, but are not limiting upon the invention.

### 1. Adjuvant Preparations

A mixture of lipid (such as phosphatidylcholine either from egg-yolk or synthetic) and cholesterol and 3D-MPL in organic solvent was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was added to give a final cholesterol concentration of 10 mg/ml.

The liposomes have a size of approximately 100 nm and are referred to as SUV (for small unilamelar vesicles). The liposomes by themselves are stable over time and have no fusogenic capacity.

Sterile bulk of SUV was added to PBS. PBS composition was Na₂HPO₄:9 mM; KH₂PO₄:48 mM; NaCl:100 mM pH 6.1. QS21 in aqueous solution was added to the SUV. The final concentration of 3D-MPL and QS21 was 100 μg per ml for each. Between each addition of component, the intermediate product was stirred for 5 minutes. The pH was checked and adjusted if necessary to 6.1+/−0.1 with NaOH or HCl.

### 2. Preparation of HIV-1 Antigens

2.1 Construction and Expression of HIV-1 p24-RT-Nef-p17 Fusion (“F4”) and F4 Codon Optimized (co) (“F4Co”).

### 2.1.1 F4 Non-Codon-Optimised

HIV-1 gag p24 (capsid protein) and p17 (matrix protein), the reverse transcriptase and Nef proteins were expressed in E. coli B834 strain (B834 (DE3) is a methionine auxotroph parent of BL21 (DE3)), under the control of the bacteriophage T7 promoter (pET expression system).

They were expressed as a single fusion protein containing the complete sequence of the four proteins. Mature p24 coding sequence comes from HIV-1 BH10 molecular clone, mature p17 sequence and RT gene from HXB2 and Nef gene from the BRU isolate.

After induction, recombinant cells expressed significant levels of the p24-RT-Nef-p17 fusion that amounted to 10% of total protein.

When cells were grown and induced at 22° C., the p24-RT-Nef-p17 fusion protein was confined mainly to the soluble fraction of bacterial lysates (even after freezing/thawing). When grown at 30° C., around 30% of the recombinant protein was associated with the insoluble fraction.

The fusion protein p24-RT-Nef-p17 is made up of 1136 amino acids with a molecular mass of approximately 129 kDa. The full-length protein migrates to about 130 kDa on SDS gels. The protein has a theoretical isoelectric point (pI) of 7.96 based on its amino acid sequence, confirmed by 2D-gel electrophoresis.

### Details of the Recombinant Plasmid:

| | |
|---|---|
| name: | pRIT15436 (or lab name pET28b/p24-RT-Nef-p17) |
| | host vector: pET28b |
| | replicon: colE1 |
| selection: | kanamycin |
| promoter: | T7 |
| insert: | p24-RT-Nef-p17 fusion gene. |

### Details of the Recombinant Protein:

p24-RT-Nef-p17 fusion protein: 1136 amino acids.
N-term-p24: 232a.a.-hinge: 2a.a.-RT: 562a.a.-hinge: 2a.a.-Nef: 206a.a.-1-P17: 132a.a.-C-term

### Nucleotide and Amino-Acid Sequences:

Nucleotide sequence
[SEQ ID NO: 7]
atggttatcgtgcagaacatccaggggcaaatggtacatcaggccatatcacctagaactttaaatgcatgggtaaaagtagtaga
agagaaggctttcagcccagaagtaatacccatgttttcagcattatcagaaggagccaccccacaagatttaaacaccatgctaaa
cacagtggggggacatcaagcagccatgcaaatgttaaaagagaccatcaatgaggaagctgcagaatgggatagagtacatcc
agtgcatgcagggcctattgcaccaggccagatgagagaaccaaggggaagtgacatagcaggaactactagtacccttcaggaa
caaataggatggatgacaaataatccacctatcccagtaggagaaatttataaaagatggataatcctgggattaaataaaatagt
aagaatgtatagccctaccagcattctggacataagacaaggaccaaaagaaccttttagagactatgtagaccggttctataaaa
ctctaagagccgagcaagcttcacaggaggtaaaaaattggatgacagaaaccttgttggtccaaaatgcgaacccagattgtaag
actattttaaaagcattgggaccagcggctacactagaagaaatgatgacagcatgtcagggagtaggaggacccggccataagg

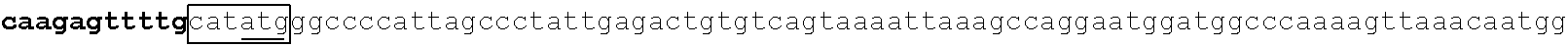

ccattgacagaagaaaaaataaaaagcattagtagaaatttgtacagagatggaaaaggaagggaaaatttcaaaaattgggcctgaaaatcc
atacaatactccagtatttgccataaagaaaaaagacagtactaaatggagaaaattagtagatttcagagaacttaataagagaactcaagac
ttctgggaagttcaattaggaataccacatcccgcagggttaaaaaagaaaaaatcagtaacagtactggatgtgggtgatgcatatttttcagt
tcccttagatgaagacttcaggaaatatactgcatttaccatacctagtataaacaatgagacaccagggattagatatcagtacaatgtgcttcc
acagggatggaaaggatcaccagcaatattccaaagtagcatgacaaaaatcttagagccttttagaaaacaaaatccagacatagttatctat
caatacatggatgatttgtatgtaggatctgacttagaaatagggcagcatagaacaaaaatagaggagctgagacaacatctgttgaggtgg
ggacttaccacaccagacaaaaaacatcagaaagaacctccattccttaaaatgggttatgaactccatcctgataaatggacagtacagcct
atagtgctgccagaaaaagacagctggactgtcaatgacatacagaagttagtggggaaattgaattgggcaagtcagatttacccagggatt
aaagtaaggcaattatgtaaactccttagaggaaccaaagcactaacagaagtaataccactaacagaagaagcagagctagaactggcag
aaaacagagagattctaaaagaaccagtacatggagtgtattatgacccatcaaaagacttaatagcagaaatacagaagcaggggcaagg
ccaatggacatatcaaatttatcaagagccatttaaaaatctgaaaacaggaaaatatgcaagaatgaggggtgcccacactaatgatgtaaa
acaattaacagaggcagtgcaaaaaataaccacagaaagcatagtaatatggggaaagactcctaaatttaaactgcccatacaaaaggaaa
catgggaaacatggtggacagagtattggcaagccacctggattcctgagtgggagtttgttaatacccctcctttagtgaaattatggtacca
gttagagaaagaacccatagtaggagcagaaaccttctatgtagatggggcagctaacagggagactaaattaggaaaagcaggatatgtt
actaatagaggaagacaaaaagttgtcaccctaactgacacaacaaatcagaagactgagttacaagcaatttatctagctttgcaggattcgg
gattagaagtaaacatagtaacagactcacaatatgcattaggaatcattcaggcacaaccagatcaaagtgaatcagagttagtcaatcaaat
aatagagcagttaataaaaaaggaaaaggtctatctggcatgggtaccagcacacaaaggaattggaggaaatgaacaagtagataaatta

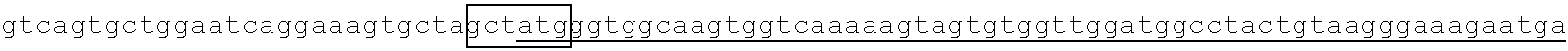

gacgagctgagccagcagcagatggggtgggagcagcatctcgagacctggaaaaacatggagcaatcacaagtagcaatacagcagct
accaatgctgcttgtgcctggctagaagcacaagaggaggaggaggtgggttttccagtcacacctcaggtacctttaagaccaatgact
tacaaggcagctgtagatcttagccactttttaaaagaaaaggggggactggaagggctaattcactcccaacgaagacaagatatccttgat
ctgtggatctaccacacacaaggctacttccctgattggcagaactacacaccagggccaggggtcagatatccactgacctttggatggtgc
tacaagctagtaccagttgagccagataaggtagaagaggccaataaaggagagaacaccagcttgttacaccctgtgagcctgcatggaa
tggatgaccctgagagagaagtgttagagtggaggtttgacagccgcctagcatttcatcacgtggcccgagagctgcatccggagtacttc

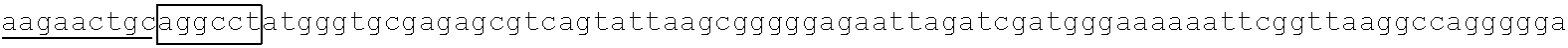

aagaaaaaatataaattaaaacatatagtatgggcaagcagggagctagaacgattcgcagttaatcctggcctgttagaaacatcagaagg
ctgtagacaaatactgggacagctacaaccatcccttcagacaggatcagaagaacttagatcattatataatacagtagcaaccctctattgtg
tgcatcaaaggatagagataaaagacaccaaggaagctttagacaagatagaggaagagcaaaacaaaagtaagaaaaaagcacagcaa
gcagcagctgacacaggacacagcaatcaggtcagccaaaattactaa
p24 sequence is in bold
Nef sequence is underlined
Boxes: nucleotides introduced by genetic construction
Amino-Acid sequence
[SEQ ID NO: 8]

| | |
|---|---|
| MVIVQNIQGQMVHQAISPRTLNAWVKVVEEKAFSPEVIPMFSALSEGATP | 50 |
| QDLNTMLNTVGGHQAAMQMLKETINEEAAEWDRVHPVHAGPIAPGQMREP | 100 |
| RGSDIAGTTSTLQEQIGWMTNNPPIPVGEIYKRWIILGLNKIVRMYSPTS | 150 |
| ILDIRQGPKEPFRDYVDRFYKTLRAEQASQEVKNWMTETLLVQNANPDCK | 200 |
| 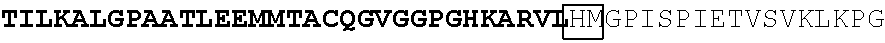 | 250 |
| MDGPKVKQWPLTEEKIKALVEICTEMEKEGKISKIGPENPYNTPVFAIKK | 300 |
| KDSTKWRKLVDFRELNKRTQDFWEVQLGIPHPAGLKKKKSVTVLDVGDAY | 350 |
| FSVPLDEDFRKYTAFTIPSINNETPGIRYQYNVLPQGWKGSPAIFQSSMT | 400 |
| KILEPFRKQNPDIVIYQYMDDLYVGSDLEIGQHRTKIEELRQHLLRWGLT | 450 |
| 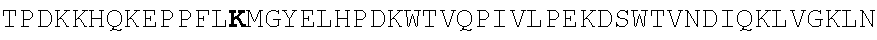 | 500 |
| WASQIYPGIKVRQLCKLLRGTKALTEVIPLTEEAELELAENREILKEPVH | 550 |
| GVYYDPSKDLIAEIQKQGQGQWTYQIYQEPFKNLKTGKYARMRGAHTNDV | 600 |
| KQLTEAVQKITTESIVIWGKTPKFKLPIQKETWETWWTEYWQATWIPEWE | 650 |
| FVNTPPLVKLWYQLEKEPIVGAETFYVDGAANRETKLGKAGYVTNRGRQK | 700 |
| VVTLTDTTNQKTELQAIYLALQDSGLEVNIVTDSQYALGIIQAQPDQSES | 750 |
| 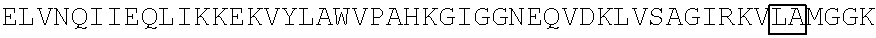 | 800 |
| WSKSSVVGWPTVRERMRRAEPAADGVGAASRDLEKHGAITSSNTAATNAA | 850 |
| CAWLEAQEEEEVGFPVTPQVPLRPMTYKAAVDLSHFLKEKGGLEGLIHSQ | 900 |
| RRQDILDLWIYHTQGYFPDWQNYTPGPGVRYPLTFGWCYKLVPVEPDKVE | 950 |
| EANKGENTSLLHPVSLHGMDDPEREVLEWRFDSRLAFHHVARELHPEYFK | 1000 |
| 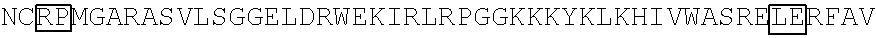 | 1050 |
| NPGLLETSEGCRQILGQLQPSLQTGSEELRSLYNTVATLYCVHQRIEIKD | 1100 |
| TKEALDKIEEEQNKSKKKAQQAAADTGHSNQVSQNY | 1136 |

P24 sequence: amino-acids 1-232 (in bold)
RT sequence: amino-acids 235-795
Nef sequence: amino-acids 798-1002
P17 sequence: amino-acids 1005-1136
Boxes: amino-acids introduced by genetic construction
K (Lysine): instead of Tryptophan (W). Mutation introduced to remover enzyme activity.

### Expression of the Recombinant Protein:

In pET plasmid, the target gene (p24-RT-Nef-p17) is under control of the strong bacteriophage T7 promoter. This promoter is not recognized by E. coli RNA polymerase and is dependent on a source of T7 RNA polymerase in the host cell. B834 (DE3) host cell contains a chromosomal copy of the T7 RNA polymerase gene under lacUV5 control and expression is induced by the addition of IPTG to the bacterial culture.

Pre-cultures were grown, in shake flasks, at 37° C. to mid-log phase (A620:0.6) and then stored at 4° C. overnight (to avoid stationary phase cultures). Cultures were grown in LBT medium supplemented with 1% glucose and 50 μg/mlkanamycin. Addition of glucose to the growth medium has the advantage to reduce the basal recombinant protein expression (avoiding cAMP mediated derepression of lacUV5 promoter).

Ten ml of cultures stored overnight at 4° C. were used to inoculate 200 ml of LBT medium (without glucose) containing kanamycin. Cultures were grown at 30° C. and 22° C. and when O.D.620 reached 0.6, IPTG was added (1 mM final). Cultures were incubated for further 3, 5 and 18 hours (overnight). Samples were collected before and after 3, 5 and 18 hours induction.

Extract preparation was as follows:

Cell pellets were suspended in breaking buffer* (at a theoretical O.D. of 10) and disrupted by four passages in French press (at 20.000 psi or 1250 bars). Crude extracts (T) were centrifuged at 20.000g for 30 min to separate the soluble (S) and insoluble (P) fractions.
*Breaking buffer: 50 mM Tris-HCL pH 8.0, 1 mM EDTA, 1 mM DTT+protease inhibitors cocktail (Complete/Boerhinger).

### SDS-PAGE and Western Blot Analysis:

Fractions corresponding to insoluble pellet (P), supernatant (S) and crude extract (T) were run on 10% reducing SDS-PAGE. p24-RT-Nef-p17 recombinant protein was detected by Coomassie blue staining and on Western blot (WB).
Coomassie staining: p24-RT-Nef-p17 protein appears as:
one band at ±130 kDa (fitting with calculated MW)
MW theoretical: 128.970 Daltons
MW apparent: 130 kDa

### Western Blot Analysis:

$$\begin{matrix}Reagents = & \text{-} Monoclonal antibody to RT ( p 66 / p 51 ) \\ & Purchased from ABI ( Advanced Biotechnologies ) \\ & dilution \text{:} 1 / 5000 \\ & \text{-} Alkaline phosphatase \text{-} conjugate anti \text{-} mouse antibody \\ & dilution \text{:} 1 / 7500\end{matrix}$$

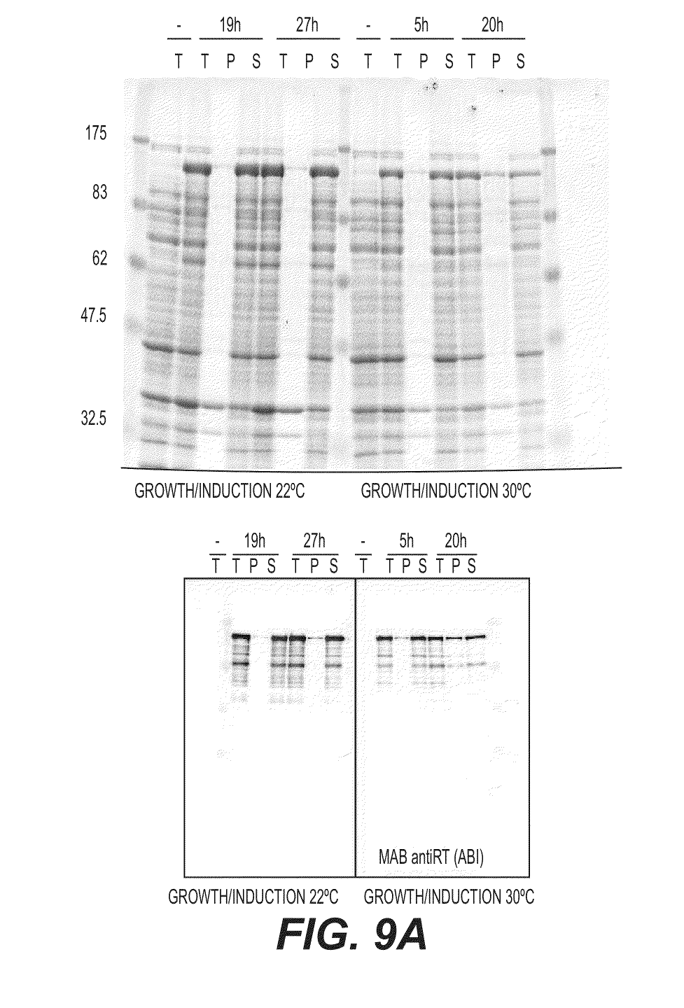

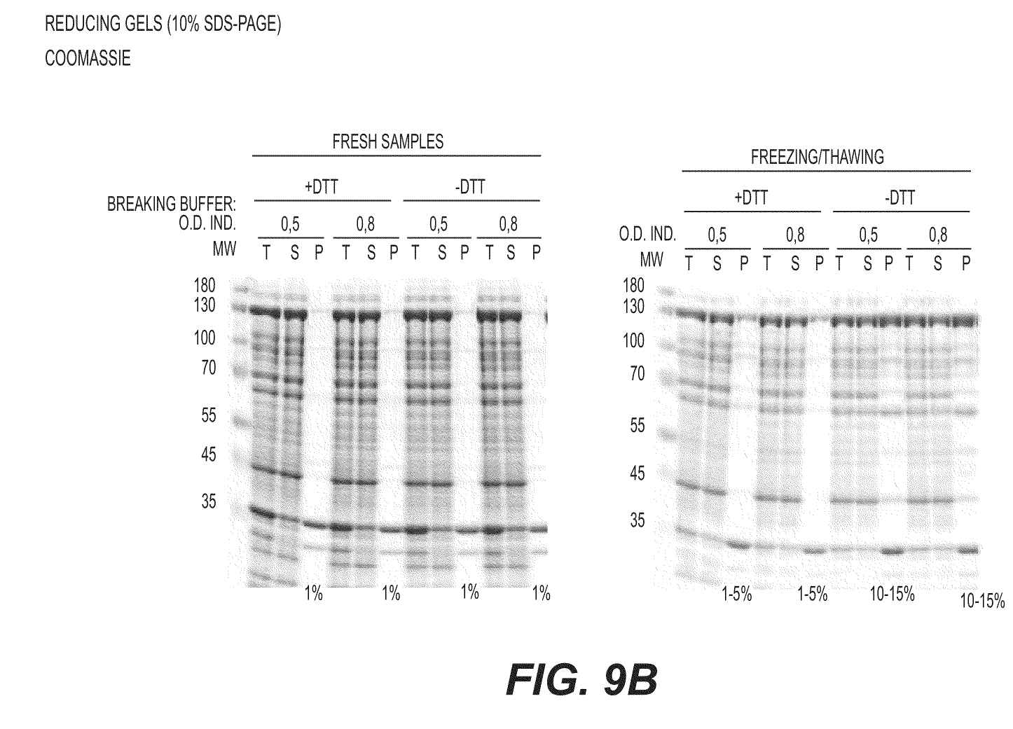

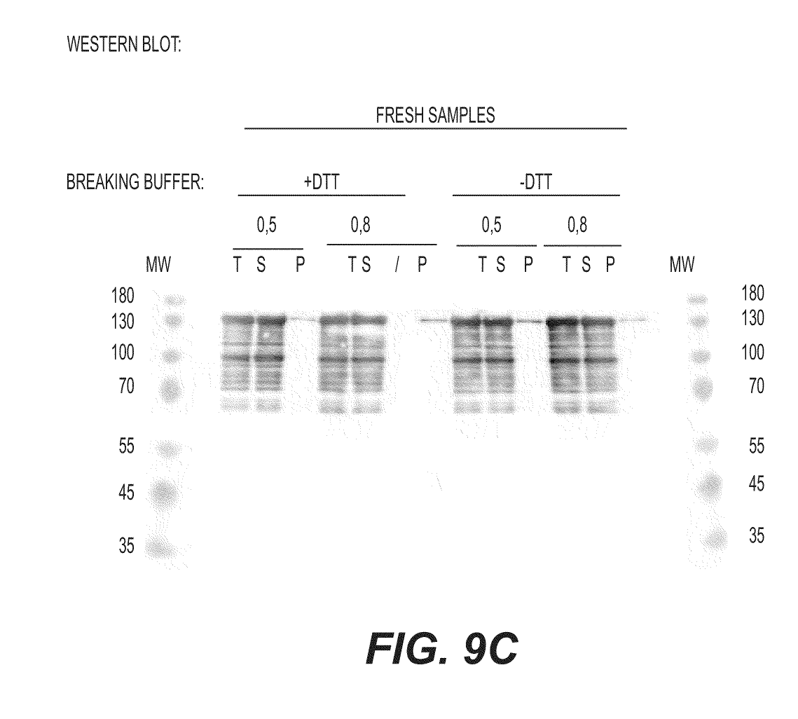

Expression level:—Very strong p24-RT-Nef-p17 specific band after 20 h induction at 22° C., representing up to 10% of total protein (See FIG. 9A).

### Recombinant Protein “Solubility”:

“Fresh” cellular extracts (T,S,P fractions): With growth/induction at 22° C./20 h, almost all p24-RT-Nef-p17 fusion protein is recovered in the soluble fraction of cellular extract (FIG. 9A). With growth/induction at 30° C./20 h, around 30% of p24-RT-Nef-p17 protein is associated with the insoluble fraction (FIG. 9A).

### “Freezing/Thawing” (S2, P2 Fractions):

Soluble (S1) fraction (20 h induction at 22° C.) conserved at −20° C. Thawed and centrifuged at 20.000g/30 min:S2 and P2 (resuspended in 1/10 vol.)
Breaking buffer with DTT: almost all p24-RT-Nef-p17 fusion protein still soluble (only 1-5% precipitated) (see FIG. 9B)
Breaking buffer without DTT: 85-90% of p24-RT-Nef-p17 still soluble (FIG. 9B)
The F4 protein was purified using the purification method identified below.

### 2.1.2 F4 Codon-Optimised

The following polynucleotide sequence is codon optimized such that the codon usage resembles the codon usage in a highly expressed gene in E. coli. The amino acid sequence is identical to that given above for F4 non-codon optimized.

Nucleotide sequence for F4co:
[SEQ ID NO: 9]
atggtcattgttcagaacatacagggccaaatggtccaccaggcaattagtccgcgaactcttaatgcatgggtgaaggtcgtggag
gaaaaggcattctccccggaggtcattccgatgttttctgcgctatctgagggcgcaacgccgcaagaccttaataccatgcttaaca
cggtaggcgggcaccaagccgctatgcaaatgctaaaagagactataaacgaagaggccgccgaatgggatcgagtgcacccgg
tgcacgccggcccaattgcaccaggccagatgcgcgagccgcgcgggtctgatattgcaggaactacgtctacccttcaggagcag
attgggtggatgactaacaatccaccaatcccggtcggagagatctataagaggtggatcatactgggactaaacaagatagtccg
catgtattctccgacttctatactggatatacgccaaggcccaaaggagccgttcagggactatgtcgaccgattctataagacccttc
gcgcagagcaggcatcccaggaggtcaaaaattggatgacagaaactcttttggtgcagaatgcgaatccggattgtaaaacaatt
ttaaaggctctaggaccggccgcaacgctagaagagatgatgacggcttgtcagggagtcggtggaccggggcataaagcccgcg

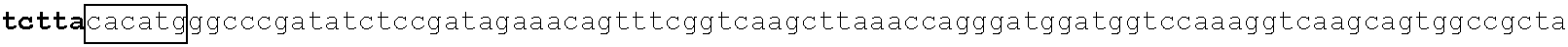

acggaagagaagattaaggcgctcgtagagatttgtactgaaatggagaaggaaggcaagataagcaagatcgggccagagaacccgta
caatacaccggtatttgcaataaagaaaaaggattcaacaaaatggcgaaagcttgtagattttagggaactaaacaagcgaacccaagactt
ttgggaagtccaactagggatcccacatccagccggtctaaagaagaagaaatcggtcacagtcctggatgtaggagacgcatattttagtgt
accgcttgatgaggacttccgaaagtatactgcgtttactataccgagcataaacaatgaaacgccaggcattcgctatcagtacaacgtgctc
ccgcagggctggaaggggtctccggcgatatttcagagctgtatgacaaaaatacttgaaccattccgaaagcagaatccggatattgtaattt
accaatacatggacgatctctatgtgggctcggatctagaaattgggcagcatcgcactaagattgaggaactgaggcaacatctgcttcgat
ggggcctcactactcccgacaagaagcaccagaaggagccgccgttcctaaagatgggctacgagcttcatccggacaagtggacagtac
agccgatagtgctgcccgaaaaggattcttggaccgtaaatgatattcagaaactagtcggcaagcttaactgggcctctcagatttacccag
gcattaaggtccgacagctttgcaagctactgaggggaactaaggctctaacagaggtcatcccattaacggaggaagcagagcttgagct
ggcagagaatcgcgaaattcttaaggagccggtgcacggggtatactacgacccctccaaggaccttatagccgagatccagaagcaggg
gcagggccaatggacgtaccagatatatcaagaaccgtttaagaatctgaagactgggaagtacgcgcgcatgcgaggggctcatactaat
gatgtaaagcaacttacggaagcagtacaaaagattactactgagtctattgtgatatggggcaagaccccaaagttcaagctgcccatacag
aaggaaacatgggaaacatggtggactgaatattggcaagctacctggattccagaatgggaatttgtcaacacgccgccacttgttaagcttt
ggtaccagcttgaaaaggagccgatagtaggggcagagaccttctatgtcgatggcgccgcgaatcgcgaaacgaagctaggcaaggcg
ggatacgtgactaataggggccgccaaaaggtgtaacccttacggataccaccaatcagaagactgaactacaagcgatttaccttgcactt
caggatagtggcctagaggtcaacatagtcacggactctcaatatgcgcttggcattattcaagcgcagccagatcaaagcgaaagcgagct
tgtaaaccaaataatagaacagcttataaagaaagagaaggtatatctggcctgggtccccgctcacaagggaattggcggcaatgagcaa

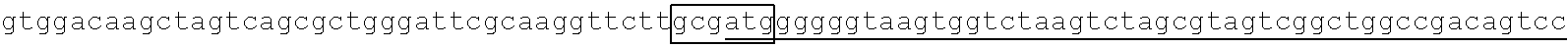

gcgagcgcatgcgacgcgccgaaccagccgcagatggcgtgggggcagcgtctagggatctggagaagcacggggctataacttccag
taacacggcggcgacgaacgccgcatgcgcatggttagaagcccaagaagaggaagaagtagggtttccggtaactccccaggtgccgtt
aaggccgatgacctataaggcagcggtggatctttctcacttccttaaggagaaaggggggctggagggcttaattcacagccagaggcga
caggatattcttgatctgtggatttaccatacccaggggtactttccggactggcagaattacaccccggggccaggcgtgcgctatcccctg
actttcgggtggtgctacaaactagtcccagtggaacccgacaaggtcgaagaggctaataagggcgagaacacttctcttcttcacccggt
aagcctgcacgggatggatgacccagaacgagaggttctagaatggaggttcgactctcgacttgcgttccatcacgtagcacgcgagctg

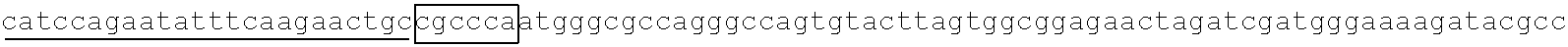

tacgcccggggggcaagaagaagtacaagcttaagcacattgtgtgggcctctcgcgaacttgagcgattcgcagtgaatccaggcctgctt
gagacgagtgaaggctgtaggcaaattctggggcagctacagccgagcctacagactggcagcgaggagcttcgtagtctttataataccg
tcgcgactctctactgcgttcatcaacgaattgaaataaaggatactaaagaggcccttgataaaattgaggaggaacagaataagtcgaaaa
agaaggcccagcaggccgccgccgacaccgggcacagcaaccaggtgtcccaaaactactaa
p24 sequence is in bold
Nef sequence is underlined
Boxes: nucleotides introduced by genetic construction

The procedures used in relation to F4 non-codon optimized were applied for the codon-optimised sequence.

### Details of the Recombinant Plasmid:

name: pRIT15513 (lab name: pET28b/p24-RT-Nef-p17)
host vector: pET28b
replicon: colE1
selection: kanamycin
promoter: T7
insert: p24-RT-Nef-p17 fusion gene, codon-optimized

The F4 codon-optimised gene was expressed in E. coli BLR(DE3) cells, a recA⁻ derivative of B834(DE3) strain. RecA mutation prevents the putative production of lambda phages.

Pre-cultures were grown, in shake flasks, at 37° C. to mid-log phase (A₆₂₀:0.6) and then stored at 4° C. overnight (to avoid stationary phase cultures).

Cultures were grown in LBT medium supplemented with 1% glucose and 50 μg/mlkanamycin. Addition of glucose to the growth medium has the advantage to reduce the basal recombinant protein expression (avoiding cAMP mediated derepression of lacUV5 promoter).

Ten ml of cultures stored overnight at 4° C. were used to inoculate 200 ml of LBT medium (without glucose) containing kanamycin. Cultures were grown at 37° C. and when O.D.₆₂₀ reached 0.6, IPTG was added (1 mM final). Cultures were incubated for a further 19 hours (overnight), at 22° C. Samples were collected before and after 19 hours induction.

Extract preparation was as follows:

Cell pellets were resuspended in sample buffer (at a theoretical O.D. of 10), boiled and directly loaded on SDS-PAGE.

### SDS-PAGE and Western Blot Analysis:

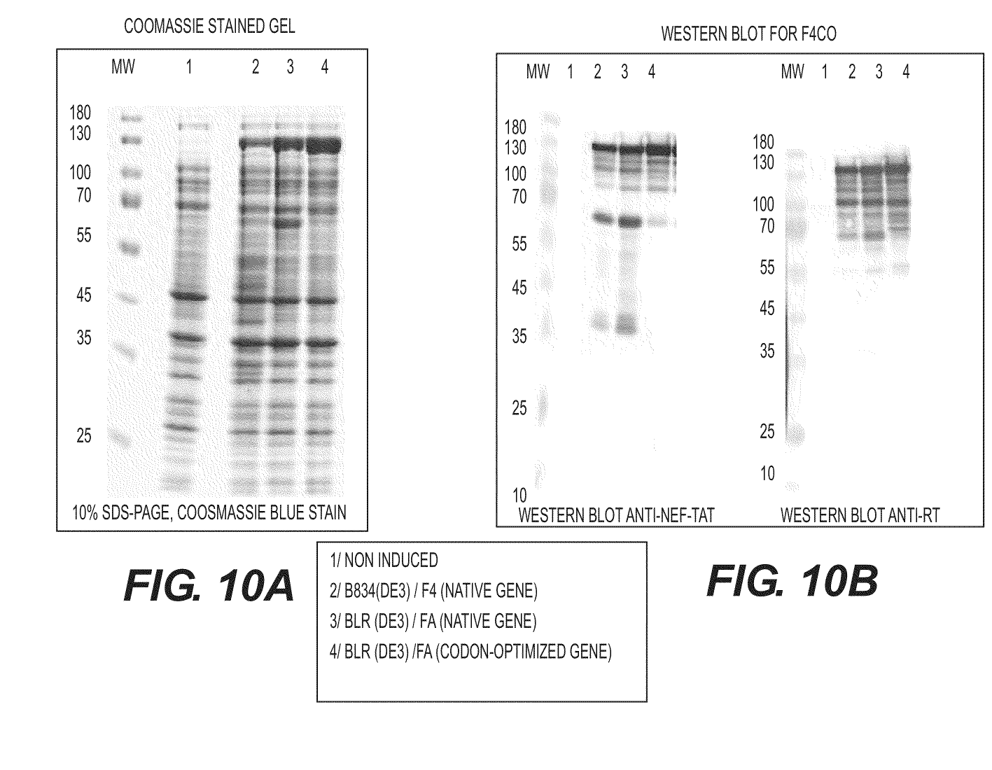

Crude extracts samples were run on 10% reducing SDS-PAGE.
p24-RT-Nef-p17 recombinant protein is detected by Coomassie blue staining (FIG. 10) and on Western blot.
Coomassie staining: p24-RT-Nef-p17 protein appears as:
   one band at ±130 kDa (fitting with calculated MW)
   MW theoretical: 128.967 Daltons
   MW apparent: 130 kDa

Western Blot Analysis:

$$\begin{matrix}Reagents = & \text{-} Rabbit polyclonal anti RT ( rabbit PO 3 L 16 ) \\ & dilution \text{:} 1 / 10.000 \\ & \text{-} Rabbit polyclonal anti NEF - Tat ( rabbit 388 ) \\ & dilution \text{:} 1 / 10.000 \\ & Alkaline phosphatase \text{-} conjugate anti \text{-} rabbit antibody \\ & dilution \text{:} 1 / 7500\end{matrix}$$

After induction at 22° C. over 19 hours, recombinant BLR(DE3) cells expressed the F4 fusion at a very high level ranging from 10-15% of total protein.

In comparison with F4 from the native gene, the F4 recombinant product profile from the codon-optimised gene is slightly simplified. The major F4-related band at 60 kDa, as well as minor bands below, disappeared (see FIG. 10).

### 2.2 Preparation of F4co GMP Lots

A pre-culture was prepared using a frozen seed culture of Escherichia coli strain B 1977. This strain is a BLR(DE3) strain transformed with a pET28b derivative containing a codon-optimized sequence coding for F4 (F4co). The seed culturability was determined as approximately 1E+10 colony forming units per ml.

The seed culture was thawed to room temperature and 500 μl were used to inoculate a 2 litre Erlenmeyer flask (without baffles) containing 400 ml of preculture medium supplemented with 50 μg/ml kanamycin (adapted from Zabriskie et al. (J. Ind. Microbiol. 2:87-95 (1987)), incorporated herein by reference).

The inoculated flask was then incubated at 37° C. (±1° C.) and 200 rpm (New Brunswick Scientific, Innova 4430 with a stroke of 1 inch). The pre-culture was stopped when the optical density at 650 nm (OD_{650 nm}) reached 2, which corresponds to around 5 h30 of incubation. Pre-culture was used for inoculation immediately after it was stopped.

Where inoculation of a large scale fermenter is necessary, multiple batches of pre-culture can be combined.

2.3 Purification of F4co (p24-RT-Nef-p17)—E. Coli Strain B1977

### 2.3.1 Summary

To purify the fusion construct F4co a purification process that comprises two chromatographic steps and a diafiltration for final buffer exchange and protein refolding can be used. The method comprises the following principal steps:
Cell paste homogenization at OD90 and addition of 8M urea
Cation-exchange chromatography on CM hyperZ (positive mode)
Anion-exchange chromatography on QAE 550C (positive mode)
Tangential flow filtration
Sterile filtration

Three reproducibility batches were successfully produced at the final development scale of 1.5 l homogenate OD 90. Consistent results obtained from three different cell-culture harvests demonstrated the robustness of the purification process.

The three lots produced between 1.3 and 1.6 g F4co. Purity of the full-length product was about 93-94% (density scans of Coomassie blue-stained SDS-gel) due to product heterogeneity.

Below is presented the results obtained with the three reproducibility batches.

### 3.3.2 Analytical Methods

The total protein concentration was determined with the Lowry assay.

For SDS-PAGE, samples were prepared in reducing or non-reducing SDS-PAGE sample buffer (+/−β-mercaptoethanol) and heated for 5 min at 95° C.

Proteins were separated on 4-12% SDS-polyacrylamide gels at 150 V for 90 min using either pre-cast NuPage gels or Criterion XT gels (Bio-Rad), 1 mm thick.

Proteins were visualized with Coomassie-blue 8250.

For anti-F4 western blot, the proteins were transferred from unstained SDS-gels onto nitrocellulose membranes (Bio-Rad) at 4° C. for 2 h at 70 V or overnight at 30 V. F4 was detected using anti-F4 antibodies. Alkaline-phosphatase conjugated anti-rabbit antibodies (Promega) were bound to the primary antibodies and protein bands were visualized using BCIP and NBT as the substrates.

For anti-E. coli western blot, proteins were separated by SDS-PAGE and transferred onto nitrocellulose membranes as above. Residual host cell proteins were detected using polyclonal anti-E. coli antibodies. Protein bands were visualized with the alkaline-phosphatase reaction as described above.

Samples were subjected to stability tests at different temperatures (usually −20° C., 4° C., RT, 30° C.) in Eppendorf cups under sterile conditions. Samples were incubated for the indicated times and then analyzed by SDS-PAGE in reducing conditions to detect F4 degradation or in non-reducing conditions to detect aggregates.

For analytical SEC, proteins were separated on an analytical Superdex 200 HR10/30 column (Amersham Biosciences) in 10 mM Tris buffer pH 8.5-0.4M Arginine-10 mM sodium sulfite-1 mM EDTA at a flow rate of 0.5 ml/min. Eluting proteins were on-line monitored at 280 nm.

The LAL test was employed to measure endotoxins in the purified bulk using the kit from Bio Whittaker. E. coli 055:B5 endotoxin was used as the standard. Kinetic curves were recorded at 37° C. in a 96-well spectrophotometer (Spectramax 250, Molecular Devices) and the data were analyzed using SoftmaxPro.

Residual urea was measured with the urea/ammonia test kit from Roche. NADH extinction was monitored with a spectrophotometer (Ultraspec II, Pharmacia) at 340 nm.

### 3.3.3 The Purification Process

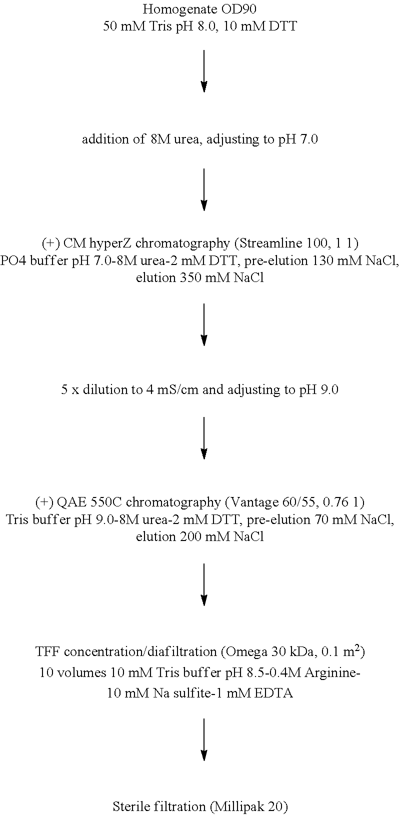

### 2.3.4 Results of the Reproducibility Lots

### Follow-Up

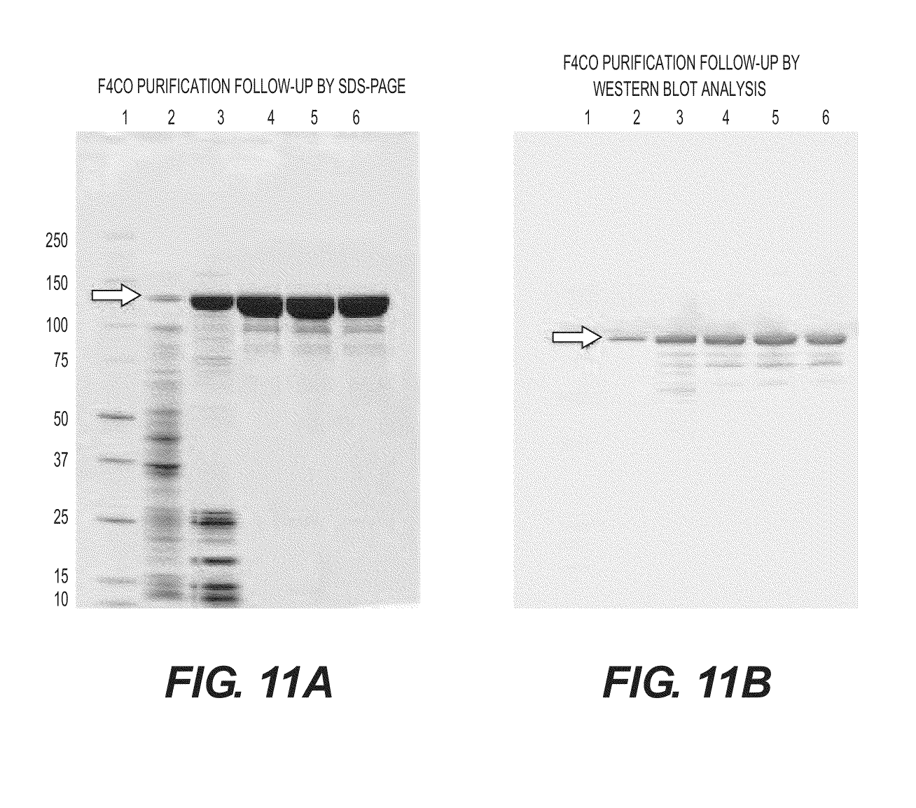

The purification follow-up of lot 1 as a typical example is presented in FIG. 11. Only the F4co positive fractions were analyzed on this SDS-PAGE and the corresponding anti-F4 western blot.

On the gel in FIG. 11A, one can see the increase of the full-length F4co band located at about 130 kDa as the purity of this protein increases. Lane 2 shows the proportion of F4co in the homogenate. F4co was recovered in the CM eluate and many HCP were eliminated (lane 3). Final product purity was already obtained after the second purification step in the QAE eluate (lane 4). F4co was unchanged in the retentate after ultrafiltration (lane 5) and lane 6 represents the purified product. Several low molecular weight bands are visible on the SDS-gel due to product heterogeneity; these bands were also detected on the anti-F4co western blots in FIG. 11B.

### Purity

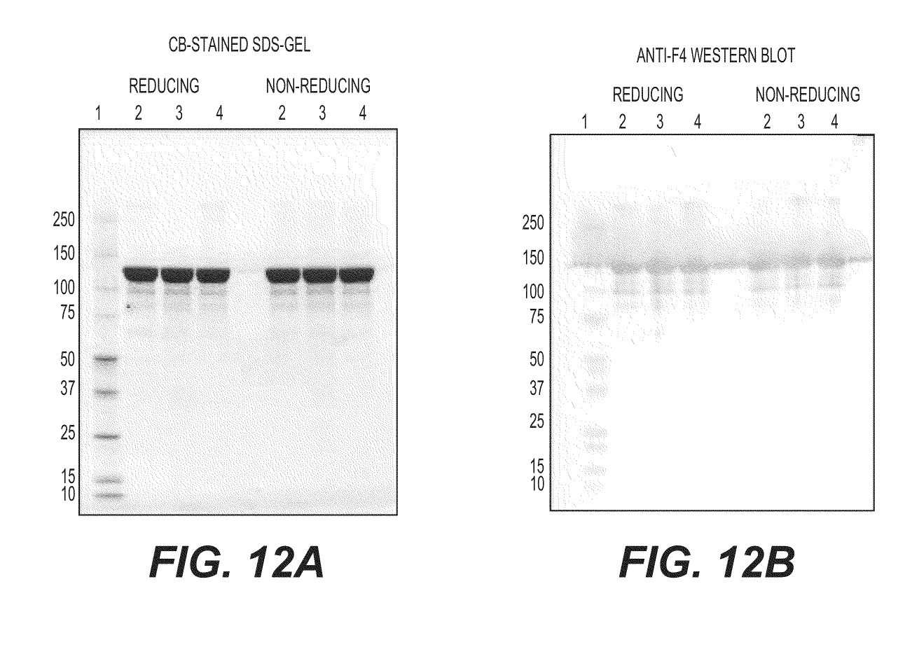

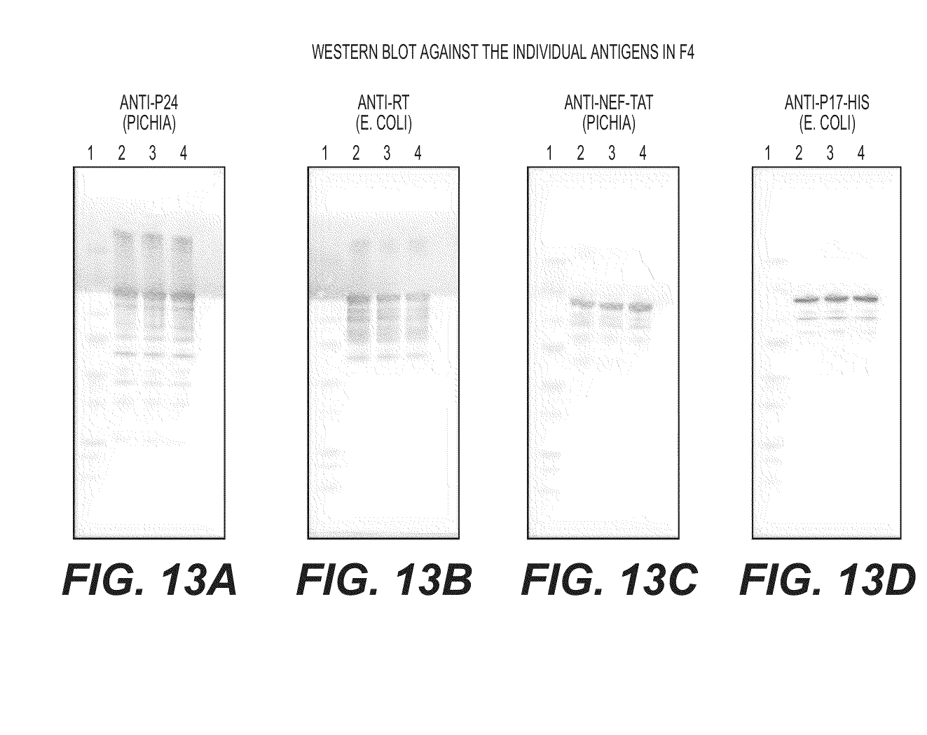

To confirm product consistency, the three purified bulks were compared on the CB-stained SDS-gel and the anti F4 western blot shown in FIGS. 12A and 12B. Additionally, western blots were realized with antibodies directed against the individual proteins (anti-p24, anti-RT, anti-Nef-Tat and anti-p17-His in FIGS. 13A-13D).

### SEC Analysis

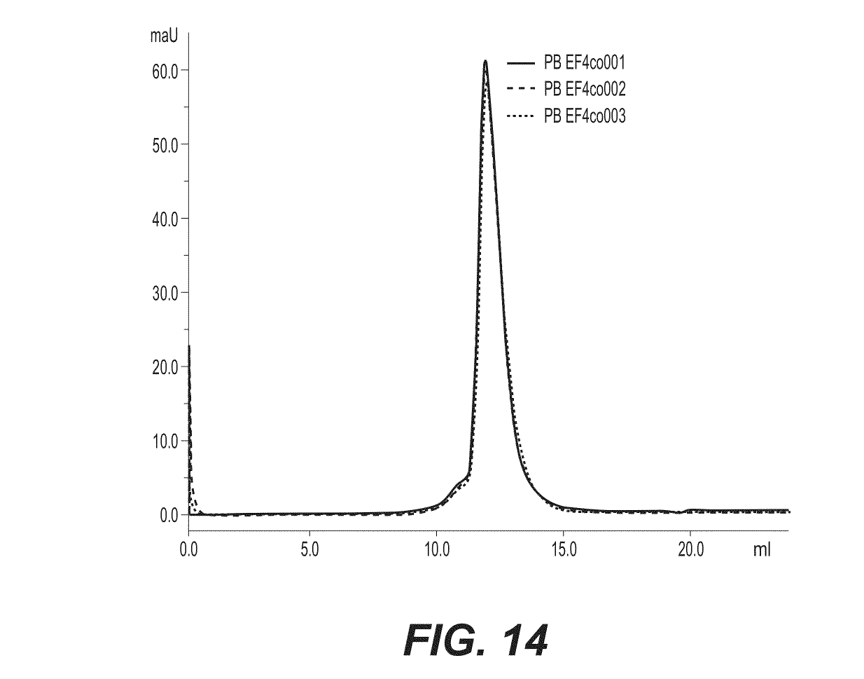

The three purified bulks were additionally compared by size exclusion chromatography SEC analysis on an analytical Superdex 200 column. The three chromatograms are compared in FIG. 14 below.

The similarity of the protein pattern of F4co visible on the SDS-gel in FIG. 12A and on the western blots in FIGS. 12B and 13A-13D, as well as the almost identical elution profiles from the SEC column in FIG. 14, point at the very good lot to lot consistency.

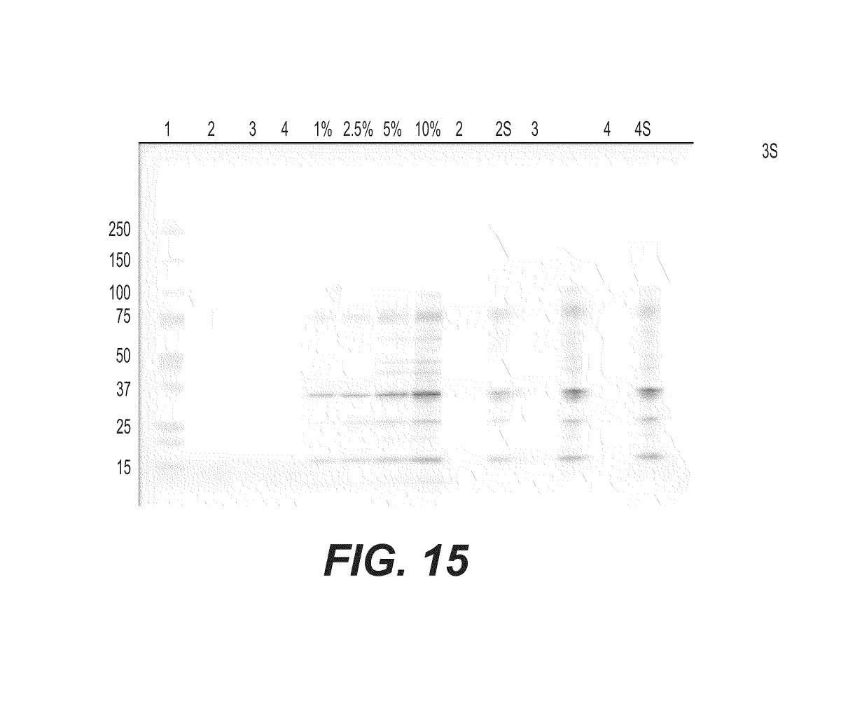

Full-length F4co and the low molecular weight (LMW) bands appear at a similar intensity on the gel and on the western blots. The visible LMW bands on the gel are clearly product related: they are recognized by the anti-F4 antibodies and/or the antibodies directed against the individual proteins and were not detected on the anti-E. coli western blot in FIG. 15 below.

The anti-E. coli western blot was realized with 10 μg protein per lane of each purified bulk. The absence of visible bands is a further indication of the product purity and indicates that the visible bands on the SDS-gel are product related. Comparison with the standard (0.1-1 μg homogenate) indicates that residual HCP contamination was consistently below 1% in all three purified bulks.

Taken together, all these data demonstrate the purity of the product and the absence of impurities but also the heterogeneity of F4co. Importantly, the data demonstrate an excellent lot to lot consistency of the purified material from different fermentation batches.

### Recovery and Yield

F4co recovery after each purification step was estimated on the basis of CB-stained SDS-gels and total protein recovery (protein concentration measured with the Lowry test).

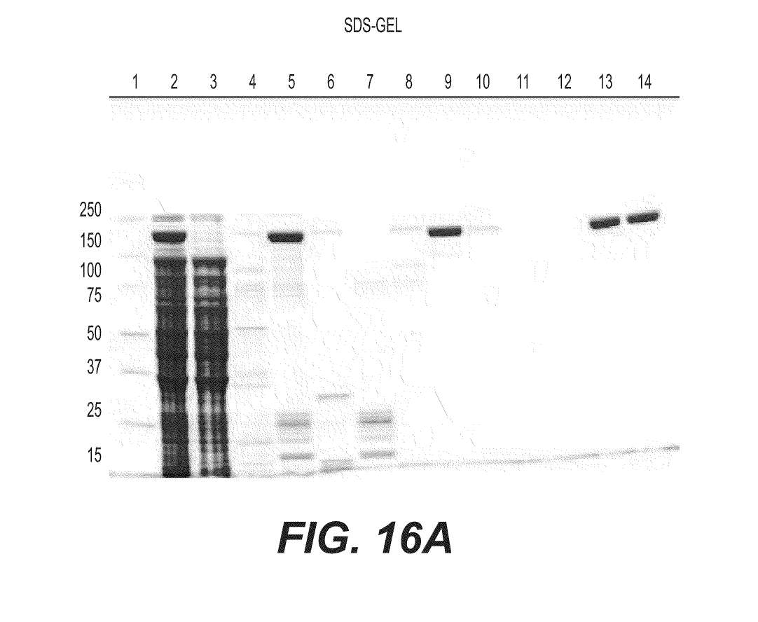

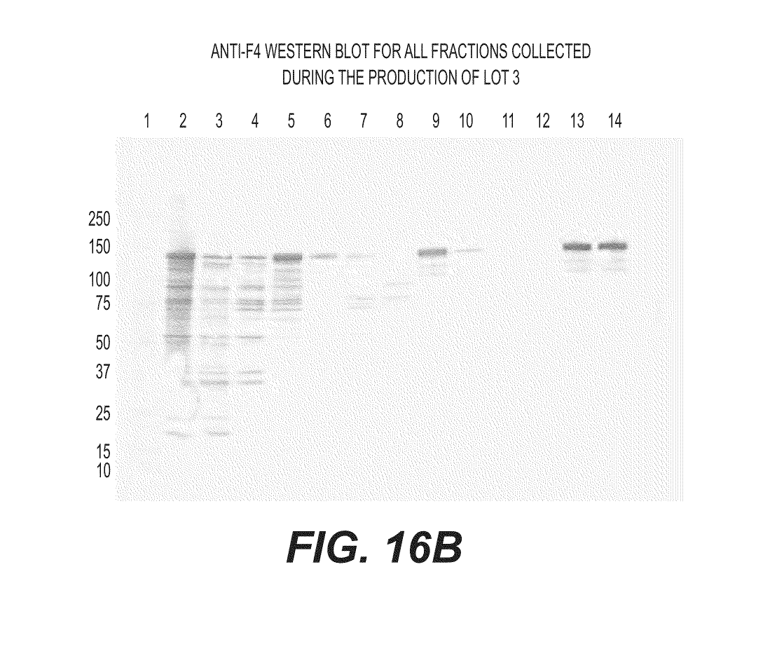

FIG. 16 displays all fractions collected during the production of lot 3 as an example. The sample volumes deposited onto the gel were equivalent to the volumes of each collected fraction and directly related to the homogenate volume in lane 2 of FIG. 16. Visual inspection of the density of the full-length F4co band therefore allows estimation of the recovery.

The SDS-gel shows the homogenate in lane 2 and the initial F4co content. The CM column captured all F4co from the non-clarified homogenate whereas a huge amount of HCP did not interact with the resin at pH 7.0 but was eliminated with the FT (lane 3). A slight loss can be observed in the pre-eluate, resulting in simplification of the product pattern and improved HCP removal. Full-length F4co was nearly quantitatively recovered in the CM eluate with 350 mM NaCl (lane 5).

Almost no loss of F4co was detected in the following steps. Further HCP and LMW F4co-bands removal can be seen in the FT and the pre-eluate of the QAE column (lanes 7 and 8). High F4co recovery and final product purity was obtained in the QAE eluate (lane 9).

Some product loss can be noted after the ultrafiltration step. This loss was estimated at about 10-15% and might be explained by protein adsorption onto the UF membrane because no significant amount of protein was found in the filtrate (lanes 11 and 12).

On the basis of these SDS-gels global F4co recovery appeared higher than 50% in all three lots.

### 3. Immunogenicity of F4 in Human Subjects

### 3.1 Methodology

In the present example, the HIV-1 vaccine candidate contained 10, 30 or 90 μg per dose of F4 recombinant protein as active ingredient, adjuvanted with AS01_{B} or reconstituted with water for injection (WFI).

The vaccine antigen was prepared as a lyophylized pellet containing the F4 antigen in sucrose, EDTA, arginine, polysorbate 80 and sodium sulfite in phosphate buffer. The AS01_{B} liposome-based adjuvant system contains 50 μg MPL and 50 μg QS21 and was prepared in accordance with Example 1 above.

The freeze-dried fraction containing the F4 antigen and the liquid fraction containing the AS01_{B} adjuvant system or WFI, both presented in a single-dose 3 ml glass vial, were reconstituted by the person administering the vaccine shortly before injection. After dissolution of the vial contents, 0.5 ml of the reconstituted vaccine solution was withdrawn into a syringe for intramuscular administration.

Participants were healthy male and female adults aged 18-40 years at low risk of HIV-1 infection, who had not previously participated in another HIV-1 vaccine study or received MPL. Subjects were required to be seronegative for antibodies against the core antigen of HBV (HBc), hepatitis C virus (HCV), HIV-1 and HIV-2, and negative for HBV surface antigen (HBs Ag) and HIV-1 p24 antigen on screening sera within the 8 weeks prior to first vaccination (all tests Abbott AxSYM). Standard eligibility criteria were used for enrolment into the study, as detailed in the ClinicalTrials.gov registry.

One-hundred and eighty subjects were randomized 5:1 between adjuvanted and non-adjuvanted groups. Three groups of 50 subjects received escalating doses of 10, 30 or 90 μg F4 in AS01B and three groups of 10 subjects received escalating doses of F4 in water for injection (WFI). The study was observer-blind for adjuvantation, but open for antigen content. Each subject received a first vaccine dose at Month 0 and a second at Month 1, by injection into the deltoid muscle of the non-dominant arm. Blood samples were obtained prior to vaccine administration (Day 0), two weeks (Day 44) and one month (Day 60) following the second vaccine dose and at Months 6 (Day 180) and 12 (Day 360) for evaluation of safety and immune responses.

The mean (±SD) age of study participants was 22.3 (±4.62) years (range: 18-40 years), 63.3% were female and the population was predominantly white/Caucasian (96.7%). No differences in baseline demographics were observed between the six study groups. All subjects received two doses of the study vaccine and 176 of 180 subjects completed the study. One-hundred fifty subjects (83.3%) were included in the protocol immunogenicity cohort for Month 12. Reasons for exclusion from the analysis are summarized below.

### 3.2 Safety

Solicited local (injection site pain, redness, swelling), general (fever, fatigue, headache, sweating, myalgia) and gastrointestinal (nausea, vomiting, diarrhea, abdominal pain) symptoms were recorded on diary cards for seven days after each vaccine dose. The symptom severity was graded on a scale of 1 to 3, with grade 3 symptoms defined as redness or swelling of more than 50 mm in diameter, or fever above 39.0° C. and for any other symptom as preventing normal daily activities. Unsolicited symptoms were recorded for thirty days after each vaccine dose, whereas serious adverse events (SAEs) were recorded throughout the study.

The analysis of reactogenicity and safety was performed on the total vaccinated cohort. The number and percentage of subjects reporting solicited and/or unsolicited local and general symptoms were calculated with exact 95% confidence interval (CI). No formal statistical comparison of safety data was performed.

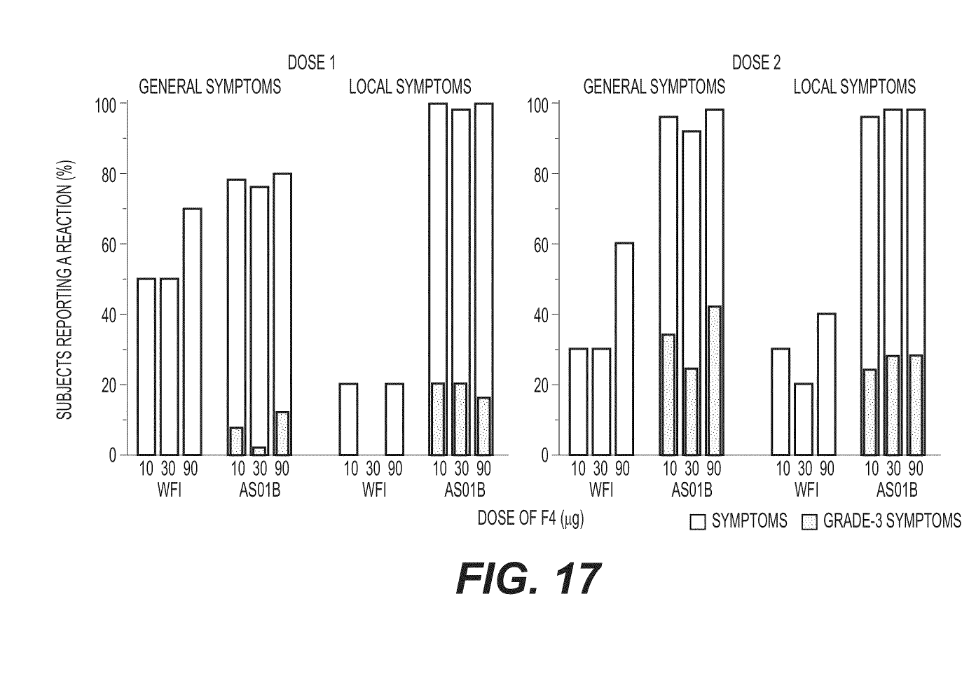

Following each vaccine dose, a markedly higher reactogenicity was observed during the seven-day post-vaccination period in the F4/AS01_{B} groups as compared to the F4/WFI groups (FIG. 17). The overall incidence of solicited and unsolicited symptoms during the seven-day post-vaccination period was 99.0-100.0% of doses in the F4/AS01_{B} groups compared with 45.0-75.0% in the F4/WFI groups. Furthermore, the incidence of general symptoms was higher in the F4/AS01_{B} groups after the second vaccine dose. In the F4/AS01_{B} groups, approximately one-third of all doses were followed by grade 3 symptoms. In contrast, no solicited or unsolicited grade 3 symptoms, related to vaccination, were reported in the F4/WFI groups.

No differences in reactogenicity were observed between the antigen dose levels in the F4/AS01_{B} groups. Pain at the injection site was the most common solicited local symptom. It occurred following 96.0-98.0% of doses in the F4/AS01_{B} groups as compared to 10.0-30.0% of doses in the F4/WFI groups. Pain at the injection site of grade 3 severity occurred after ≦10.1% of doses in the F4/AS01_{B} groups. Redness and swelling were only reported in the F4/AS01_{B} groups, and occurred after 19.0-35.0% and 20.0-25.0% of doses, respectively. The most common general solicited symptoms were fatigue (66-77%, 30-45% of dosed) and headache (58-62%, 20-25% of doses) in the F4/AS01_{B} and F4/WFI groups respectively. The overall per-dose incidence of a given grade 3 solicited general symptom was ≦12.1% in the F4/AS01_{B} groups.

During the 30-day post-vaccination period, 50.0-70.0% of subjects in the F4/WFI groups reported unsolicited symptoms, compared to 60.0-84.0% of subjects in the F4/AS01_{B} groups. Symptoms were considered causally related to vaccination in 30.0-44.0% of subjects in the F4/AS01_{B} groups (most frequently chills and injection site reactions) and only few were of grade 3 severity. Symptoms were, on the whole, transient and resolved without sequelae, generally within two to three days. Throughout the study period, six SAEs were reported in the F4/AS01_{B} groups. All were considered unrelated to vaccination and resolved without sequelae. No deaths occurred during the study period and no subject withdrew from the study due to adverse events.

### 3.3 T-Cell Responses

The CD4+ T-cell responses were evaluated by intracellular cytokine staining (ICS) following stimulation with p17, p24, RT and Nef peptide pools to assess the expression of interleukin 2 (IL-2), interferon gamma (IFN-γ), tumor necrosis factor alpha (TNF-α) and CD40-ligand (CD40L). The ICS carried out was an adaptation of previously described methodology [see Maecker H T, Maino V C, Picker L J (2000) Immunofluorescence analysis of T-cell responses in health and disease. J Clin Immunol 20: 391-399 and Maecker H T, Dunn H S, Suni M A, Khatamzas E, Pitcher C J, et al. (2001) Use of overlapping peptide mixtures as antigens for cytokine flow cytometry. J Immunol Methods 255: 27-40, each incorporated herein by reference] using peripheral blood mononuclear cells (PBMC) that were isolated from whole blood cells by standard Ficoll-Isopaque density gradient centrifugation within six hours following blood sampling, and cryopreserved in liquid nitrogen until further analysis.

In brief, the thawed PBMC were stimulated in vitro with pools of 15 mer peptides overlapping by 11 amino acids (Eurogentec, Belgium) covering the sequences of clade B p17, p24, RT or Nef matched antigens, in the presence of anti-CD28 and anti-CD49d antibodies (BD Biosciences, Belgium). After two hours of incubation at 37° C., the intracellular blocking agent Brefeldin A (BD Biosciences, Belgium) was added to inhibit cytokine secretion during an additional overnight incubation. Cells were subsequently harvested, stained for surface markers CD4+ and CD8+ (BD Biosciences), and then fixed (Cytofix/Cytoperm kit, BD Biosciences). The fixed cells were then permeabilized and stained with labeled antibodies to IL-2, IFN-γ, TNF-αc and CD40L (BD Biosciences), washed, resuspended in foetal-calf-serum-containing phosphate buffered saline and analysed by flow cytometry using a FACSCanto flow cytometer and FACSDiva software (BD Biosciences) or FlowJo software (Tree Star).

In order to evaluate the cross-clade reactivity of the vaccine-induced CD4+ T-cells, PBMC collected at Days 0 and 44 were analyzed by ICS for the expression of CD40L and production of IL-2, IFN-γ and TNF-αc using peptide pools from consensus sequences of clade A, and C HIV-1 strains. The clade A sequences used for p24 and p17 come from native isolate TZA173 (Tanzania) and the clade A sequences used for RT and Nef come from native isolate KE MSA4070 (Kenya). Clade C sequences for all antigens were from strain ZM651. This exploratory survey was performed as described above but only on samples from subjects from the F4 10 μg/AS01_{B} group.

The ICS results were expressed as the frequency (in percent) of the total CD4+ and CD8+ T-cells, respectively, expressing the immune markers IL-2, IFN-γ, TNF-αc and/or CD40L, in response to stimulation with p17, p24, RT or Nef antigens. A subject was considered a responder if the antigen-specific CD4+ response was greater than or equal to the cut-off value. A cut-off value of 0.03% double-positive antigen-specific CD4+ T-cells (i.e. cells expressing at least two markers from IL-2, IFN-γ, TNF-αc and CD40L) was selected on the basis of the maximum value (rounded to the superior hundred) among all 95th percentiles of the double positive antigen-specific CD4+ T-cell, for the different antigens.

The analysis of immunogenicity was performed on the according-to-protocol immunogenicity (ATP) cohort. The frequency of CD4+ T-cells expressing IL-2 and at least one other marker and the percentage of responders following in vitro stimulation by each individual antigen and by at least 1, 2, 3 and all 4 antigens was determined at each time point. The F4-specific CD4+ T-cell response was defined as the sum of the specific CD4+ T-cell frequencies in response to each individual antigen.

A two-way ANOVA statistical test was performed on the frequency of CD4+ T-cells expressing IL-2 and at least one other marker to compare three doses of F4 with or without adjuvant 2 weeks after the second vaccine dose. The ANOVA model included the doses (10, 30 and 90 μg) and the adjuvants (AS01_{B} and WFI) as fixed effects. To achieve a normally distributed response, the analysis was performed on the log 10 frequency of CD4+ T-cells. The criteria of equality of variances were not fulfilled between adjuvanted and non-adjuvanted groups.

As the interaction between doses and adjuvant was significant (p≦0.05) for the in vitro stimulation by most of the antigens (except p17) and because the difference between AS01_{B} and WFI was clearly high, a one-way ANOVA was performed on the log 10 frequency of CD4+ T-cells expressing IL-2 and at least one other marker to compare the three doses of F4 with AS01_{B} 2 weeks post-dose II (Day 44). The one-way ANOVA model included the doses (10, 30 and 90 μg) as a fixed effect. The criteria of equality of variances were fulfilled between the three AS01_{B} groups. ANOVA analyses were carried out for the CD4+ T-cell responses to the F4 antigen and each of its components.

Multiple comparisons (Tukey-Kramer adjustment) were performed and the relationship between doses and CD4+ T-cell responses were also assessed. Geometric mean (GM) ratios and their CIs were tabulated. Analyses were done for the CD4+ T-cell responses to the F4 antigen and each of its components.

### 3.4 CD4+ T-Cell Responses Against the Homologous Antigens

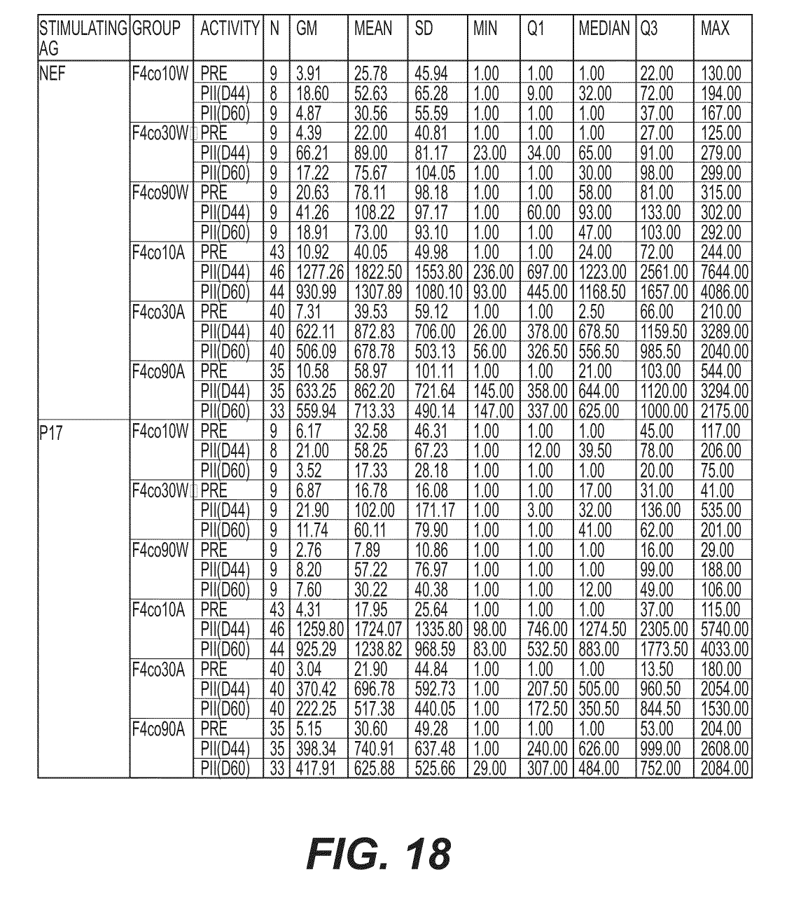

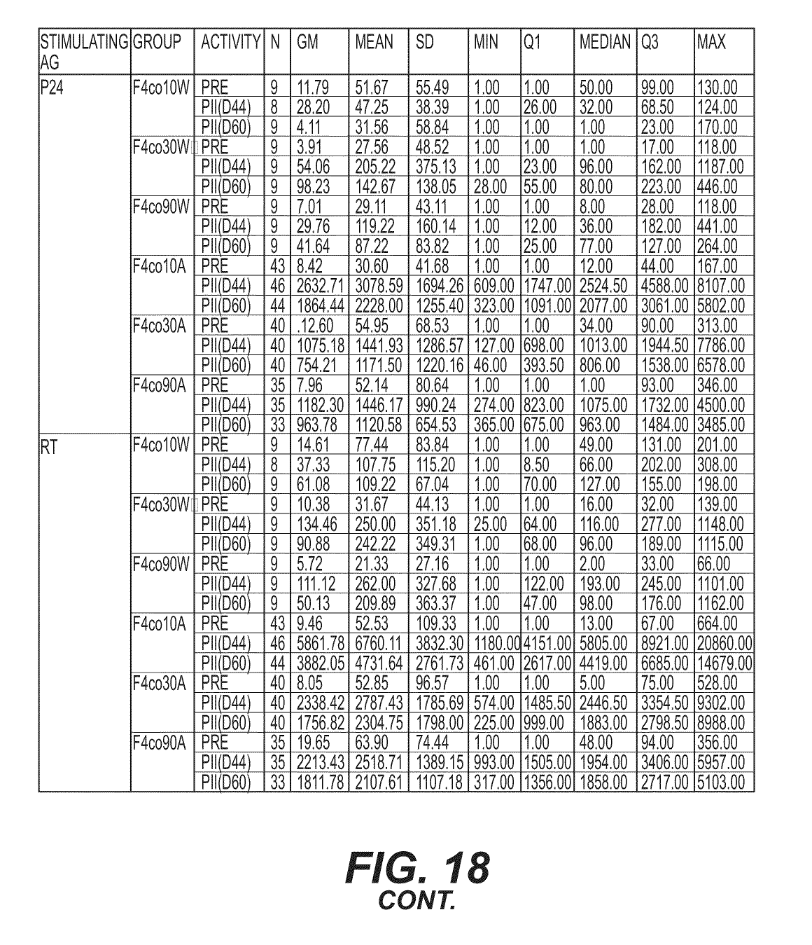

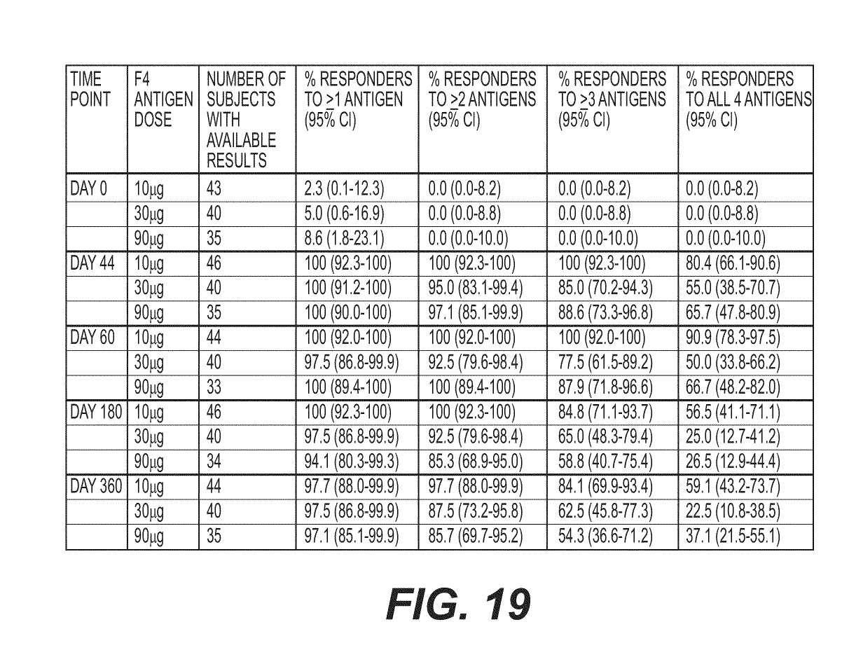

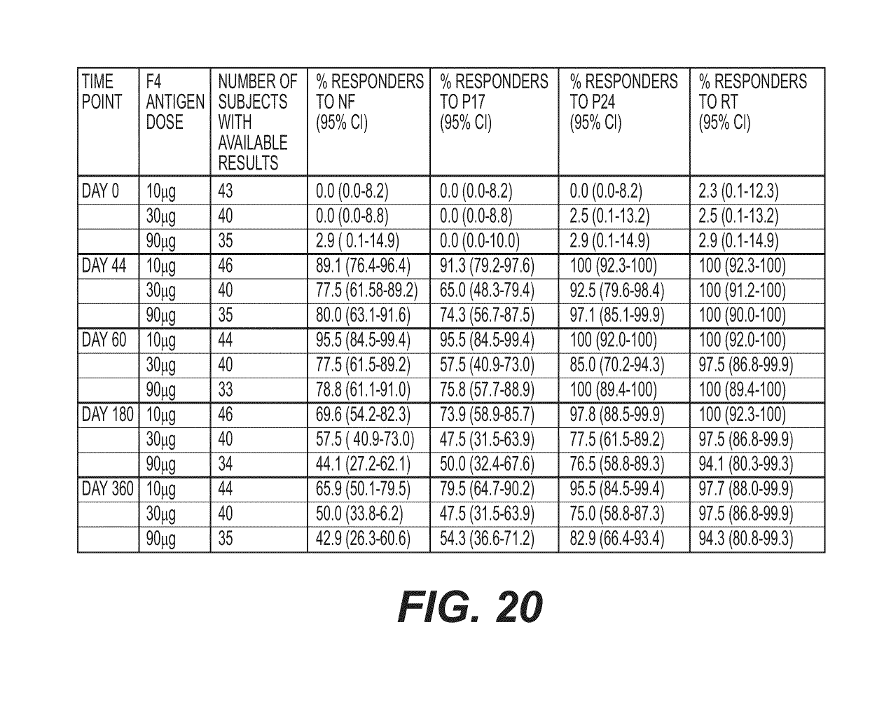

In all non-adjuvanted vaccine groups, the frequency of antigen-specific CD4+ T-cells expressing at least two immune markers including IL-2 was in most cases below or close to the cut-off value (see FIG. 18). In contrast, very high responder rates were observed in all F4/AS01_{B} groups (FIG. 19). The highest percentage of responders was seen in the 10 mg F4/AS01_{B} group, two weeks after the second vaccine dose (Day 44), with all vaccinated subjects responding to at least three antigens and 80.4% to all four antigens. When examining the CD4+ T-cell responder rates per antigen (FIG. 20), it becomes clear that the responses in the F4/AS01_{B} group were very broad and directed against all four vaccine antigens, but were of higher frequency following stimulation with the RT antigen.

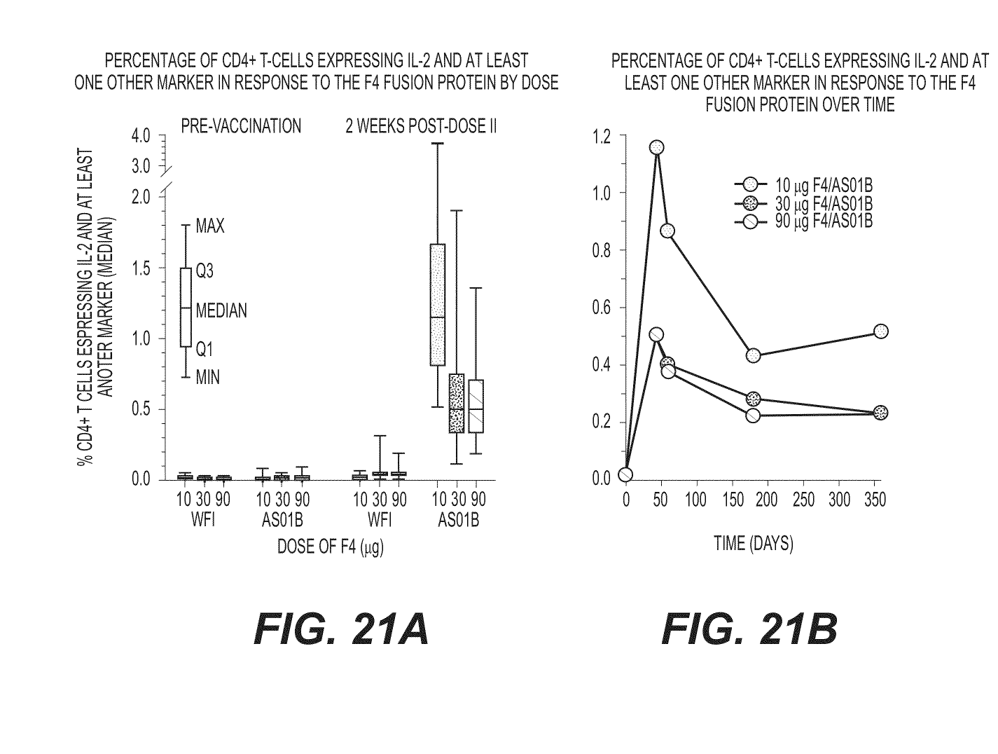

The vaccine-induced CD4+ T-cell responses were long-lived, since 97.7% of subjects in the 10 mg F4/AS01_{B} group were still responding to two antigens at Day 360 and 84.1% and 59.1% to three and four antigens, respectively (FIG. 19). The overall response to the F4 fusion protein was greater and more persistent in the F4 10 μg/AS01_{B} group (p<0.0001 at Day 44) (FIG. 21). In this group, the geometric mean frequency of F4-specific CD4+ T-cells producing IL-2 and at least one other marker peaked at almost 1.2% on Day 44.

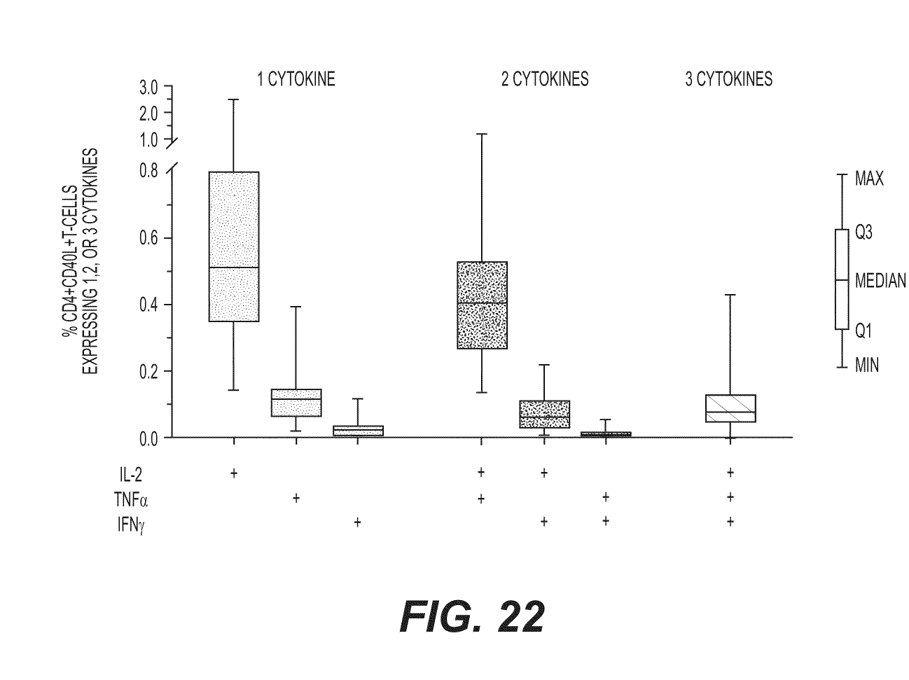

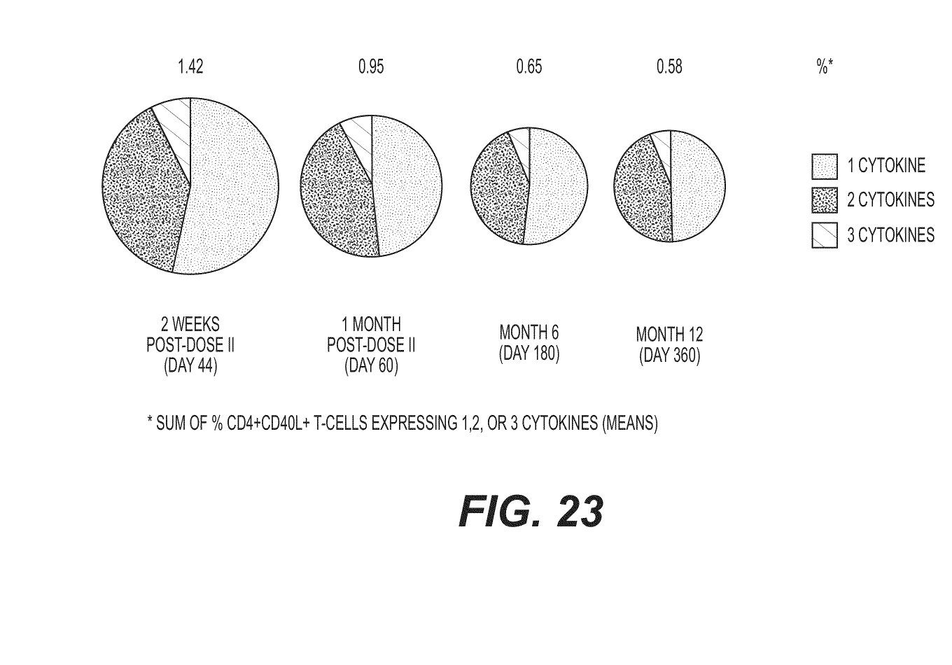

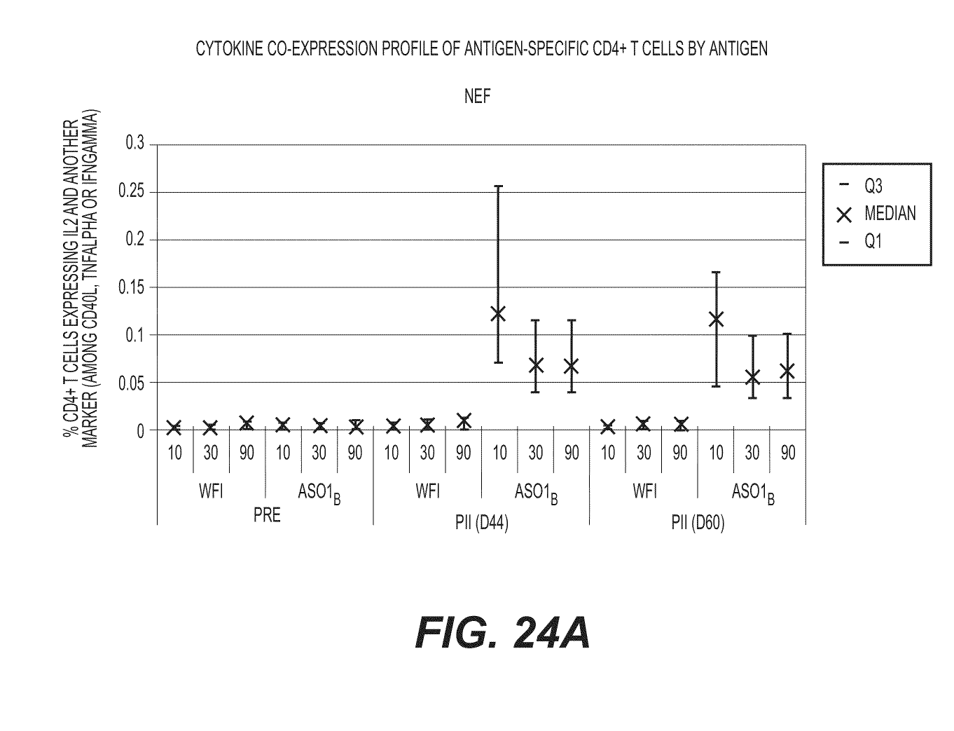

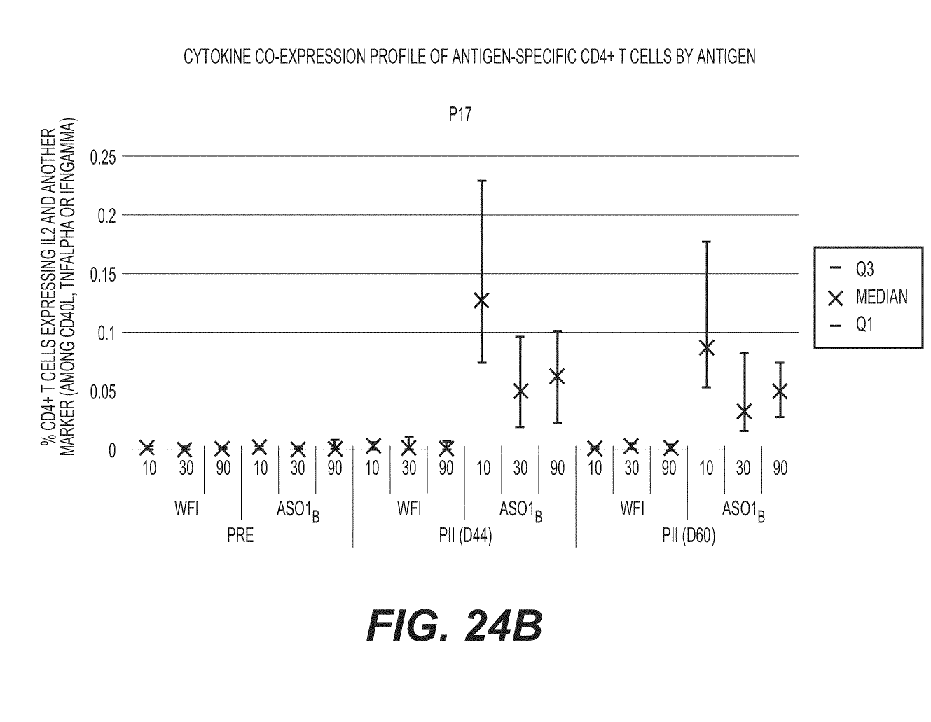

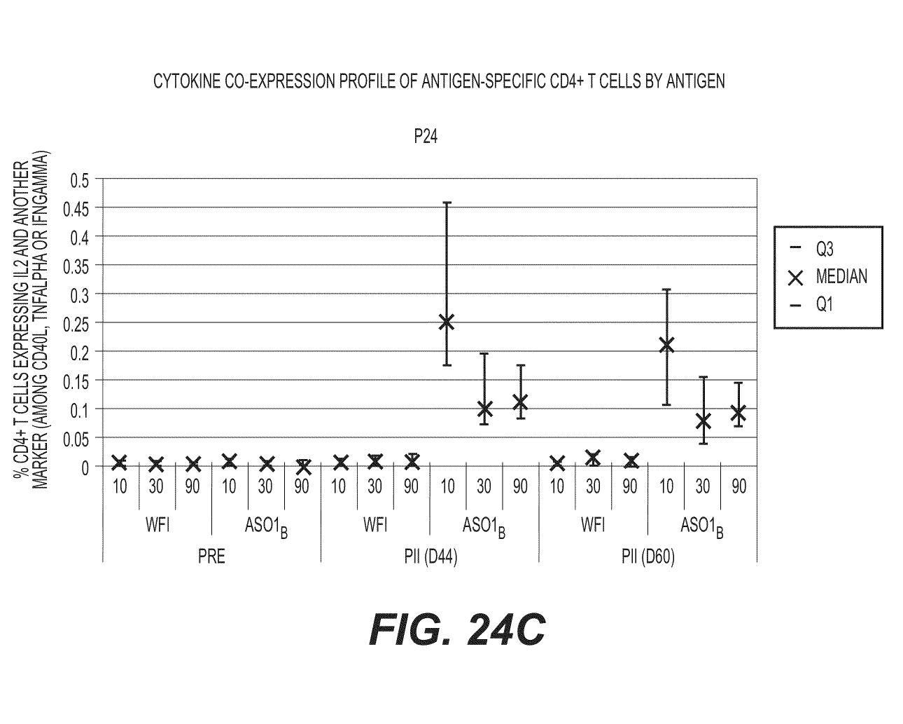

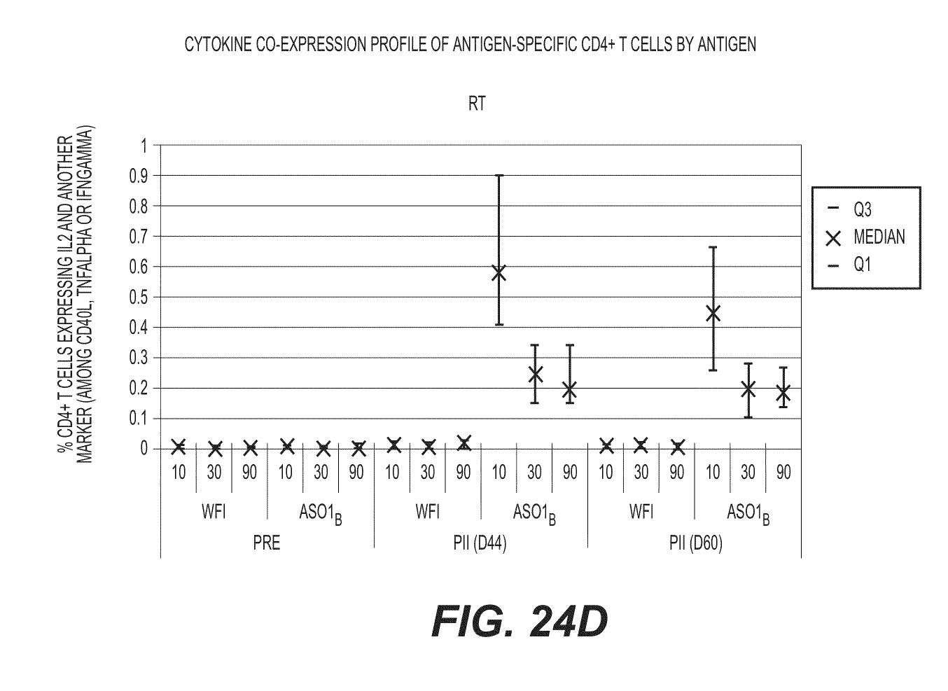

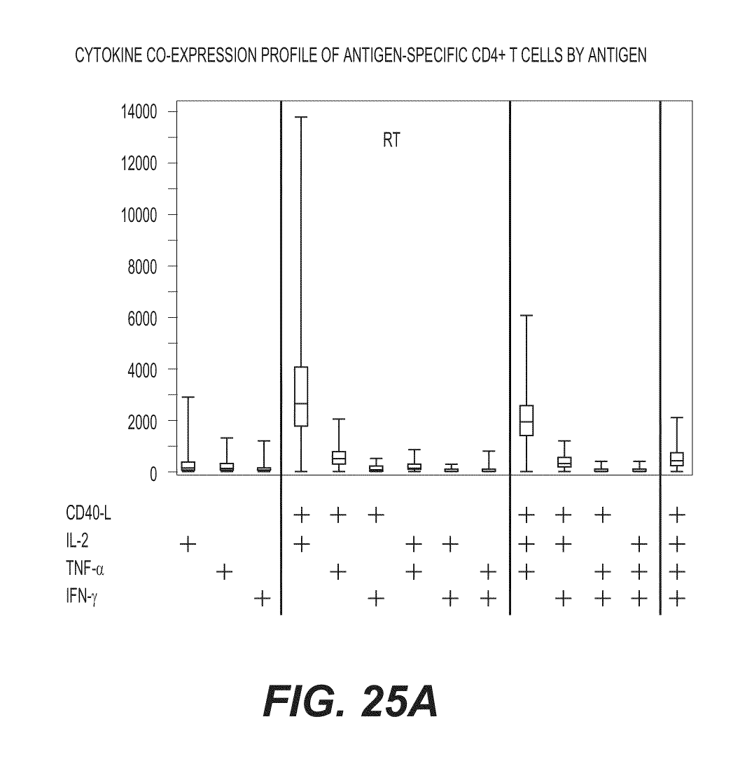

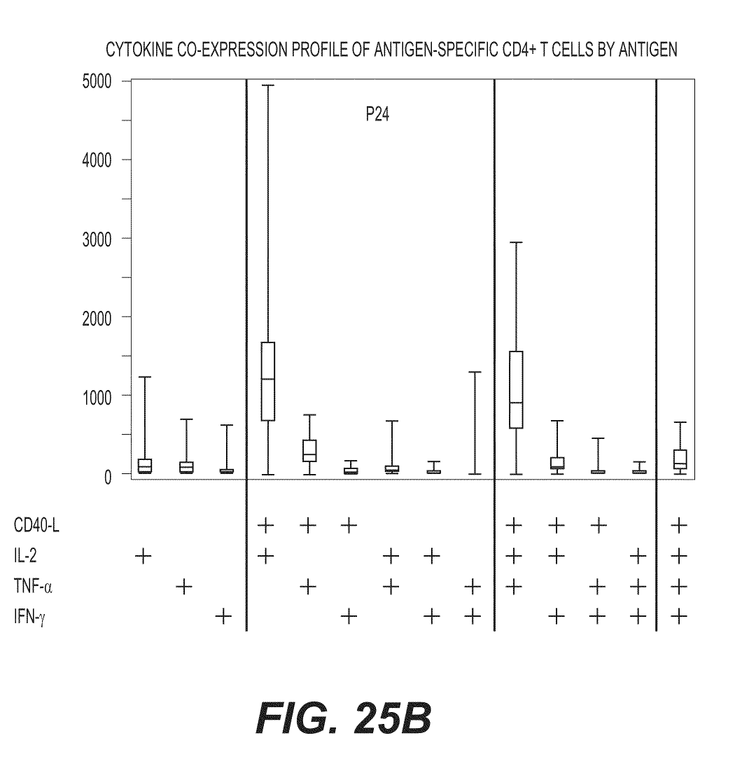

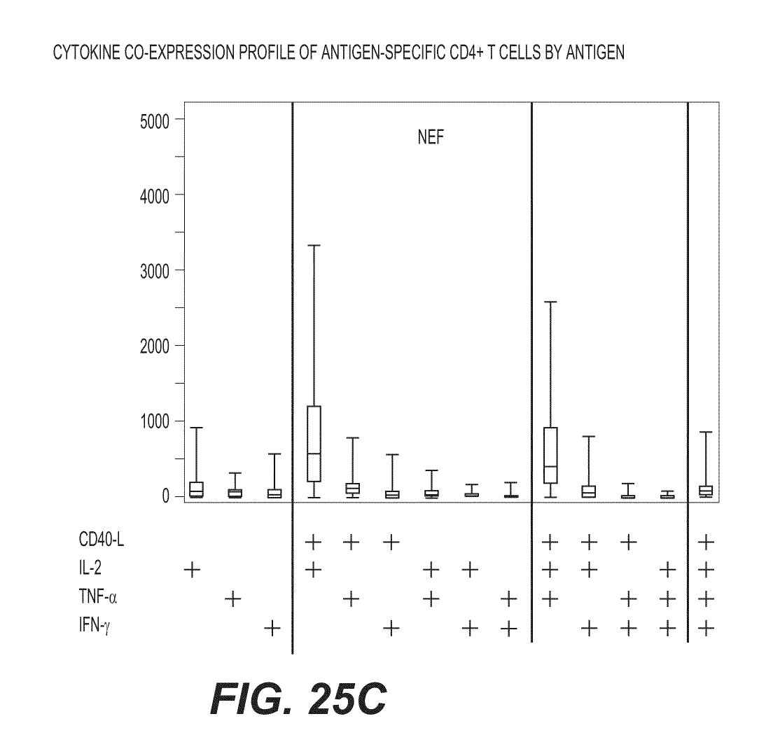

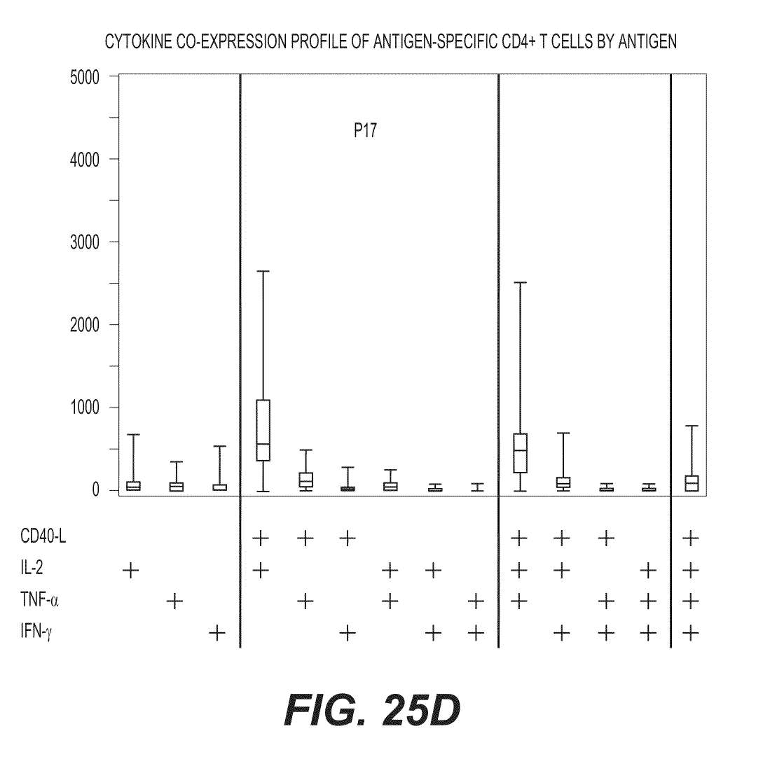

The F4/AS01_{B} vaccine induced polyfunctional F4-specific CD4+ T-cells, as demonstrated by their cytokine co-expression profiles in the 10 μg F4/AS01_{B} group (FIG. 22). The majority of specific CD4+ T-cells expressed CD40L and produced IL-2 alone or in combination with TNF-αc and/or IFN-γ. Approximately 50% of F4-specific CD4+ T-cells secreted at least two cytokines and this cytokine co-expression profile was maintained up to Month 12 (FIG. 23). A similar profile was observed for all individual antigens (see FIGS. 24 & 25).

### 3.5 CD8+ T Cell Responses

Based on the ICS method, vaccine-induced CD8+ T cells have not, as yet, been detected.

### 3.6 CD4+ T-Cell Responses Against Heterologous Antigens (Cross-Clade Reactivity)

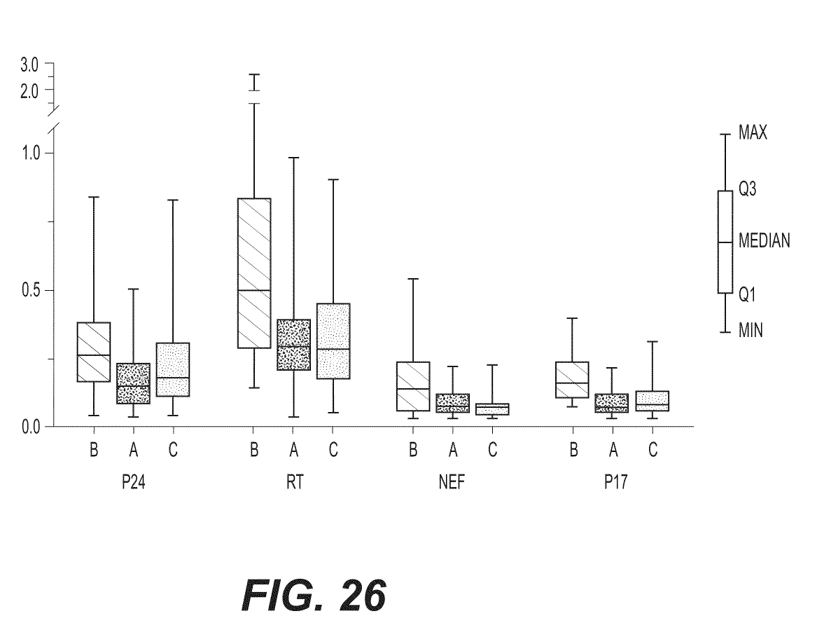

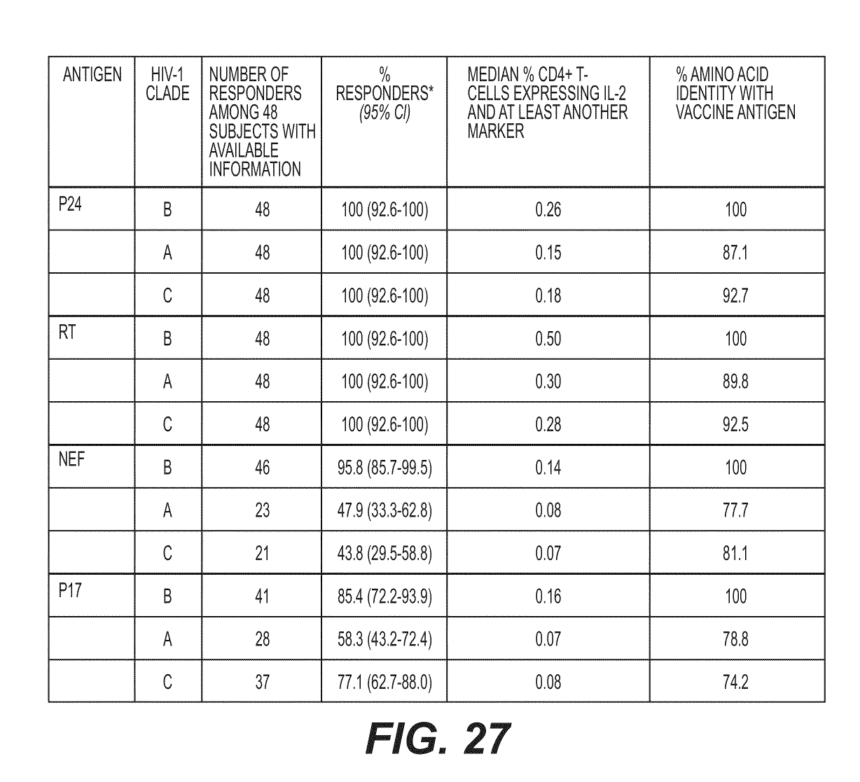

In order to assess the cross-reactivity with HIV-1 non-clade B antigens of the vaccine-induced CD4+ T-cell response in the 10 μg F4/AS01_{B} group at Day 44, responses were analyzed following in vitro stimulation with antigens from clade A and C, together with the clade B antigens that were included as a control. ICS and flow cytometry analysis using p17, p24, RT and Nef peptide pools revealed broadly cross-reactive CD4+ T-cell responses to all four antigens of clade A and C (FIG. 26). Interestingly, every volunteer having received the 10 μg F4co dose had mounted a response to RT and p24 from the homologous clade B and also exhibited a response to both clade A and C corresponding antigens (FIG. 27).

It is surprising that such high levels of cross-reactivity are observed when compared to the relatively low level of epitopic conservation in these antigens across different clades.

### 3.7 Humoral Immune Response

The presence or absence in serum of an immunoglobulin G (IgG) antibody response to p17, p24, RT, Nef and F4 was analyzed using enzyme-linked immunosorbent assays (ELISA). A positive control and calibrators were run on each plate in order to assess the relative concentration of each test sample, as well as negative controls to ensure specificity. The plates were read at 450 nm on a VersaMax plate reader (Molecular Devices, Berkshire, United Kingdom) and analyzed using SoftMax Pro 3.1.1. software (Molecular Devices). Seropositivity was defined as an antibody concentration greater than or equal to the assay cut-off value (≧187 mEU/ml for p17, ≧119 mEU/ml for p24, ≧125 mEU/ml for RT, ≧232 mEU/ml for Nef and ≧42 mEU/ml for F4). The cut-off value was chosen on the basis of the pre-vaccination responses for all subjects. Selection was made on the basis of 95% percentiles at pre-vaccination for Nef, and on the basis of the 99% percentiles at pre-vaccination for F4co, p17, p24 and RT.

Seropositivity rates and geometric mean antibody concentrations (GMCs) for each individual antigen and the fusion protein were calculated with 95% CIs. For seropositivity rates, 95% CIs were computed using the exact method for binomial variables. The 95% CIs for GMCs were calculated by taking the anti-log of the 95% CI of the mean log 10-transformed antibody concentrations. Antibody concentrations below the cut-off of the assay were given an arbitrary value of half the cut-off for the purpose of GMC calculation.

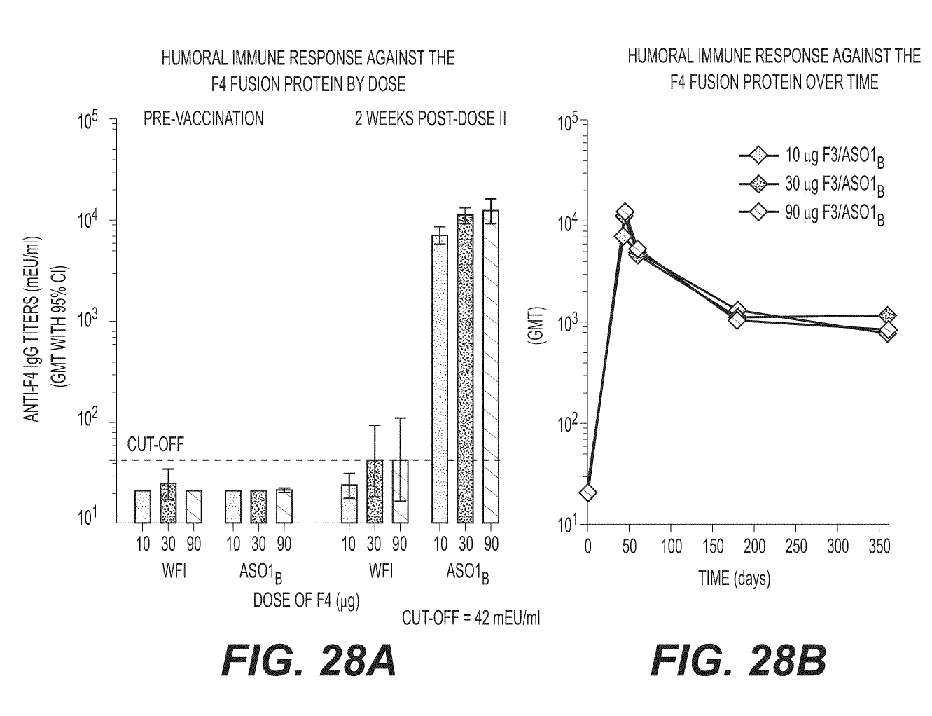

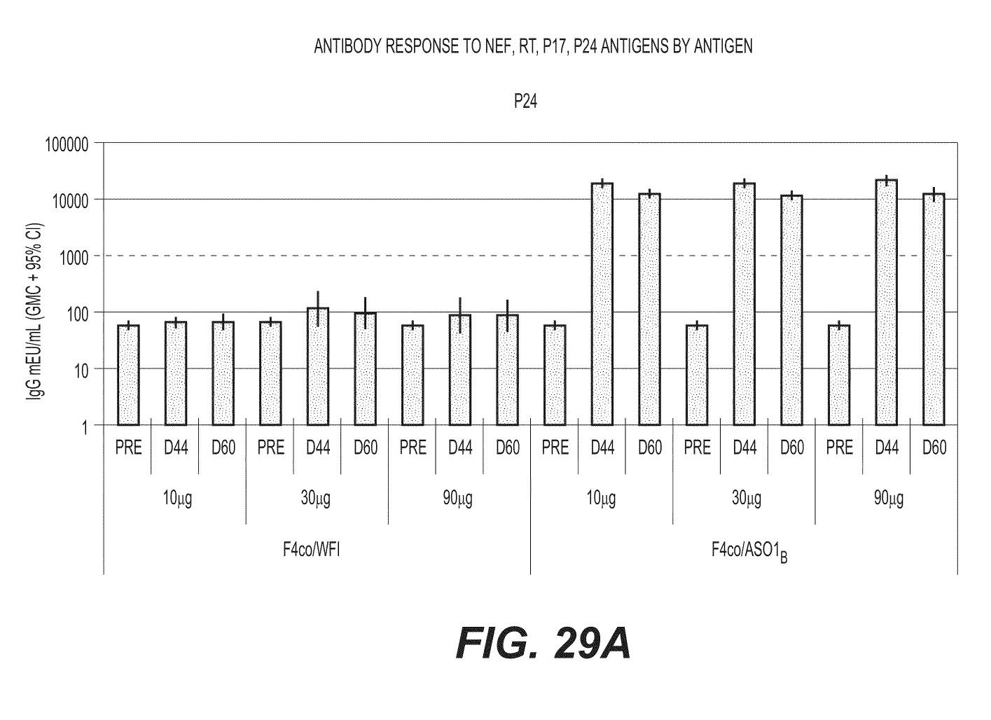

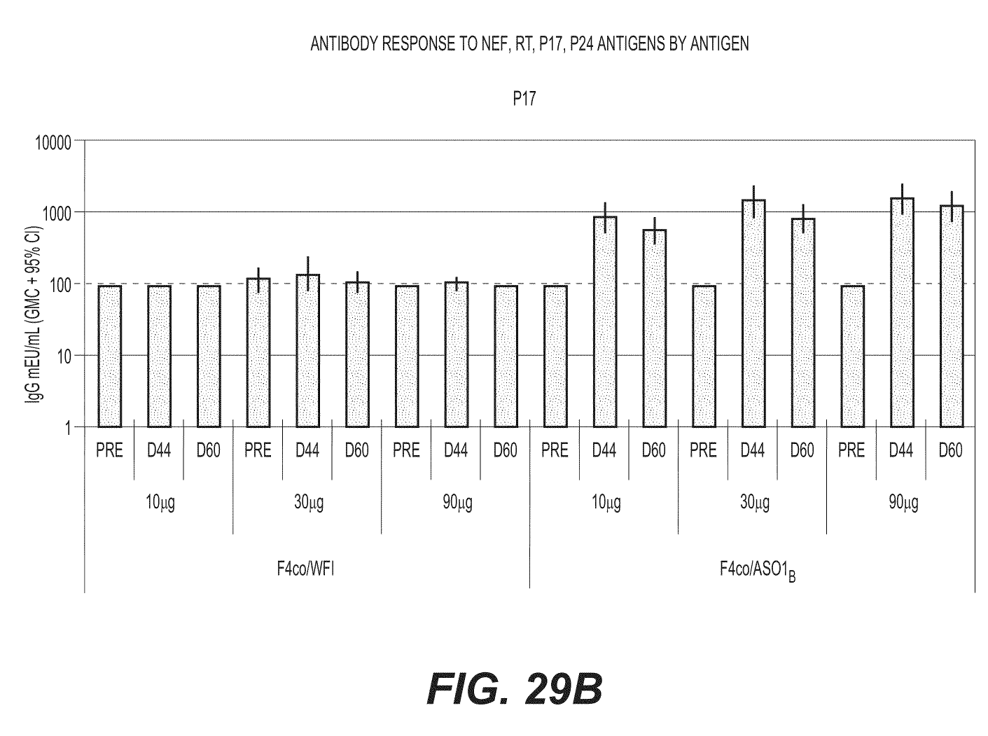

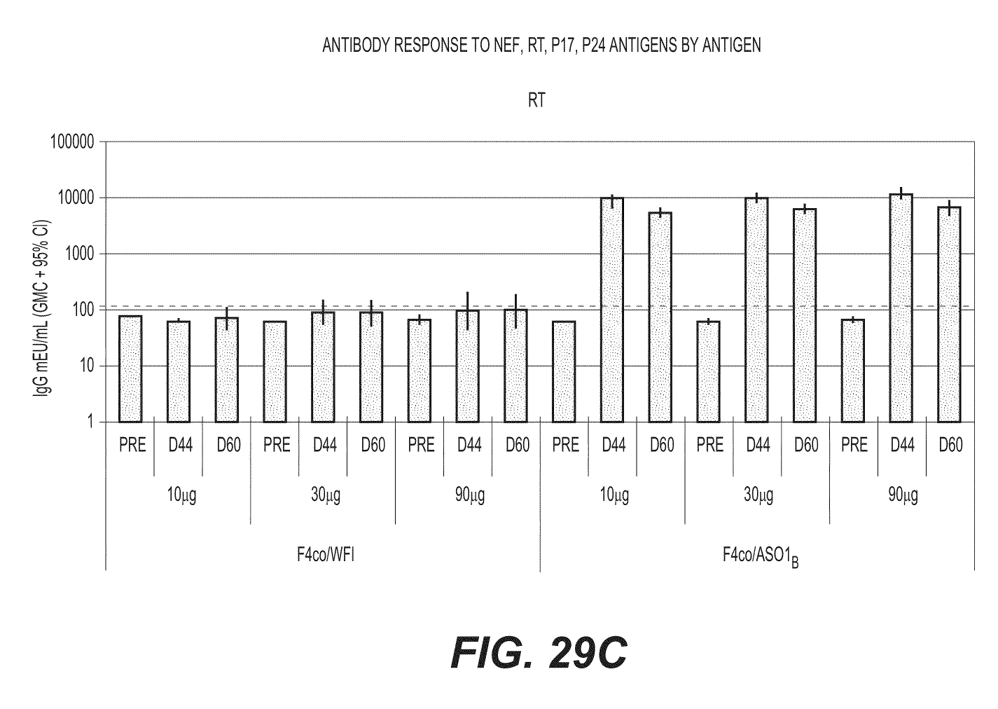

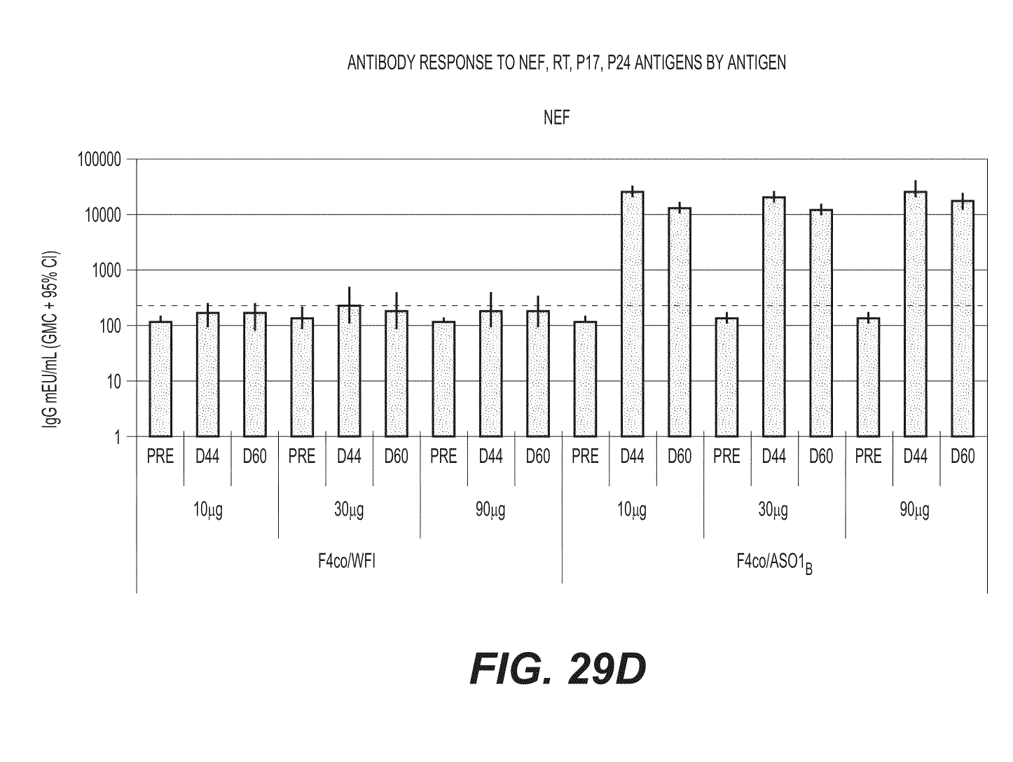

Humoral immune responses were characterized by strong antibody concentrations against the F4 fusion protein in the AS01_{B} groups (FIG. 28). A 100% seroconversion rate to F4 was observed in all adjuvanted groups, with similar IgG titers for all dose levels that persisted up to Month 12. Furthermore, IgG antibodies were elicited against each individual F4 antigen component. In the non-adjuvanted groups, very low responses were induced (see FIG. 29).

### 3.8 Conclusions

The results show that the reactogenicity profile of the F4/AS01_{B}-adjuvanted HIV-1 vaccine was acceptable, without any safety concerns. In addition, the immunogenicity results indicate that the F4/AS01_{B}-adjuvanted HIV-1 vaccine candidate elicited high and long-lasting numbers of HIV-1-specific polyfunctional CD4+ T-cells. Particularly prominent was the overall high rate of responders in the adjuvanted vaccine groups, with responses elicited against all vaccine antigens. Interestingly, the highest responder rates were observed in the lowest antigen dose group (10 μg F4/AS01), with 100% of participants responding to three HIV-1 antigens and 80% to all four HIV-1 antigens. The adjuvanted vaccine groups were characterized by a very high frequency of F4-specific CD4+ T-cells that persisted up to Month 12.

The finding that the vaccine-induced CD4+ T-cells expressed CD40L and produced IL-2 alone or in combination with TNF-αc and/or IFN-γ is an important and promising observation, since multiple-cytokine-producing antiviral CD4+ T-cells are considered to be functionally superior to single-cytokine-producing cells.

Furthermore, the results of this study show that the F4/AS01_{B}-adjuvanted HIV-1 vaccine, comprised of clade B antigens only, was able to elicit broadly cross-reactive CD4+ T-cell responses to all four antigens from clades A and C as well. The induction of a broadly cross-reactive and long-lasting immune response is an important consideration in the development of an HIV-1 vaccine, given the diversity of the HIV-1 virus worldwide.

In conclusion, the results of this study demonstrate the safety and immunogenicity of the F4/AS01_{B}-adjuvanted HIV-1 vaccine candidate. Strong polyfunctional, broadly reactive and persistent CD4+ T-cell responses were induced with two vaccine doses containing 10 μg of the F4 protein adjuvanted with AS01. The properties of this immune response make this vaccine a promising AIDS vaccine candidate, not only in a prophylactic setting but also as a disease-modifying therapeutic vaccine.

4. Immunogenicity of F4Co Derived from Different Clades

In the present example, T cell responses elicited by codon-optimised F4 derived from clades B and C were tested for cross-reactivity against peptides from clades A, B and C.

### 4.1 Methodology

The clade B F4co protein used for immunisation was prepared as described in Example 2 and has the same sequence.

The clade C F4co used for immunisation was prepared using a consensus clade C sequence with the following sequence:

Amino-acid sequence of F4co clade C consensus antigen
[SEQ ID NO: 10]
MVIVQNLQGQMVHQAISPRTLNAWVKVIEEKAFSPEVIPMFTALSEGATPQDLNTMLNTVGGHQAAMQMLKDTINEEAAE
WDRLHPVHAGPIAPGQMREPRGSDIAGTTSTLQEQIAWMTSNPPIPVGDIYKRWIILGLNKIVRMYSPVSILDIKQGPKE
PFRDYVDRFFKTLRAEQATQEVKNWMTDTLLVQNANPDCKTILRALGPGATLEEMMTACQGVGGPGHKARVLHMGPISPI
ETVPVKLKPGMDGPKVKQWPLTEEKIKALTAICEEMEKEGKITKIGPENPYNTPVFAIKKKDSTKWRKLVDFRELNKRTQ
DFWEVQLGIPHPAGLKKKKSVTVLDVGDAYFSVPLDEGFRKYTAFTIPSINNETPGIRYQYNVLPQGWKGSPAIFQSSMT
KILEPFRAQNPEIVIYQYMDDLYVGSDLEIGQHRAKIEELREHLLKWGFTTPDKKHQKEPPFLKMGYELHPDKWTVQPIQ
LPEKDSWTVNDIQKLVGKLNWASQIYPGIKVRQLCKLLRGAKALTDIVPLTEEAELELAENREILKEPVHGVYYDPSKDL
IAEIQKQGHDQWTYQIYQEPFKNLKTGKYAKMRTAHTNDVKQLTEAVQKIAMESIVIWGKTPKFRLPIQKETWETWWTDY
WQATWIPEWEFVNTPPLVKLWYQLEKEPIAGAETFYVDGAANRETKIGKAGYVTDRGRQKVVSLTETTNQKTELQAIQLA
LQDSGSEVNIVTDSQYALGIIQAQPDKSESELVNQIIEQLIKKERVYLSWVPAHKGIGGNEQVDKLVSSGIRKVLAMGGK
WSKSSIVGWPAIRERMRRTEPAAEGVGAASQDLDKHGALTSSNTATNNADCAWLEAQEEEEEVGFPVRPQVPLRPMTYKA
AFDLSFFLKEKGGLEGLIYSKKRQDILDLWVYHTQGFFPDWQNYTPGPGVRYPLTFGWCYKLVPVDPREVEEANEGENNC
LLHPMSQHGMEDEDREVLKWKFDSHLARRHMARELHPEYYKDCRPMGARASILRGGKLDKWEKIRLRPGGKKHYMLKHLV
WASRELERFALNPGLLETSEGCKQIIKQLQPALQTGTEELRELYNTVATLYCVHAKIEVRDTKEALDKIEEEQNKSQQKT
QQAKAADGKVSQNYHHHHHH
Codon-optimised nucleotide sequence encoding clade C F4co:
(SEQ ID NO: 11)
ATGGTTATTGTTCAGAATCTGCAGGGTCAGATGGTTCATCAGGCAATTTCTCCGCGTACCCTGAATGCATGGGTGAAAGTGATTGAAGAA
AAAGCCTTTTCTCCGGAAGTTATTCCGATGTTTACCGCACTGAGCGAAGGTGCAACACCGCAGGATCTGAATACCATGCTGAATACCGTT
GGTGGTCATCAGGCAGCAATGCAGATGCTGAAAGATACCATTAATGAAGAGGCAGCAGAATGGGATCGTCTGCATCCGGTTCATGCAGGT
CCGATTGCACCGGGTCAGATGCGTGAACCGCGTGGTAGCGATATTGCAGGTACAACCAGCACCCTGCAAGAGCAGATTGCATGGATGACC
AGCAATCCTCCGATTCCGGTTGGTGATATTTATAAACGCTGGATTATTCTGGGCCTGAATAAAATTGTGCGTATGTATTCTCCGGTTAGC
ATTCTGGATATTAAACAGGGTCCGAAAGAACCGTTTCGTGATTATGTGGATCGCTTTTTTAAAACCCTGCGTGCAGAACAGGCAACCCAA
GAGGTTAAAAATTGGATGACCGATACCCTGCTGGTTCAGAATGCAAATCCGGATTGCAAAACCATTCTGCGTGCACTGGGTCCGGGTGCA
ACACTGGAAGAAATGATGACCGCATGTCAGGGTGTTGGTGGTCCGGGTCATAAAGCACGTGTTCTGCACATGGGTCCGATTAGCCCGATT
GAAACCGTTCCGGTGAAACTGAAACCGGGTATGGATGGTCCGAAAGTTAAACAGTGGCCTCTGACCGAAGAAAAAATCAAAGCCCTGACC
GCAATTTGTGAAGAAATGGAAAAAGAAGGCAAAATTACCAAAATTGGTCCGGAAAATCCGTATAACACACCGGTGTTTGCCATTAAAAAA
AAAGATAGCACCAAATGGCGTAAACTGGTGGATTTTCGCGAACTGAATAAACGTACCCAGGATTTTTGGGAAGTTCAGCTGGGTATTCCG
CATCCGGCAGGTCTGAAAAAAAAAAAATCCGTGACCGTTCTGGATGTTGGTGATGCCTATTTTTCTGTTCCGCTGGATGAAGGTTTTCGT
AAATATACCGCCTTTACCATTCCGAGCATTAATAATGAAACACCGGGTATTCGCTATCAGTATAATGTTCTGCCGCAGGGTTGGAAAGGT
TCTCCGGCAATTTTTCAGAGCAGCATGACCAAAATTCTGGAACCGTTTCGCGCACAGAATCCGGAAATTGTGATTTATCAGTATATGGAT
GATCTGTATGTTGGTAGCGATCTGGAAATTGGTCAGCATCGTGCCAAAATTGAAGAACTGCGTGAACATCTGCTGAAATGGGGTTTTACC
ACACCGGATAAAAAACATCAGAAAGAACCGCCGTTTCTGAAAATGGGTTATGAACTGCATCCGGATAAATGGACCGTTCAGCCGATTCAG
CTGCCGGAAAAAGATAGCTGGACCGTGAATGATATTCAGAAACTGGTGGGCAAACTGAATTGGGCAAGCCAGATTTATCCGGGTATTAAA
GTTCGTCAGCTGTGTAAACTGCTGCGTGGTGCAAAAGCACTGACCGATATTGTTCCGCTGACAGAAGAAGCAGAACTGGAACTGGCCGAA
AATCGTGAAATTCTGAAAGAACCGGTGCATGGTGTTTATTATGATCCGAGCAAAGATCTGATTGCCGAAATTCAGAAACAGGGTCATGAT
CAGTGGACCTATCAGATTTATCAGGAACCGTTTAAAAATCTGAAAACCGGCAAATATGCAAAAATGCGTACCGCACATACCAATGATGTT
AAACAGCTGACCGAAGCCGTTCAGAAAATTGCCATGGAAAGCATTGTGATTTGGGGTAAAACACCGAAATTTCGTCTGCCGATTCAGAAA
GAAACCTGGGAAACATGGTGGACCGATTATTGGCAGGCAACCTGGATTCCGGAATGGGAATTTGTTAATACACCGCCGCTGGTTAAACTG
TGGTATCAGCTGGAAAAAGAACCGATTGCAGGTGCAGAAACCTTTTATGTTGATGGTGCAGCAAATCGCGAAACCAAAATTGGCAAAGCC
GGTTATGTTACCGATCGTGGTCGTCAGAAAGTTGTTAGCCTGACCGAAACCACCAATCAGAAAACCGAACTGCAGGCAATTCAGCTGGCC
CTGCAGGATAGCGGTAGCGAAGTTAATATTGTGACCGATAGCCAGTATGCACTGGGTATTATTCAGGCACAGCCGGATAAAAGCGAAAGC
GAACTGGTGAATCAGATTATTGAACAGCTGATTAAAAAAGAACGCGTGTATCTGAGCTGGGTTCCGGCACATAAAGGTATTGGTGGCAAT
GAACAGGTTGATAAACTGGTTAGCAGCGGTATTCGTAAAGTTCTGGCCATGGGTGGTAAATGGTCTAAAAGCAGCATTGTTGGTTGGCCG
GCAATTCGTGAACGTATGCGTCGTACCGAACCGGCAGCAGAAGGTGTTGGCGCAGCAAGCCAGGATCTGGATAAACATGGTGCACTGACC
AGCAGCAATACCGCAACCAATAATGCAGATTGTGCATGGCTGGAAGCACAGGAAGAAGAAGAAGAAGTTGGTTTTCCGGTTCGTCCGCAG
GTTCCGCTGCGTCCGATGACCTATAAAGCAGCATTTGATCTGAGCTTTTTTCTGAAAGAAAAAGGTGGTCTGGAAGGTCTGATTTATAGC
AAAAAACGCCAGGATATTCTGGATCTGTGGGTTTATCATACCCAGGGTTTTTTTCCGGATTGGCAGAATTACACACCGGGTCCGGGTGTG
CGTTATCCGCTGACCTTTGGTTGGTGTTATAAACTGGTTCCGGTTGATCCGCGTGAAGTTGAAGAAGCAAACGAAGGCGAAAATAATTGT
CTGCTGCATCCGATGAGCCAGCATGGTATGGAAGATGAAGATCGCGAAGTGCTGAAATGGAAATTTGATAGCCATCTGGCTCGTCGTCAC
ATGGCACGCGAACTGCATCCGGAATATTATAAAGATTGCCGTCCGATGGGTGCACGTGCAAGCATTCTGCGTGGTGGTAAACTGGATAAA
TGGGAAAAAATTCGTCTGCGTCCGGGTGGTAAAAAACATTATATGCTGAAACATCTGGTTTGGGCAAGCCGTGAACTGGAACGTTTTGCA
CTGAATCCGGGTCTGCTGGAAACCAGCGAAGGTTGCAAACAAATTATTAAACAGCTGCAGCCGGCACTGCAGACCGGCACCGAAGAACTG
CGCAGCCTGTATAATACCGTTGCAACCCTGTATTGTGTGCATGCGAAAATTGAAGTGCGCGATACCAAAGAAGCACTGGATAAAATTGAA
GAAGAACAGAATAAAAGCCAGCAGAAAACCCAGCAGGCAAAAGCAGCAGATGGTAAAGTGAGCCAGAATTATCACCACCACCACCACCAC
TAA

For each immunization, the F4co protein was adjuvanted using AS01_{B}.

Female CB6F1 (hybrid of C57Bl/6 and Balb/C mice) of 6 to 8 weeks old were immunized three times intra-muscularly at days 0, 14 and 28 with 50 μl of the F4co clade B or clade C (3 or 9 μg) formulated in the AS01_{B} Adjuvant System.

Mice were allocated in four groups (40 animals per group):
Group 1: 9 μg F4co clade B/AS01_{B}
Group 2: 3 μg F4co clade B/AS01_{B}
Group 3: 9 μg F4co clade C/AS01_{B}
Group 4: 3 μg F4co clade C/AS01_{B}

Blood samples were taken for testing seven days after the second and third immunisations (7d pII and 7d pIII, respectively). The frequency of F4co-specific CD4+ and CD8+ T cells secreting IFN-γ and/or IL-2 and/or TNFα was determined 7 days post-second and third dose.

Briefly, peripheral blood lymphocytes (PBLs) from 40 mice/group were collected and pooled (4 pools of 10 mice/group). A red blood cells lysis was performed before plating the cells on round 96-well plates at 1 million cells per well. The cells were then restimulated in vitro with a pool of overlapping 15 mers peptides (at 1 μg/ml/peptide) covering the F4co Clade B, Clade C or Clade A sequences for 6 hours at 37° C. in presence of anti-CD28 and anti-CD49d. The sequences of clade A peptides for p24 and p17 are from the native isolate TZA173 (Tanzania) and the sequences of clade A peptides for RT and Nef come from the native isolate KE MSA4070 (Kenya). The sequence of Clade C peptides is from the ZM651 strain (for p24, p17, Nef and RT). The clade B peptides cover the sequence of the F4co clade B antigen (p24 and p17 from strain BH10, RT from strain HXB2) and Nef from strain Bru-Lai).

Cells remaining in medium (no peptide stimulation) were used as negative controls for background responses. Two hours after the co-culture with the peptide pools, brefeldin A was added to the wells (to inhibit cytokine excretion) and the cells were further incubated for 4 hours and transferred overnight at ° C. The cells were subsequently stained for the following markers: CD4, CD8, IL-2, IFN-γ and TNF-α, and analyzed by flow cytometry using a LSRII (BD Biosciences, USA) and the FlowJo software (Three Star).

### 4.2 CD4+ T Cell Responses

The cross-reactive capacity of specific CD4+ T cell responses induced by the F4co clade B or F4co clade C antigens was evaluated in mice by measuring the magnitude of HIV-specific CD4⁺ T-cell responses against clade A, clade B and C peptides. Overall, F4co-specific CD4+ T cell responses were induced by both F4co clade B and clade C antigens and were observed against all clade peptides at variable intensities. The frequency of HIV-specific CD4+ T cells was increased after the third dose with both clade B and clade C F4co antigens.

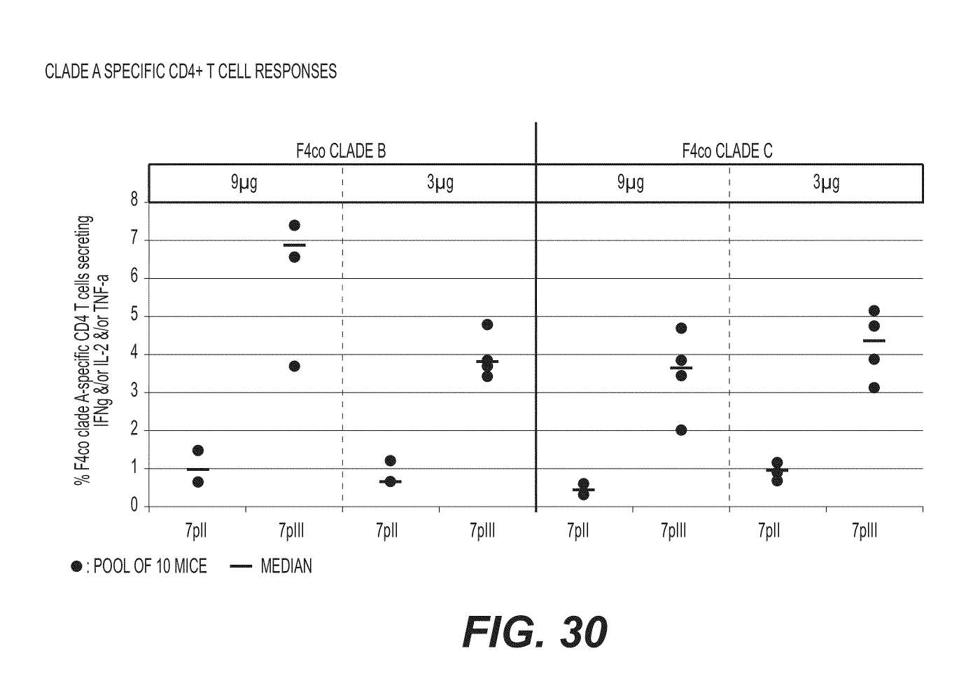

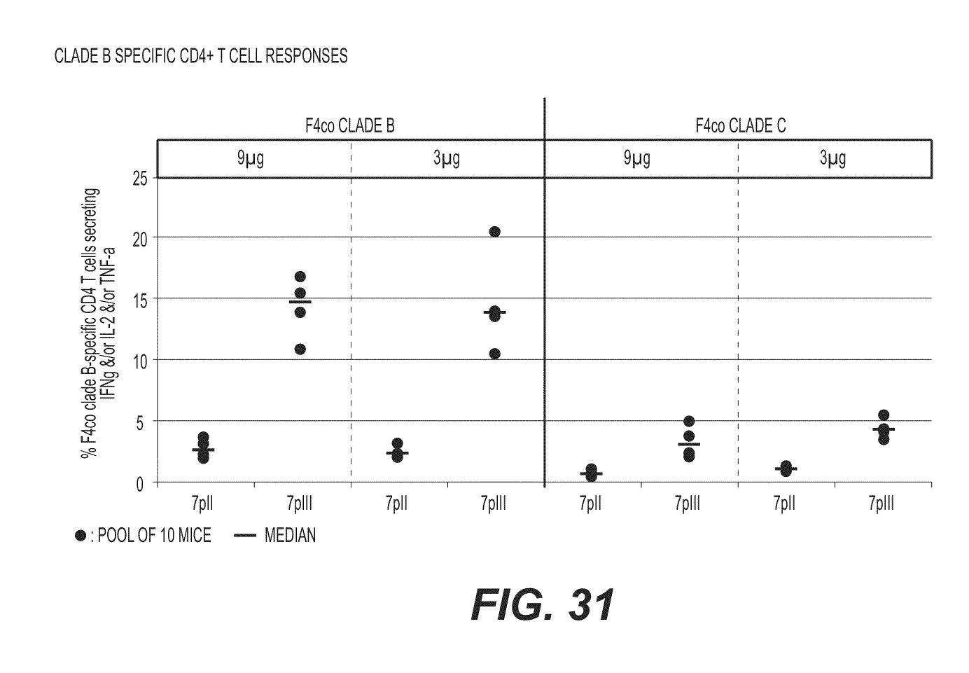

The F4co clade B antigen induced the highest level of CD4+ T cell responses against clade B peptides perhaps attributable to the fact that the peptides have exactly the same sequence as the F4co clade B antigen used for immunisation. Cross-reactivity of F4co clade B-induced CD4+ T cell responses was observed against clade A and clade C peptides. The intensity of specific CD4⁺ T-cell responses against clade A and C peptides was around half that observed with clade B peptides (FIGS. 30-32 and 36).

The magnitudes of the clade C-specific CD4+ T cell responses elicited by both F4co clade C and F4co clade B antigens were comparable (FIG. 32). Interestingly, cross-reactive HIV-specific CD4+ T cell responses were induced by the F4co clade C antigen with the intensity of CD4⁺ T-cell responses against clade A and B peptides more than half of the one obtained with clade C peptides (FIGS. 30-32 and 36).

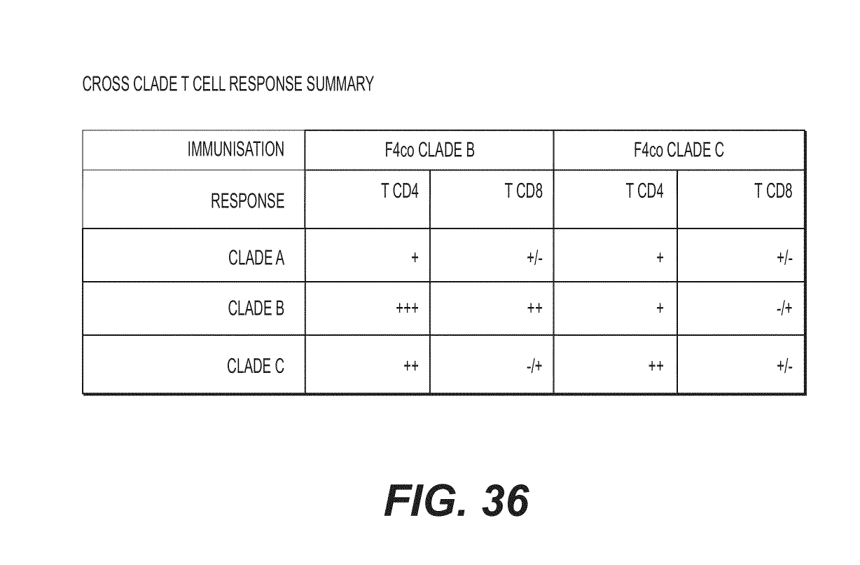

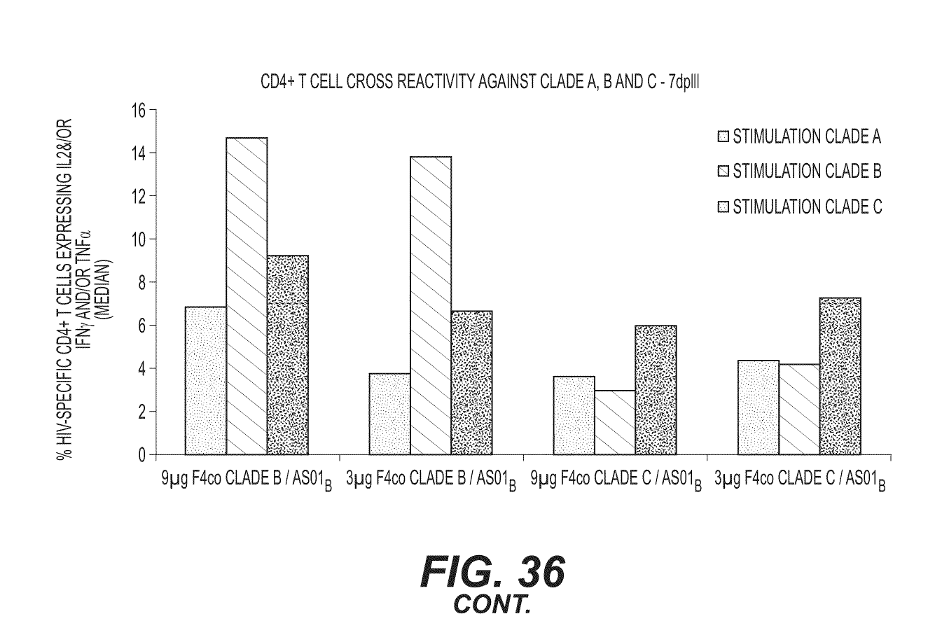

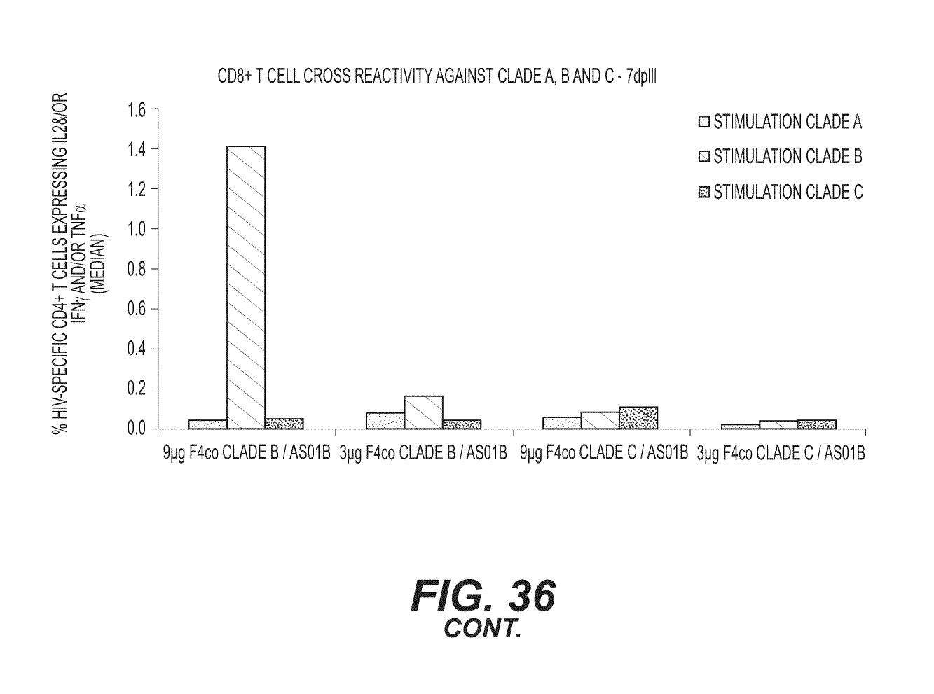

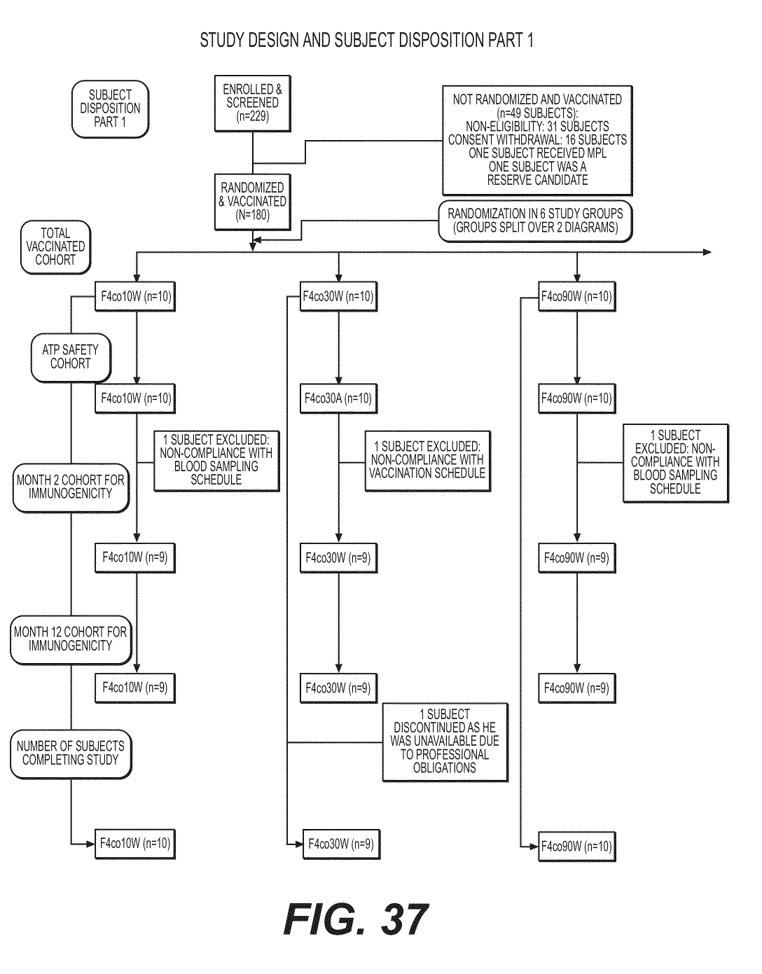

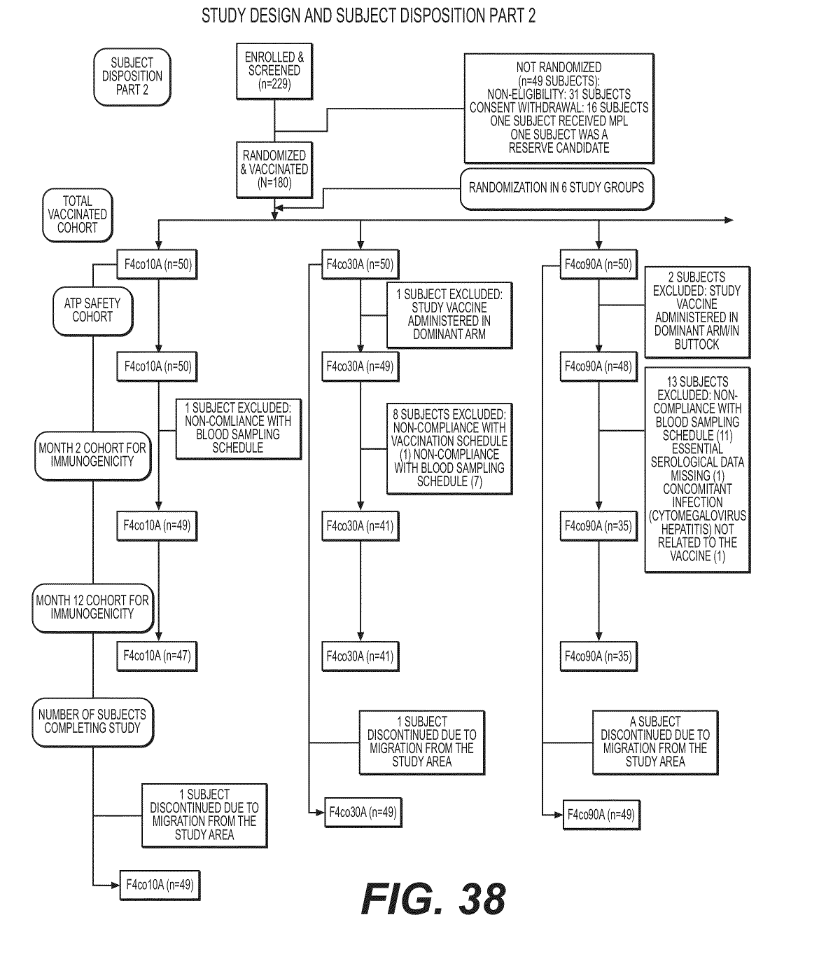

As expected, the percentage of responding CD4+ T cells were lowest against the clade A peptides, regardless the antigen used for immunisation (FIGS. 30 and 36), as these peptides have the lowest percent identity with the clade B and clade C sequences used in the F4co proteins (FIG. 8).

Additionally, F4co-specific CD4+ T cells isolated after the second and the third immunisation were found to be polyfunctional, with more than half of each population expressing IL2 as well as IFNγ and/or TNFα (data not shown).

### 4.3 CD8+ T Cell Responses

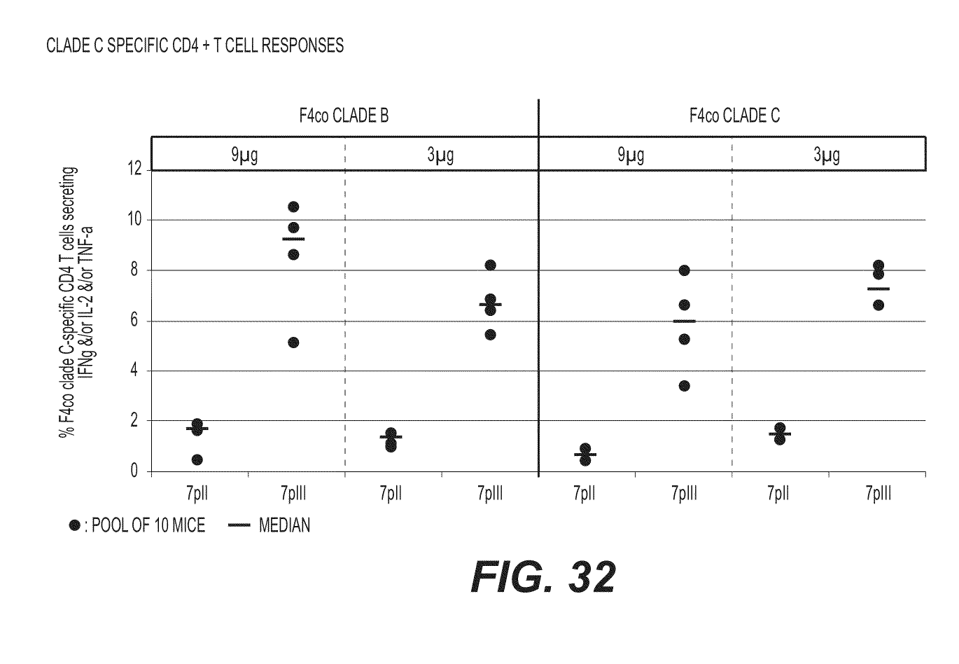

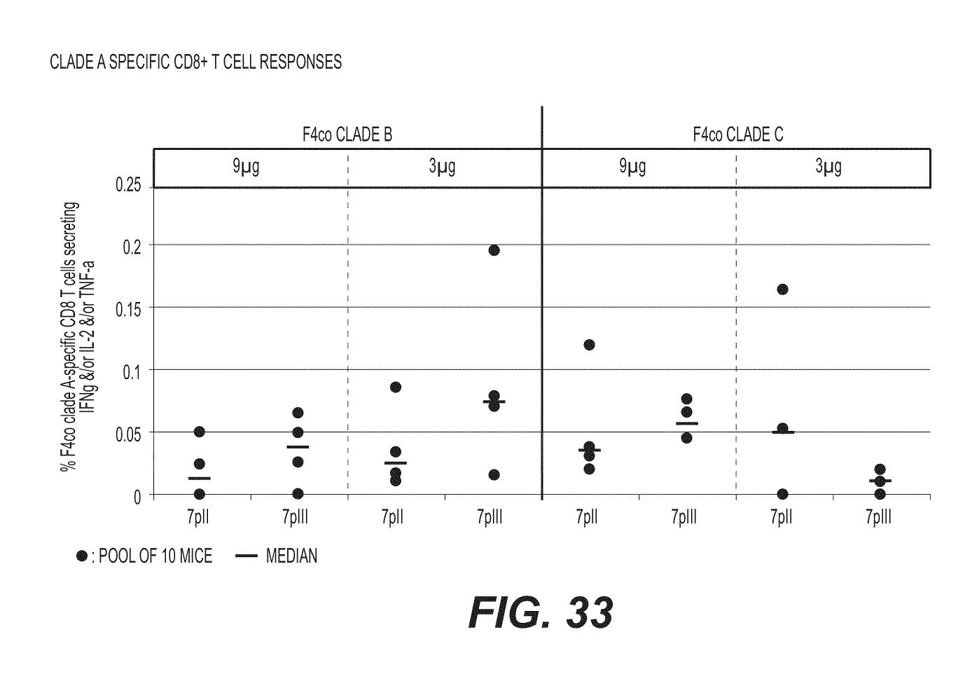

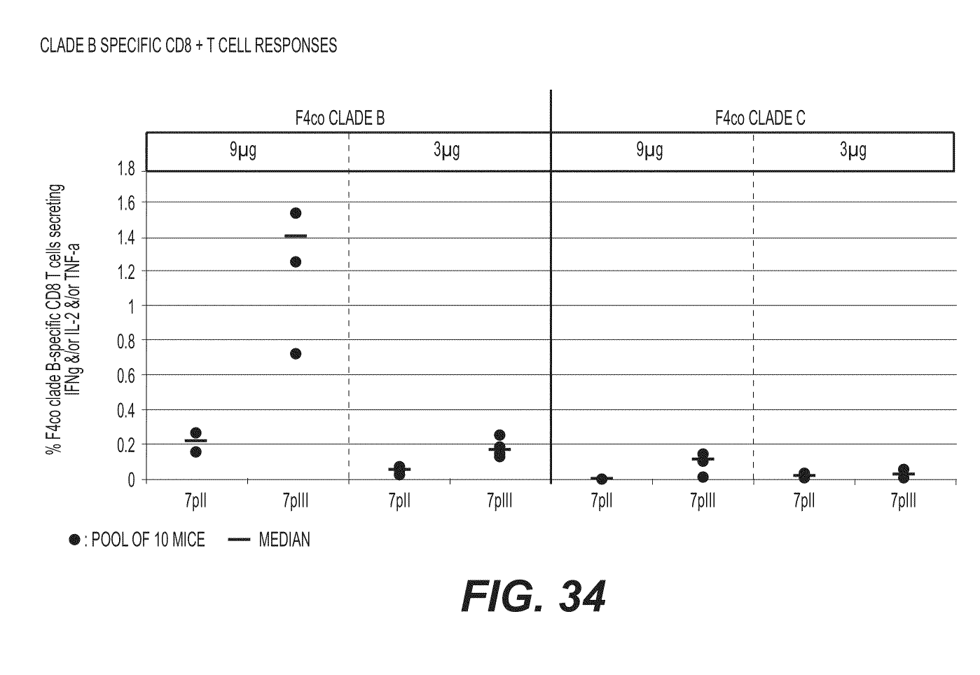

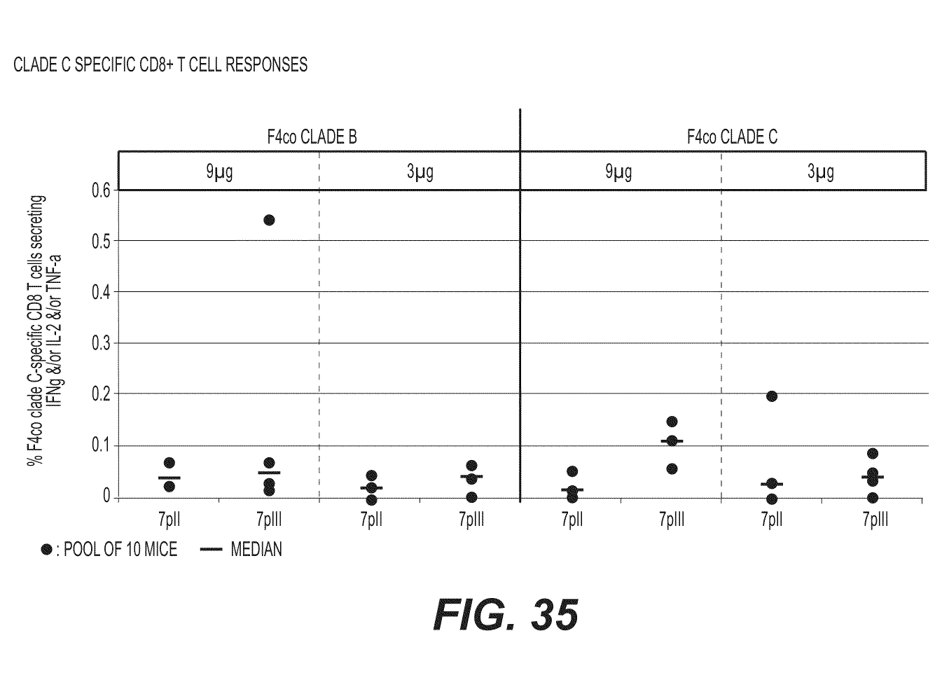

The levels of F4co-specific CD8+ T cells induced by both clade B and clade C antigens were overall lower than those observed for CD4+ T cell responses, but still detectable in some pools of animals. As with the CD4+ T cell data, the highest CD8+ T cell response was induced by the F4co clade B antigen and specific for clade B peptides. Cross-reactivity against clade C and clade A peptides, was very low (FIGS. 33-35 and 36). The F4co Clade C antigen elicited a very low CD8+ T cell response specific for clade C, but cross-reactivity against clade B and clade A peptides could be detected, albeit with low intensity.

Polyfunctionality could not be reliably analysed due to the low percentages of responder CD8+ T cells.

### 4.4 Conclusions

The cross-clade results from this preclinical study is summarised in FIG. 36. Strong cross-reactiveF4co-specifc CD4+ T cell responses against clade A, B and C were induced by both F4co clade B and F4co clade C antigens, and the responses were polyfunctional. The frequency of F4co-specific CD8+ T cells was not as strong, but a cross-clade response was detectable across all three clades tested.

1. A method of inducing an immune response against HIV-1 in a subject comprising administering to the subject an immunogenic composition comprising:
a. one or more polypeptides comprising HIV-1 antigens Nef, Pol and Gag or immunogenic fragments thereof; wherein Nef, Pol and Gag are from an HIV-1 strain of clade A, B, C, D, E, F, G, H, J, K, or a circulating recombinant form of HIV-1 (CRF); and
b. an adjuvant that is a preferential inducer of a Th1 immune response,
wherein the induced immune response is against an HIV-1 strain from one or more clades different from the one or more HIV-1 clades in the immunogenic composition.

2. The method of claim 1, wherein Nef, Pol and Gag are from an HIV-1 clade B strain.

3. The method of claim 2, wherein the polypeptides form a fusion protein comprising p24-RT-Nef-p17.

4. The method of claim 1, wherein the immunogenic composition further comprises Env or immunogenic fragment thereof.

5. The method of claim 1, wherein the adjuvant comprises an immunologically active saponin fraction and a lipopolysaccharide and optionally further comprises an immunostimulatory oligonucleotide.

6. The method of claim 5, wherein said immunologically active saponin fraction is QS21; said lipopolysaccharide is 3D-MPL; and said optional immunostimulatory oligonucleotide comprises a CpG motif.

7. The method of claim 5, wherein the adjuvant further comprises a liposome carrier or an oil-in-water emulsion.

8. The method of claim 1, wherein a humoral immune response against HIV-1 strains from said one or more clades different from the one or more HIV-1 clades in the immunogenic composition is induced in the subject.

9. The method of claim 1, wherein multiple-cytokine-producing CD4+ T cells against HIV-1 strains from said one or more clades different from the one or more HIV-1 clades in the immunogenic composition are induced in the subject, wherein the cytokines are selected from IL-2, IFNγ and/or TNFα.

10. The method of claim 1, wherein progressive CD4+ T cell decline is prevented in a subject infected with an HIV-1 strain from said one or more clades different from the one or more HIV-1 clades in the immunogenic composition.

11. The method of claim 1, wherein viral reservoirs are reduced or eliminated in a subject infected with an HIV-1 strain from said one or more clades different from the one or more HIV-1 clades in the immunogenic composition.

12. The method of claim 1, wherein HIV-1-specific polyfunctional CD4+ T-cells are induced in a subject infected with an HIV-1 strain from said one or more clades different from the one or more HIV-1 clades in the immunogenic composition.

13. The method of claim 1, wherein viremia is reduced or controlled in an subject infected with an HIV-1 strain from said one or more clades different from the one or more HIV-1 clades in the immunogenic composition.

14. The method of claim 1, wherein a long term immune response against HIV-1 strains from said one or more clades different from the one or more HIV-1 clades in the immunogenic composition is induced in the subject.

15. The method of claim 1, wherein the immunogenic composition is administered to the subject as two or three doses, wherein the doses are separated by a period of two weeks to three months.

16. The method of claim 15, wherein the immunogenic composition is administered to a subject every 6-24 months.

17. The method of claim 1, wherein the immunogenic composition wherein the composition is used as part of a prime-boost regimen.

18. A method of treating or preventing HIV-1 infection in a subject comprising administering to the subject an immunogenic composition comprising:
a. one or more polypeptides comprising HIV-1 antigens Nef, Pol and Gag or immunogenic fragments thereof; wherein Nef, Pol and Gag are from an HIV-1 strain of clade A, B, C, D, E, F, G, H, J, K, or a circulating recombinant form of HIV-1 (CRF); and
b. an adjuvant that is a preferential inducer of a Th1 immune response, wherein the HIV-1 infection being treated or prevented is from one or more HIV-1 clades different from the one or more HIV-1 clades in the immunogenic composition.

19. The method of claim 18, wherein the subject is infected with HIV-1 prior to administration of the immunogenic composition.
